(19) United States
(12) Patent Application Publication
(10) Pub. No.: US 2026/0308702 A1
(43) Pub. Date: Oct. 8, 2026

(54) COMPOSITIONS AND METHODS FOR THE DETECTION OF A CHROMOSOMAL TRANSLOCATION IN BRASSICA NAPUS

(71) Applicant: PIONEER HI-BRED INTERNATIONAL, INC., JOHNSTON, IA (US)

(72) Inventors: DAVID GEORGE CHARNE, GUELPH (CA); KEVIN A. FENGLER, CLIVE, IA (US); SIVA S. AMMIRAJU JETTY, JOHNSTON, IA (US); SCOTT CHARLES JOBGEN, WEST DES MOINES, IA (US)

(73) Assignee: PIONEER HI-BRED INTERNATIONAL, INC., JOHNSTON, IA (US)

(21) Appl. No.: 19/710,710

(22) Filed: Jun. 17, 2026

Related U.S. Application Data
(63) Continuation of application No. 17/762,805, filed on Sep. 24, 2020, now Pat. No. 12,698,539.
(60) Provisional application No. 62/906,352, filed on Sep. 26, 2019.

(51) Int. Cl. C12Q 1/6895

(52) U.S. Cl. CPC C12Q 1/6895; C12Q 2600/13; C12Q 2600/156

(57) ABSTRACT

Provided are compositions and methods for the detection of a chromosomal location in Brassica napus. This disclosure is generally related to the field of plant molecular biology, and in particular embodiments, to the field of detecting a chromosomal translocation within plants. In certain embodiments, the chromosomal translocation occurs between the N7 and N16 chromosomes of Brassica napus. In other embodiments, the chromosomal translocation is a homoeologous chromosomal reciprocal translocation. Accordingly, this disclosure provides compositions and methods for the identification, detection and utilization of such a chromosomal translocation.

### CROSS-REFERENCE TO RELATED APPLICATION

This application claims the benefit of PCT/US20/52443, filed Sep. 24, 2020 which claims the benefit of U.S. Provisional Patent Application Ser. No. 62/906,352, filed on Sep. 26, 2019, the disclosure of which is hereby incorporated in its entirety by this reference.

### REFERENCE TO A SEQUENCE LISTING

The official copy of the sequence listing is submitted electronically via EFS-Web as an ASCII formatted sequence listing with a file named “Bnapus Chromosomal Translocation 2_ST25”, created on Sep. 22, 2020, and having a size of 191 kilobytes and is filed concurrently with the specification. The sequence listing contained in this ASCII formatted document is part of the specification and is herein incorporated by reference in its entirety.

### FIELD OF THE INVENTION

This disclosure is generally related to the field of plant molecular biology, and in particular embodiments, to the field of detecting a chromosomal translocation within plants. In certain aspects, the chromosomal translocation occurs between the N7 and N16 chromosomes of Brassica napus. In other aspects, the chromosomal translocation is a homoeologous chromosomal reciprocal translocation. Accordingly, this disclosure provides compositions and methods for the identification, detection and utilization of such a chromosomal translocation.

### BACKGROUND

Brassica napus (canola) is one of the major economic crops grown worldwide as a primary source of vegetable oil. Growing demand for diets with healthy oil has increased B. napus importance as a food. B. napus is one of the lowest-cost sources of vegetable oil. B. napus oil accounts for a large proportion of edible oil consumed worldwide. It is used as a liquid oil, in baking, frying, salad dressing, margarine, and a multitude of processed foods. B. napus is agronomically well-adapted to many parts of the world and production continues to expand from year to year. The low cost and ready availability of B. napus oil provides an excellent opportunity to upgrade this commodity oil into higher value specialty oils that add value to the farmer. The development of new and improved lines of B. napus remains a continual challenge for breeders.

B. napus is a recently formed allo-tetraploid species that contains two closely related homoeologous sub-genome types, named A and C. Although a tetraploid, canola behaves genetically as a diploid, due to strict control on the meiotic chromosome pairing that prevent recombination between highly similar homoeologous chromosomes. However, “de novo” recombination can occur between A and C genomes at low frequency in ‘natural’ polyploids of canola (unlike re-synthesized canola, where these events occur at a higher frequency), leading to a variety of large homoeologous chromosomal structural rearrangements (HCR). Few historical HCR are artificially selected and fixed in the canola germplasm inadvertently due to their linkage with traits of commercial importance. One class of HCR is reciprocal translocation (homoeologous chromosomal reciprocal translocation), where homoeologous recombination result in reciprocal swapping of parts of the A and C chromosomes without loss of any genetic material. Progenies derived from crosses between B. napus plants differing for the presence or absence of an homoeologous chromosomal reciprocal translocation can be classified into two groups. Those with ‘parental type’ chromosomal constitution with no apparent loss or gain of genetic material, and those with ‘non-parental recombinant type’ chromosomal constitution that result in duplication or deletion of genetic material. The second, will result in complex non-mendelian segregation patterns. Few ‘fixed’ homoeologous chromosomal reciprocal translocations have been identified and reported in the literature for B. napus.

Molecular breeding assays that can be utilized for breeding new lines of B. napus are desirable. For example, assays that can be used to detect and monitor homoeologous chromosomal reciprocal translocations within B. napus are of particular importance.

Therefore, there is an important need in the art to continue to identify novel methods and compositions for molecular breeding assays that can be readily applied for breeding in B. napus.

### SUMMARY

Disclosed herein are sequences, constructs, and methods for the detection of an a N7/N16 homoeologous chromosomal reciprocal translocation in a canola plant, tissue, or cell. In some aspects the disclosure relates to the detection of at least one PCR amplicon of the N7/N16 homoeologous chromosomal reciprocal translocation, wherein the one or more PCR amplicons localize within either SEQ ID NO:2 or SEQ ID NO:4. In additional aspect, the disclosure relates to screening the first canola plant, tissue, or cell for the N7/N16 homoeologous chromosomal reciprocal translocation. In other aspects, the disclosure relates to selecting the first canola plant, tissue, or cell or selecting a progeny of the first canola plant, tissue, or cell whereby the resulting plant, tissue, or cell displays the N7/N16 homoeologous chromosomal reciprocal translocation. In further aspects, the amplicon of SEQ ID NO:2 comprises a primer associated with any one of SEQ ID NO:5493-8026, SEQ ID NO:9, SEQ ID NO:10, SEQ ID NO:14, SEQ ID NO:16 or a complement thereof. Accordingly, the amplicon of SEQ ID NO:2 comprises a forward or 5′ primer associated with any one of SEQ ID NO:5493-8026, SEQ ID NO:9, SEQ ID NO:10, SEQ ID NO:14, SEQ ID NO:16 or a complement thereof. Likewise, the amplicon of SEQ ID NO:2 comprises a reverse or 3′ primer associated with any one of SEQ ID NO:5493-8026, SEQ ID NO:9, SEQ ID NO:10, SEQ ID NO:14, SEQ ID NO:16 or a complement thereof. In some aspects, the amplicon of SEQ ID NO:2 comprises a probe associated with any one of SEQ ID NO:372-488 or a complement thereof. In additional aspects, the amplicon of SEQ ID NO:4 comprises a primer associated with any one of SEQ ID NO: 8027-10560, SEQ ID NO:11, SEQ ID NO:12, SEQ ID NO:18, SEQ ID NO:20 or a complement thereof. Accordingly, the amplicon of SEQ ID NO:4 comprises a forward or 5′ primer associated with any one of SEQ ID NO: 8027-10560, SEQ ID NO: 11, SEQ ID NO:12, SEQ ID NO:18, SEQ ID NO:20 or a complement thereof. Likewise, the amplicon of SEQ ID NO:4 comprises a reverse or 3′ primer associated with any one of SEQ ID NO: 8027-10560, SEQ ID NO:11, SEQ ID NO:12, SEQ ID NO:18, SEQ ID NO:20 or a complement thereof. In other aspects, the amplicon of SEQ ID NO:4 comprises a probe associated with any one of SEQ ID NO:255-371 or a complement thereof. In further aspects, the amplicon of SEQ ID NO:2 comprises a polynucleotide that shares at least 98% sequence identity with a primer associated with any one of SEQ ID NO:5493-8026, SEQ ID NO:9, SEQ ID NO:10, SEQ ID NO:14, SEQ ID NO:16 or a complement thereof. In other aspects, the amplicon of SEQ ID NO:2 comprises a polynucleotide that shares at least 95%, 96%, 97%, 98%, or 99% sequence identity with a primer associated with any one of SEQ ID NO:5493-8026, SEQ ID NO:9, SEQ ID NO:10, SEQ ID NO:14, SEQ ID NO:16 or a complement thereof. In an aspect, the amplicon of SEQ ID NO:2 comprises a probe that shares at least 95%, 96%, 97%, 98%, or 99% sequence identity with a probe associated with any one of SEQ ID NO:372-488 or a complement thereof. In some aspects, the amplicon of SEQ ID NO:4 comprises a polynucleotide that shares at least 98% sequence identity with a primer associated with any one of SEQ ID NO: 8027-10560, SEQ ID NO:11, SEQ ID NO:12, SEQ ID NO:18, SEQ ID NO:20 or a complement thereof. In further aspects, the amplicon of SEQ ID NO:4 comprises a polynucleotide that shares at least 95%, 96%, 97%, 98%, or 99% sequence identity with a primer associated with any one SEQ ID NO: 8027-10560, SEQ ID NO:11, SEQ ID NO:12, SEQ ID NO:18, SEQ ID NO:20 or a complement thereof. In a further aspect, the amplicon of SEQ ID NO:4 comprises a probe that shares at least 95%, 96%, 97%, 98%, or 99% sequence identity with a probe associated with any one of SEQ ID NO:255-371 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

Disclosed herein are sequences, constructs, and methods for the detection of an a N7/N16 homoeologous chromosomal reciprocal translocation in a canola plant, tissue, or cell. In some aspects the disclosure relates to the detection of at least one PCR amplicon associated with the N7/N16 homoeologous chromosomal reciprocal translocation, wherein the one or more PCR amplicons localize within either SEQ ID NO:1 or SEQ ID NO:3. In additional aspect, the disclosure relates to screening the first canola plant, tissue, or cell for the N7/N16 homoeologous chromosomal reciprocal translocation. In other aspects, the disclosure relates to selecting the first canola plant, tissue, or cell or selecting a progeny of the first canola plant, tissue, or cell whereby the resulting plant, tissue, or cell displays the N7/N16 homoeologous chromosomal reciprocal translocation. In further aspects, the amplicon of SEQ ID NO:1 comprises a primer associated with any one of SEQ ID NO: 489-2990, SEQ ID NO:5 SEQ ID NO:6, SEQ ID NO:13, SEQ ID NO:15 or a complement thereof. Accordingly, the amplicon of SEQ ID NO:1 comprises a forward or 5′ primer associated with any one of SEQ ID NO: 489-2990, SEQ ID NO:5 SEQ ID NO:6, SEQ ID NO:13, SEQ ID NO:15 or a complement thereof. Likewise, the amplicon of SEQ ID NO:1 comprises a reverse or 3′ primer associated with any one of SEQ ID NO: 489-2990, SEQ ID NO:5 SEQ ID NO:6, SEQ ID NO:13, SEQ ID NO:15 or a complement thereof. In some aspects, the amplicon of SEQ ID NO:1 comprises a probe associated with any one of SEQ ID NO:138-254 or a complement thereof. In additional aspects, the amplicon of SEQ ID NO:3 comprises a primer associated with any one of SEQ ID NO: 2991-5492, SEQ ID NO:7, SEQ ID NO:8, SEQ ID NO: 17, SEQ ID NO:19 or a complement thereof. Accordingly, the amplicon of SEQ ID NO:3 comprises a forward or 5′ primer associated with any one of SEQ ID NO: 2991-5492, SEQ ID NO:7, SEQ ID NO:8, SEQ ID NO:17, SEQ ID NO:19 or a complement thereof. Likewise, the amplicon of SEQ ID NO:3 comprises a reverse or 3′ primer associated with any one of SEQ ID NO: 2991-5492, SEQ ID NO:7, SEQ ID NO:8, SEQ ID NO:17, SEQ ID NO:19 or a complement thereof. In other aspects, the amplicon of SEQ ID NO:3 comprises a probe associated with any one of SEQ ID NO: 21-137 or a complement thereof. In further aspects, the amplicon of SEQ ID NO:1 comprises a polynucleotide that shares at least 98% sequence identity with a primer associated with any one of SEQ ID NO: 489-2990, SEQ ID NO:5 SEQ ID NO:6, SEQ ID NO:13, SEQ ID NO:15 or a complement thereof. In other aspects, the amplicon of SEQ ID NO:1 comprises a polynucleotide that shares at least 95%, 96%, 97%, 98%, or 99% sequence identity with a primer associated with any one of SEQ ID NO: 489-2990, SEQ ID NO:5 SEQ ID NO:6, SEQ ID NO:13, SEQ ID NO:15 or a complement thereof. In an aspect, the amplicon of SEQ ID NO:1 comprises a probe that shares at least 95%, 96%, 97%, 98%, or 99% sequence identity with a probe associated with any one of SEQ ID NO:138-254 or a complement thereof. In some aspects, the amplicon of SEQ ID NO:3 comprises a polynucleotide that shares at least 98% sequence identity with a primer associated with any one of SEQ ID NO: 2991-5492, SEQ ID NO:7, SEQ ID NO:8, SEQ ID NO:17, SEQ ID NO:19 or a complement thereof. In further aspects, the amplicon of SEQ ID NO:3 comprises a polynucleotide that shares at least 95%, 96%, 97%, 98%, or 99% sequence identity with a primer associated with any one SEQ ID NO: 2991-5492, SEQ ID NO:7, SEQ ID NO:8, SEQ ID NO:17, SEQ ID NO:19 or a complement thereof. In a further aspect, the amplicon of SEQ ID NO:3 comprises a probe that shares at least 95%, 96%, 97%, 98%, or 99% sequence identity with a probe associated with any one of SEQ ID NO: 21-137 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In other aspects, the disclosure relates to a canola plant selected using the methods and compositions of the subject disclosure. In an aspect, the canola plant can be a progeny canola plant obtained from the canola plant selected using the methods and compositions of the subject disclosure. In further aspects, the canola plant comprises a canola seed. Exemplary structures of the canola seed include the outer hull and the embryo. In other aspects, the canola plant comprises a canola plant part. Exemplary canola plant parts include plant cells, plant protoplasts, plant cell tissue cultures from which plants can be regenerated, plant DNA, plant calli, plant clumps, and plant cells that are intact in plants or parts of plants, such as embryos, pollen, ovules, flowers, seeds, leaves, roots, root tips, anthers, and the like. In such embodiments, the canola plant is assayed for the presence or absence of the N7/N16 homoeologous chromosomal reciprocal translocation using the compositions and methods of the subject disclosure.

In an aspect, the disclosure relates to introgressing the N7/N16 homoeologous chromosomal reciprocal translocation into a progeny canola plant. In a further aspect, the disclosure relates to crossing a parent canola plant with another parent canola plant, wherein at least one parent canola plant comprises the N7/N16 homoeologous chromosomal reciprocal translocation. In another aspect, the disclosure relates to harvesting a progeny seed from the crossing as described previously, wherein the progeny seed comprises the N7/N16 homoeologous chromosomal reciprocal translocation. In an additional aspect, the disclosure relates to planting the progeny seed. In a further aspect, the disclosure relates to growing the progeny seed, wherein the progeny seed produce a progeny canola plant, wherein the progeny canola plant comprises the N7/N16 homoeologous chromosomal reciprocal translocation.

In some aspects the disclosure relates to introgressing the N7/N16 homoeologous chromosomal reciprocal translocation into a progeny canola plant, wherein introgression occurs within either SEQ ID NO:2 or SEQ ID NO:4. In further aspects, the introgression of the N7/N16 homoeologous chromosomal reciprocal translocation into a progeny canola plant is detected by producing an amplicon of SEQ ID NO:2, wherein the amplicon comprises a primer associated with any one of SEQ ID NO:5493-8026, SEQ ID NO:9, SEQ ID NO:10, SEQ ID NO:14, SEQ ID NO:16 or a complement thereof. Accordingly, the amplicon of SEQ ID NO:2 comprises a forward or 5′ primer associated with any one of SEQ ID NO:5493-8026, SEQ ID NO:9, SEQ ID NO:10, SEQ ID NO:14, SEQ ID NO:16 or a complement thereof. Likewise, the amplicon of SEQ ID NO:2 comprises a reverse or 3′ primer associated with any one of SEQ ID NO:5493-8026, SEQ ID NO:9, SEQ ID NO:10, SEQ ID NO:14, SEQ ID NO:16 or a complement thereof. In some aspects, the amplicon of SEQ ID NO:2 comprises a probe associated with any one of SEQ ID NO:372-488 or a complement thereof. In other aspects, the introgression of the N7/N16 homoeologous chromosomal reciprocal translocation into a progeny canola plant is detected by producing an amplicon of SEQ ID NO:4, wherein the amplicon comprises a primer associated with any one of SEQ ID NO: 8027-10560, SEQ ID NO:11, SEQ ID NO:12, SEQ ID NO:18, SEQ ID NO:20 or a complement thereof. Accordingly, the amplicon of SEQ ID NO:4 comprises a forward or 5′ primer associated with any one of SEQ ID NO: 8027-10560, SEQ ID NO:11, SEQ ID NO:12, SEQ ID NO:18, SEQ ID NO:20 or a complement thereof. Likewise, the amplicon of SEQ ID NO:4 comprises a reverse or 3′ primer associated with any one of SEQ ID NO: 8027-10560, SEQ ID NO: 11, SEQ ID NO:12, SEQ ID NO:18, SEQ ID NO:20 or a complement thereof. In other aspects, the amplicon of SEQ ID NO:4 comprises a probe associated with any one of SEQ ID NO:255-371 or a complement thereof. In further aspects, the amplicon of SEQ ID NO:2 comprises a polynucleotide that shares at least 98% sequence identity with a primer associated with any one of SEQ ID NO:5493-8026, SEQ ID NO:9, SEQ ID NO:10, SEQ ID NO:14, SEQ ID NO:16 or a complement thereof. In other aspects, the amplicon of SEQ ID NO:2 comprises a polynucleotide that shares at least 95%, 96%, 97%, 98%, or 99% sequence identity with a primer associated with any one of SEQ ID NO:5493-8026, SEQ ID NO:9, SEQ ID NO:10, SEQ ID NO:14, SEQ ID NO:16 or a complement thereof. In an aspect, the amplicon of SEQ ID NO:2 comprises a probe that shares at least 95%, 96%, 97%, 98%, or 99% sequence identity with a probe associated with any one of SEQ ID NO:372-488 or a complement thereof. In some aspects, the amplicon of SEQ ID NO:4 comprises a polynucleotide that shares at least 98% sequence identity with a primer associated with any one of SEQ ID NO: 8027-10560, SEQ ID NO: 11, SEQ ID NO:12, SEQ ID NO:18, SEQ ID NO:20 or a complement thereof. In further aspects, the amplicon of SEQ ID NO:4 comprises a polynucleotide that shares at least 95%, 96%, 97%, 98%, or 99% sequence identity with a primer associated with any one SEQ ID NO: 8027-10560, SEQ ID NO: 11, SEQ ID NO:12, SEQ ID NO:18, SEQ ID NO:20 or a complement thereof. In a further aspect, the amplicon of SEQ ID NO:4 comprises a probe that shares at least 95%, 96%, 97%, 98%, or 99% sequence identity with a probe associated with any one of SEQ ID NO:255-371 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In some aspects the disclosure relates to introgressing the N7/N16 homoeologous chromosomal reciprocal translocation into a progeny canola plant, wherein introgression occurs within either SEQ ID NO:1 or SEQ ID NO:3. In further aspects, the introgression of the N7/N16 homoeologous chromosomal reciprocal translocation into a progeny canola plant is detected by producing an amplicon of SEQ ID NO:1, wherein the amplicon comprises a primer associated with any one of SEQ ID NO: 489-2990, SEQ ID NO:5 SEQ ID NO:6, SEQ ID NO:13, SEQ ID NO:15 or a complement thereof. Accordingly, the amplicon of SEQ ID NO:1 comprises a forward or 5′ primer associated with any one of SEQ ID NO: 489-2990, SEQ ID NO:5 SEQ ID NO:6, SEQ ID NO:13, SEQ ID NO:15 or a complement thereof. Likewise, the amplicon of SEQ ID NO:1 comprises a reverse or 3′ primer associated with any one of SEQ ID NO: 489-2990, SEQ ID NO:5 SEQ ID NO:6, SEQ ID NO:13, SEQ ID NO:15 or a complement thereof. In some aspects, the amplicon of SEQ ID NO:1 comprises a probe associated with any one of SEQ ID NO:138-254 or a complement thereof. In further aspects, the introgression of the N7/N16 homoeologous chromosomal reciprocal translocation into a progeny canola plant is detected by producing an amplicon of SEQ ID NO:3, wherein the amplicon comprises a primer associated with any one of SEQ ID NO: 2991-5492, SEQ ID NO:7, SEQ ID NO:8, SEQ ID NO:17, SEQ ID NO:19 or a complement thereof. Accordingly, the amplicon of SEQ ID NO:3 comprises a forward or 5′ primer associated with any one of SEQ ID NO: 2991-5492, SEQ ID NO:7, SEQ ID NO:8, SEQ ID NO:17, SEQ ID NO:19 or a complement thereof. Likewise, the amplicon of SEQ ID NO:3 comprises a reverse or 3′ primer associated with any one of SEQ ID NO: 2991-5492, SEQ ID NO:7, SEQ ID NO:8, SEQ ID NO:17, SEQ ID NO:19 or a complement thereof. In other aspects, the amplicon of SEQ ID NO:3 comprises a probe associated with any one of SEQ ID NO: 21-137 or a complement thereof. In further aspects, the amplicon of SEQ ID NO:1 comprises a polynucleotide that shares at least 98% sequence identity with a primer associated with any one of SEQ ID NO: 489-2990, SEQ ID NO:5 SEQ ID NO:6, SEQ ID NO:13, SEQ ID NO:15 or a complement thereof. In other aspects, the amplicon of SEQ ID NO:1 comprises a polynucleotide that shares at least 95%, 96%, 97%, 98%, or 99% sequence identity with a primer associated with any one of SEQ ID NO: 489-2990, SEQ ID NO:5 SEQ ID NO:6, SEQ ID NO:13, SEQ ID NO:15 or a complement thereof. In an aspect, the amplicon of SEQ ID NO:1 comprises a probe that shares at least 95%, 96%, 97%, 98%, or 99% sequence identity with a probe associated with any one of SEQ ID NO:138-254 or a complement thereof. In some aspects, the amplicon of SEQ ID NO:3 comprises a polynucleotide that shares at least 98% sequence identity with a primer associated with any one of SEQ ID NO: 2991-5492, SEQ ID NO:7, SEQ ID NO:8, SEQ ID NO:17, SEQ ID NO:19 or a complement thereof. In further aspects, the amplicon of SEQ ID NO:3 comprises a polynucleotide that shares at least 95%, 96%, 97%, 98%, or 99% sequence identity with a primer associated with any one SEQ ID NO: 2991-5492, SEQ ID NO:7, SEQ ID NO:8, SEQ ID NO:17, SEQ ID NO:19 or a complement thereof. In a further aspect, the amplicon of SEQ ID NO:3 comprises a probe that shares at least 95%, 96%, 97%, 98%, or 99% sequence identity with a probe associated with any one of SEQ ID NO: 21-137 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In another aspect, the disclosure relates to a PCR assay method for determining zygosity of a N7/N16 homoeologous chromosomal reciprocal translocation in a canola plant, tissue, or cell. In some aspects, the disclosure relates to performing a first PCR assay using a first probe, a first forward primer, and a first reverse primer on a polynucleotide from a canola plant sample. In other aspects, the disclosure relates to performing a second PCR assay using a second probe, a second forward primer, and a second reverse primer on the polynucleotide sample. In further aspects, the disclosure relates to quantifying the first probe and the second probe. In additional aspect, the disclosure relates to comparing the quantified first probe and the quantified second probe of the first PCR assay and the second PCR assay to determine the zygosity, wherein the zygosity of the N7/N16 homoeologous chromosomal reciprocal translocation is selected from the group consisting of homozygous, heterozygous, hemizygous, and nullizygous. In an aspect, the first and the second PCR assays are a multiplex PCR-format. In another aspect, the first and second PCR assays are performed in a single PCR assay tube or well. In an additional aspect, the first and the second PCR assays are a real-time PCR. In other aspects, the first probe and the second probe are quantified by measuring excitation/emission spectra emitted from the fluorescent dyes, during the amplification. In further aspects, the zygosity is determined by comparing the quantified first probe and the second probe using a ΔΔCt formula.

In some aspects the disclosure relates to a PCR assay method for determining zygosity of a N7/N16 homoeologous chromosomal reciprocal translocation in a canola plant, tissue, or cell, wherein PCR assay occurs within either SEQ ID NO:2 or SEQ ID NO:4. In further aspects, the PCR assay to determine zygosity of the N7/N16 homoeologous chromosomal reciprocal translocation into a progeny canola plant is detected by producing an amplicon of SEQ ID NO:2, wherein the amplicon comprises a primer associated with any one of SEQ ID NO:5493-8026, SEQ ID NO:9, SEQ ID NO:10, SEQ ID NO:14, SEQ ID NO:16 or a complement thereof. Accordingly, the amplicon of SEQ ID NO:2 comprises a forward or 5′ primer associated with any one of SEQ ID NO:5493-8026, SEQ ID NO:9, SEQ ID NO:10, SEQ ID NO:14, SEQ ID NO:16 or a complement thereof. Likewise, the amplicon of SEQ ID NO:2 comprises a reverse or 3′ primer associated with any one of SEQ ID NO:5493-8026, SEQ ID NO:9, SEQ ID NO:10, SEQ ID NO:14, SEQ ID NO:16 or a complement thereof. In some aspects, the amplicon of SEQ ID NO:2 comprises a probe associated with any one of SEQ ID NO:372-488 or a complement thereof. In other aspects, the PCR assay method for determining zygosity of a N7/N16 homoeologous chromosomal reciprocal translocation in a canola plant is detected by producing an amplicon of SEQ ID NO:4, wherein the amplicon comprises a primer associated with any one of SEQ ID NO: 8027-10560, SEQ ID NO: 11, SEQ ID NO:12, SEQ ID NO:18, SEQ ID NO:20 or a complement thereof. Accordingly, the amplicon of SEQ ID NO:4 comprises a forward or 5′ primer associated with any one of SEQ ID NO: 8027-10560, SEQ ID NO:11, SEQ ID NO:12, SEQ ID NO:18, SEQ ID NO:20 or a complement thereof. Likewise, the amplicon of SEQ ID NO:4 comprises a reverse or 3′ primer associated with any one of SEQ ID NO: 8027-10560, SEQ ID NO:11, SEQ ID NO:12, SEQ ID NO:18, SEQ ID NO:20 or a complement thereof. In other aspects, the amplicon of SEQ ID NO:4 comprises a probe associated with any one of SEQ ID NO:255-371 or a complement thereof. In further aspects, the amplicon of SEQ ID NO:2 comprises a polynucleotide that shares at least 98% sequence identity with a primer associated with any one of SEQ ID NO:5493-8026, SEQ ID NO:9, SEQ ID NO:10, SEQ ID NO:14, SEQ ID NO:16 or a complement thereof. In other aspects, the amplicon of SEQ ID NO:2 comprises a polynucleotide that shares at least 95%, 96%, 97%, 98%, or 99% sequence identity with a primer associated with any one of SEQ ID NO:5493-8026, SEQ ID NO:9, SEQ ID NO:10, SEQ ID NO:14, SEQ ID NO:16 or a complement thereof. In an aspect, the amplicon of SEQ ID NO:2 comprises a probe that shares at least 95%, 96%, 97%, 98%, or 99% sequence identity with a probe associated with any one of SEQ ID NO:372-488 or a complement thereof. In some aspects, the amplicon of SEQ ID NO:4 comprises a polynucleotide that shares at least 98% sequence identity with a primer associated with any one of SEQ ID NO: 8027-10560, SEQ ID NO:11, SEQ ID NO:12, SEQ ID NO:18, SEQ ID NO:20 or a complement thereof. In further aspects, the amplicon of SEQ ID NO:4 comprises a polynucleotide that shares at least 95%, 96%, 97%, 98%, or 99% sequence identity with a primer associated with any one SEQ ID NO: 8027-10560, SEQ ID NO: 11, SEQ ID NO:12, SEQ ID NO:18, SEQ ID NO:20 or a complement thereof. In a further aspect, the amplicon of SEQ ID NO:4 comprises a probe that shares at least 95%, 96%, 97%, 98%, or 99% sequence identity with a probe associated with any one of SEQ ID NO:255-371 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In some aspects the disclosure relates to a PCR assay method for determining zygosity of a N7/N16 homoeologous chromosomal reciprocal translocation in a canola plant, tissue, or cell, wherein PCR assay occurs within either SEQ ID NO:1 or SEQ ID NO:3. In further aspects, the PCR assay to determine zygosity of the N7/N16 homoeologous chromosomal reciprocal translocation into a progeny canola plant is detected by producing an amplicon of SEQ ID NO:1, wherein the amplicon comprises a primer associated with any one of SEQ ID NO: 489-2990, SEQ ID NO:5 SEQ ID NO:6, SEQ ID NO:13, SEQ ID NO:15 or a complement thereof. Accordingly, the amplicon of SEQ ID NO:1 comprises a forward or 5′ primer associated with any one of SEQ ID NO: 489-2990, SEQ ID NO:5 SEQ ID NO:6, SEQ ID NO:13, SEQ ID NO:15 or a complement thereof. Likewise, the amplicon of SEQ ID NO:1 comprises a reverse or 3′ primer associated with any one of SEQ ID NO: 489-2990, SEQ ID NO:5 SEQ ID NO:6, SEQ ID NO:13, SEQ ID NO:15 or a complement thereof. In some aspects, the amplicon of SEQ ID NO:1 comprises a probe associated with any one of SEQ ID NO:138-254 or a complement thereof. In further aspects, the PCR assay to determine zygosity of the N7/N16 homoeologous chromosomal reciprocal translocation into a progeny canola plant is detected by producing an amplicon of SEQ ID NO:3, wherein the amplicon comprises a primer associated with any one of SEQ ID NO: 2991-5492, SEQ ID NO:7, SEQ ID NO:8, SEQ ID NO:17, SEQ ID NO:19 or a complement thereof. Accordingly, the amplicon of SEQ ID NO:3 comprises a forward or 5′ primer associated with any one of SEQ ID NO: 2991-5492, SEQ ID NO:7, SEQ ID NO:8, SEQ ID NO:17, SEQ ID NO:19 or a complement thereof. Likewise, the amplicon of SEQ ID NO:3 comprises a reverse or 3′ primer associated with any one of SEQ ID NO: 2991-5492, SEQ ID NO:7, SEQ ID NO:8, SEQ ID NO:17, SEQ ID NO:19 or a complement thereof. In other aspects, the amplicon of SEQ ID NO:3 comprises a probe associated with any one of SEQ ID NO: 21-137 or a complement thereof. In further aspects, the amplicon of SEQ ID NO:1 comprises a polynucleotide that shares at least 98% sequence identity with a primer associated with any one of SEQ ID NO: 489-2990, SEQ ID NO:5 SEQ ID NO:6, SEQ ID NO:13, SEQ ID NO:15 or a complement thereof. In other aspects, the amplicon of SEQ ID NO:1 comprises a polynucleotide that shares at least 95%, 96%, 97%, 98%, or 99% sequence identity with a primer associated with any one of SEQ ID NO: 489-2990, SEQ ID NO:5 SEQ ID NO:6, SEQ ID NO:13, SEQ ID NO:15 or a complement thereof. In an aspect, the amplicon of SEQ ID NO:1 comprises a probe that shares at least 95%, 96%, 97%, 98%, or 99% sequence identity with a probe associated with any one of SEQ ID NO:138-254 or a complement thereof. In some aspects, the amplicon of SEQ ID NO:3 comprises a polynucleotide that shares at least 98% sequence identity with a primer associated with any one of SEQ ID NO: 2991-5492, SEQ ID NO:7, SEQ ID NO:8, SEQ ID NO:17, SEQ ID NO:19 or a complement thereof. In further aspects, the amplicon of SEQ ID NO:3 comprises a polynucleotide that shares at least 95%, 96%, 97%, 98%, or 99% sequence identity with a primer associated with any one SEQ ID NO: 2991-5492, SEQ ID NO:7, SEQ ID NO:8, SEQ ID NO:17, SEQ ID NO:19 or a complement thereof. In a further aspect, the amplicon of SEQ ID NO:3 comprises a probe that shares at least 95%, 96%, 97%, 98%, or 99% sequence identity with a probe associated with any one of SEQ ID NO: 21-137 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In an aspect, the subject disclosure relates to introgressing a N7/N16 homoeologous chromosomal reciprocal translocation into canola germplasm. In a further aspect, the disclosures relates to detecting in a first canola plant at least one at least one PCR amplicon associated with the N7/N16 homoeologous chromosomal reciprocal translocation, wherein the one or more PCR amplicons localize within either SEQ ID NO:1, SEQ ID NO:2, SEQ ID NO:3, or SEQ ID NO:4. In another aspect, the disclosure relates to introgressing the N7/N16 homoeologous chromosomal reciprocal translocation into canola germplasm. In an aspect, disclosure relates to introgressing a trait within 1 megabase pairs, 950 kilobase pairs, 900 kilobase pairs, 850 kilobase pairs, 800 kilobase pairs, 750 kilobase pairs, 700 kilobase pairs, 650 kilobase pairs, 600 kilobase pairs, 550 kilobase pairs, 500 kilobase pairs, 450 kilobase pairs, 400 kilobase pairs, 350 kilobase pairs, 300 kilobase pairs, 250 kilobase pairs, 200 kilobase pairs, 150 kilobase pairs, 100 kilobase pairs, 90 kilobase pairs, 80 kilobase pairs, 70 kilobase pairs, 60 kilobase pairs, 50 kilobase pairs, 40 kilobase pairs, 30 kilobase pairs, 20 kilobase pairs, 10 kilobase pairs, 9 kilobase pairs, 8 kilobase pairs, 7 kilobase pairs, 6 kilobase pairs, 5 kilobase pairs, 4 kilobase pairs, 3 kilobase pairs, 2 kilobase pairs, 1 kilobase pairs, 750 base pairs, or 500 base pairs of SEQ ID NO:2 or SEQ ID NO:4. In a further aspect, disclosure relates to introgressing a trait within 1 megabase pairs, 950 kilobase pairs, 900 kilobase pairs, 850 kilobase pairs, 800 kilobase pairs, 750 kilobase pairs, 700 kilobase pairs, 650 kilobase pairs, 600 kilobase pairs, 550 kilobase pairs, 500 kilobase pairs, 450 kilobase pairs, 400 kilobase pairs, 350 kilobase pairs, 300 kilobase pairs, 250 kilobase pairs, 200 kilobase pairs, 150 kilobase pairs, 100 kilobase pairs, 90 kilobase pairs, 80 kilobase pairs, 70 kilobase pairs, 60 kilobase pairs, 50 kilobase pairs, 40 kilobase pairs, 30 kilobase pairs, 20 kilobase pairs, 10 kilobase pairs, 9 kilobase pairs, 8 kilobase pairs, 7 kilobase pairs, 6 kilobase pairs, 5 kilobase pairs, 4 kilobase pairs, 3 kilobase pairs, 2 kilobase pairs, 1 kilobase pairs, 750 base pairs, or 500 base pairs of SEQ ID NO:1 or SEQ ID NO:3. In other aspects, the trait is selected from the group consisting of an insecticidal resistance trait, a herbicide tolerance trait, a nitrogen use efficiency trait, a water use efficiency trait, a nutritional quality trait, a DNA binding trait, a selectable marker trait, a blackleg resistance trait, a Fusarium wilt trait, a White Rust tolerance trait, a lodging resistance trait, a plant height trait, an earliness of maturity trait, an oil trait, a protein trait, and a glucosinolate trait.

In some aspects the disclosure relates to a PCR assay method for detecting the trait that is introgressed within 1 megabase pairs, 950 kilobase pairs, 900 kilobase pairs, 850 kilobase pairs, 800 kilobase pairs, 750 kilobase pairs, 700 kilobase pairs, 650 kilobase pairs, 600 kilobase pairs, 550 kilobase pairs, 500 kilobase pairs, 450 kilobase pairs, 400 kilobase pairs, 350 kilobase pairs, 300 kilobase pairs, 250 kilobase pairs, 200 kilobase pairs, 150 kilobase pairs, 100 kilobase pairs, 90 kilobase pairs, 80 kilobase pairs, 70 kilobase pairs, 60 kilobase pairs, 50 kilobase pairs, 40 kilobase pairs, 30 kilobase pairs, 20 kilobase pairs, 10 kilobase pairs, 9 kilobase pairs, 8 kilobase pairs, 7 kilobase pairs, 6 kilobase pairs, 5 kilobase pairs, 4 kilobase pairs, 3 kilobase pairs, 2 kilobase pairs, 1 kilobase pairs, 750 base pairs, or 500 base pairs of a N7/N16 homoeologous chromosomal reciprocal translocation in a canola plant, tissue, or cell, wherein PCR assay occurs within either SEQ ID NO:2 or SEQ ID NO:4. In further aspects, the PCR assay of the N7/N16 homoeologous chromosomal reciprocal translocation into a progeny canola plant is detected by producing an amplicon of SEQ ID NO:2, wherein the amplicon comprises a primer associated with any one of SEQ ID NO:5493-8026, SEQ ID NO:9, SEQ ID NO:10, SEQ ID NO:14, SEQ ID NO:16 or a complement thereof. Accordingly, the amplicon of SEQ ID NO:2 comprises a forward or 5′ primer associated with any one of SEQ ID NO:5493-8026, SEQ ID NO:9, SEQ ID NO:10, SEQ ID NO:14, SEQ ID NO:16 or a complement thereof. Likewise, the amplicon of SEQ ID NO:2 comprises a reverse or 3′ primer associated with any one of SEQ ID NO:5493-8026, SEQ ID NO:9, SEQ ID NO:10, SEQ ID NO:14, SEQ ID NO:16 or a complement thereof. In some aspects, the amplicon of SEQ ID NO:2 comprises a probe associated with any one of SEQ ID NO:372-488 or a complement thereof. In other aspects, the PCR assay of the N7/N16 homoeologous chromosomal reciprocal translocation in a canola plant is detected by producing an amplicon of SEQ ID NO:4, wherein the amplicon comprises a primer associated with any one of SEQ ID NO: 8027-10560, SEQ ID NO:11, SEQ ID NO:12, SEQ ID NO:18, SEQ ID NO:20 or a complement thereof. Accordingly, the amplicon of SEQ ID NO:4 comprises a forward or 5′ primer associated with any one of SEQ ID NO: 8027-10560, SEQ ID NO:11, SEQ ID NO:12, SEQ ID NO:18, SEQ ID NO:20 or a complement thereof. Likewise, the amplicon of SEQ ID NO:4 comprises a reverse or 3′ primer associated with any one of SEQ ID NO: 8027-10560, SEQ ID NO:11, SEQ ID NO:12, SEQ ID NO:18, SEQ ID NO:20 or a complement thereof. In other aspects, the amplicon of SEQ ID NO:4 comprises a probe associated with any one of SEQ ID NO:255-371 or a complement thereof. In further aspects, the amplicon of SEQ ID NO:2 comprises a polynucleotide that shares at least 98% sequence identity with a primer associated with any one of SEQ ID NO:5493-8026, SEQ ID NO:9, SEQ ID NO:10, SEQ ID NO:14, SEQ ID NO:16 or a complement thereof. In other aspects, the amplicon of SEQ ID NO:2 comprises a polynucleotide that shares at least 95%, 96%, 97%, 98%, or 99% sequence identity with a primer associated with any one of SEQ ID NO:5493-8026, SEQ ID NO:9, SEQ ID NO:10, SEQ ID NO:14, SEQ ID NO:16 or a complement thereof. In an aspect, the amplicon of SEQ ID NO:2 comprises a probe that shares at least 95%, 96%, 97%, 98%, or 99% sequence identity with a probe associated with any one of SEQ ID NO:372-488 or a complement thereof. In some aspects, the amplicon of SEQ ID NO:4 comprises a polynucleotide that shares at least 98% sequence identity with a primer associated with any one of SEQ ID NO: 8027-10560, SEQ ID NO:11, SEQ ID NO:12, SEQ ID NO:18, SEQ ID NO:20 or a complement thereof. In further aspects, the amplicon of SEQ ID NO:4 comprises a polynucleotide that shares at least 95%, 96%, 97%, 98%, or 99% sequence identity with a primer associated with any one SEQ ID NO: 8027-10560, SEQ ID NO: 11, SEQ ID NO:12, SEQ ID NO:18, SEQ ID NO:20 or a complement thereof. In a further aspect, the amplicon of SEQ ID NO:4 comprises a probe that shares at least 95%, 96%, 97%, 98%, or 99% sequence identity with a probe associated with any one of SEQ ID NO:255-371 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In some aspects the disclosure relates to a PCR assay method for detecting the trait that is introgressed within 1 megabase pairs, 950 kilobase pairs, 900 kilobase pairs, 850 kilobase pairs, 800 kilobase pairs, 750 kilobase pairs, 700 kilobase pairs, 650 kilobase pairs, 600 kilobase pairs, 550 kilobase pairs, 500 kilobase pairs, 450 kilobase pairs, 400 kilobase pairs, 350 kilobase pairs, 300 kilobase pairs, 250 kilobase pairs, 200 kilobase pairs, 150 kilobase pairs, 100 kilobase pairs, 90 kilobase pairs, 80 kilobase pairs, 70 kilobase pairs, 60 kilobase pairs, 50 kilobase pairs, 40 kilobase pairs, 30 kilobase pairs, 20 kilobase pairs, 10 kilobase pairs, 9 kilobase pairs, 8 kilobase pairs, 7 kilobase pairs, 6 kilobase pairs, 5 kilobase pairs, 4 kilobase pairs, 3 kilobase pairs, 2 kilobase pairs, 1 kilobase pairs, 750 base pairs, or 500 base pairs of a N7/N16 homoeologous chromosomal reciprocal translocation in a canola plant, tissue, or cell, wherein PCR assay occurs within either SEQ ID NO:1 or SEQ ID NO:3. In further aspects, the PCR assay of the N7/N16 homoeologous chromosomal reciprocal translocation into a progeny canola plant is detected by producing an amplicon of SEQ ID NO:1, wherein the amplicon comprises a primer associated with any one of SEQ ID NO: 489-2990, SEQ ID NO:5 SEQ ID NO:6, SEQ ID NO:13, SEQ ID NO:15 or a complement thereof. Accordingly, the amplicon of SEQ ID NO:1 comprises a forward or 5′ primer associated with any one of SEQ ID NO: 489-2990, SEQ ID NO:5 SEQ ID NO:6, SEQ ID NO:13, SEQ ID NO:15 or a complement thereof. Likewise, the amplicon of SEQ ID NO:1 comprises a reverse or 3′ primer associated with any one of SEQ ID NO: 489-2990, SEQ ID NO:5 SEQ ID NO:6, SEQ ID NO:13, SEQ ID NO:15 or a complement thereof. In some aspects, the amplicon of SEQ ID NO:1 comprises a probe associated with any one of SEQ ID NO:138-254 or a complement thereof. In further aspects, the PCR assay is detected by producing an amplicon of SEQ ID NO:3, wherein the amplicon comprises a primer associated with any one of SEQ ID NO: 2991-5492, SEQ ID NO:7, SEQ ID NO:8, SEQ ID NO:17, SEQ ID NO:19 or a complement thereof. Accordingly, the amplicon of SEQ ID NO:3 comprises a forward or 5′ primer associated with any one of SEQ ID NO: 2991-5492, SEQ ID NO:7, SEQ ID NO:8, SEQ ID NO:17, SEQ ID NO:19 or a complement thereof. Likewise, the amplicon of SEQ ID NO:3 comprises a reverse or 3′ primer associated with any one of SEQ ID NO: 2991-5492, SEQ ID NO:7, SEQ ID NO:8, SEQ ID NO:17, SEQ ID NO:19 or a complement thereof. In other aspects, the amplicon of SEQ ID NO:3 comprises a probe associated with any one of SEQ ID NO: 21-137 or a complement thereof. In further aspects, the amplicon of SEQ ID NO:1 comprises a polynucleotide that shares at least 98% sequence identity with a primer associated with any one of SEQ ID NO: 489-2990, SEQ ID NO:5 SEQ ID NO:6, SEQ ID NO:13, SEQ ID NO:15 or a complement thereof. In other aspects, the amplicon of SEQ ID NO:1 comprises a polynucleotide that shares at least 95%, 96%, 97%, 98%, or 99% sequence identity with a primer associated with any one of SEQ ID NO: 489-2990, SEQ ID NO:5 SEQ ID NO:6, SEQ ID NO:13, SEQ ID NO:15 or a complement thereof. In an aspect, the amplicon of SEQ ID NO:1 comprises a probe that shares at least 95%, 96%, 97%, 98%, or 99% sequence identity with a probe associated with any one of SEQ ID NO:138-254 or a complement thereof. In some aspects, the amplicon of SEQ ID NO:3 comprises a polynucleotide that shares at least 98% sequence identity with a primer associated with any one of SEQ ID NO: 2991-5492, SEQ ID NO:7, SEQ ID NO:8, SEQ ID NO:17, SEQ ID NO:19 or a complement thereof. In further aspects, the amplicon of SEQ ID NO:3 comprises a polynucleotide that shares at least 95%, 96%, 97%, 98%, or 99% sequence identity with a primer associated with any one SEQ ID NO: 2991-5492, SEQ ID NO:7, SEQ ID NO:8, SEQ ID NO:17, SEQ ID NO:19 or a complement thereof. In a further aspect, the amplicon of SEQ ID NO:3 comprises a probe that shares at least 95%, 96%, 97%, 98%, or 99% sequence identity with a probe associated with any one of SEQ ID NO: 21-137 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In another aspect, the disclosure relates to selecting a canola plant comprising an N7/N16 homoeologous chromosomal reciprocal translocation. In an aspect, the disclosure relates to genotyping at least one canola plant with respect to the N7/N16 homoeologous chromosomal reciprocal translocation, wherein the at least one canola plant comprises SEQ ID NO:255-488 or a complement thereof. In a further aspect, the disclosure relates to selecting a canola plant that includes the N7/N16 homoeologous chromosomal reciprocal translocation associated with SEQ ID NO:255-488 or a complement thereof. In a further aspect, the disclosure relates to a PCR amplicon comprising SEQ ID NO:255-488. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In another aspect, the disclosure relates to selecting a canola plant comprising an N7/N16 homoeologous chromosomal reciprocal translocation. In an aspect, the disclosure relates to genotyping at least one canola plant with respect to the N7/N16 homoeologous chromosomal reciprocal translocation, wherein the at least one canola plant comprises SEQ ID NO:21-254 or a complement thereof. In a further aspect, the disclosure relates to selecting a canola plant that includes the N7/N16 homoeologous chromosomal reciprocal translocation associated with SEQ ID NO:21-254 or a complement thereof. In a further aspect, the disclosure relates to a PCR amplicon comprising SEQ ID NO:21-254. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In another aspect, the disclosure relates to a PCR assay method for determining the recombinant genetic frequencies in canola plants comprising the N7/N16 homoeologous chromosomal reciprocal translocation. In some aspects, the recombinant genetic frequency of the N7/N16 homoeologous chromosomal reciprocal translocation is determined in a canola plant, tissue, or cell. In some aspects, the disclosure relates to performing a first PCR assay using a first probe, a first forward primer, and a first reverse primer on a polynucleotide from a canola plant sample. In other aspects, the disclosure relates to performing a second PCR assay using a second probe, a second forward primer, and a second reverse primer on the polynucleotide sample. In further aspects, the disclosure relates to quantifying the first probe and the second probe. In additional aspect, the disclosure relates to comparing the quantified first probe and the quantified second probe of the first PCR assay and the second PCR assay to determine the recombinant genetic frequencies, wherein the recombinant genetic frequency of the N7/N16 homoeologous chromosomal reciprocal translocation is selected from the group consisting of linked, tightly linked, or extremely tightly linked. In an aspect, the first and the second PCR assays are a multiplex PCR-format. In another aspect, the first and second PCR assays are performed in a single PCR assay tube or well. In an additional aspect, the first and the second PCR assays are a real-time PCR. In other aspects, the first probe and the second probe are quantified by measuring excitation/emission spectra emitted from the fluorescent dyes, during the amplification. In further aspects, the zygosity is determined by comparing the quantified first probe and the second probe using a ΔΔCt formula.

In some aspects the disclosure relates to a PCR assay method for determining the recombinant genetic frequency of a N7/N16 homoeologous chromosomal reciprocal translocation in a canola plant, tissue, or cell, wherein PCR assay occurs within either SEQ ID NO:2 or SEQ ID NO:4. In further aspects, the PCR assay of the N7/N16 homoeologous chromosomal reciprocal translocation into a progeny canola plant is detected by producing an amplicon of SEQ ID NO:2, wherein the amplicon comprises a primer associated with any one of SEQ ID NO:5493-8026, SEQ ID NO:9, SEQ ID NO:10, SEQ ID NO:14, SEQ ID NO:16 or a complement thereof. Accordingly, the amplicon of SEQ ID NO:2 comprises a forward or 5′ primer associated with any one of SEQ ID NO:5493-8026, SEQ ID NO:9, SEQ ID NO:10, SEQ ID NO:14, SEQ ID NO:16 or a complement thereof. Likewise, the amplicon of SEQ ID NO:2 comprises a reverse or 3′ primer associated with any one of SEQ ID NO:5493-8026, SEQ ID NO:9, SEQ ID NO:10, SEQ ID NO:14, SEQ ID NO:16 or a complement thereof. In some aspects, the amplicon of SEQ ID NO:2 comprises a probe associated with any one of SEQ ID NO:372-488 or a complement thereof. In other aspects, the PCR assay method of a N7/N16 homoeologous chromosomal reciprocal translocation in a canola plant is detected by producing an amplicon of SEQ ID NO:4, wherein the amplicon comprises a primer associated with any one of SEQ ID NO: 8027-10560, SEQ ID NO: 11, SEQ ID NO:12, SEQ ID NO:18, SEQ ID NO:20 or a complement thereof. Accordingly, the amplicon of SEQ ID NO:4 comprises a forward or 5′ primer associated with any one of SEQ ID NO: 8027-10560, SEQ ID NO:11, SEQ ID NO:12, SEQ ID NO:18, SEQ ID NO:20 or a complement thereof. Likewise, the amplicon of SEQ ID NO:4 comprises a reverse or 3′ primer associated with any one of SEQ ID NO: 8027-10560, SEQ ID NO:11, SEQ ID NO:12, SEQ ID NO:18, SEQ ID NO:20 or a complement thereof. In other aspects, the amplicon of SEQ ID NO:4 comprises a probe associated with any one of SEQ ID NO:255-371 or a complement thereof. In further aspects, the amplicon of SEQ ID NO:2 comprises a polynucleotide that shares at least 98% sequence identity with a primer associated with any one of SEQ ID NO:5493-8026, SEQ ID NO:9, SEQ ID NO:10, SEQ ID NO:14, SEQ ID NO:16 or a complement thereof. In other aspects, the amplicon of SEQ ID NO:2 comprises a polynucleotide that shares at least 95%, 96%, 97%, 98%, or 99% sequence identity with a primer associated with any one of SEQ ID NO:5493-8026, SEQ ID NO:9, SEQ ID NO:10, SEQ ID NO:14, SEQ ID NO:16 or a complement thereof. In an aspect, the amplicon of SEQ ID NO:2 comprises a probe that shares at least 95%, 96%, 97%, 98%, or 99% sequence identity with a probe associated with any one of SEQ ID NO:372-488 or a complement thereof. In some aspects, the amplicon of SEQ ID NO:4 comprises a polynucleotide that shares at least 98% sequence identity with a primer associated with any one of SEQ ID NO: 8027-10560, SEQ ID NO:11, SEQ ID NO:12, SEQ ID NO:18, SEQ ID NO:20 or a complement thereof. In further aspects, the amplicon of SEQ ID NO:4 comprises a polynucleotide that shares at least 95%, 96%, 97%, 98%, or 99% sequence identity with a primer associated with any one SEQ ID NO: 8027-10560, SEQ ID NO: 11, SEQ ID NO:12, SEQ ID NO:18, SEQ ID NO:20 or a complement thereof. In a further aspect, the amplicon of SEQ ID NO:4 comprises a probe that shares at least 95%, 96%, 97%, 98%, or 99% sequence identity with a probe associated with any one of SEQ ID NO:255-371 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

In some aspects the disclosure relates to a PCR assay method for determining the recombinant genetic frequency of a N7/N16 homoeologous chromosomal reciprocal translocation in a canola plant, tissue, or cell, wherein the PCR assay occurs within either SEQ ID NO:1 or SEQ ID NO:3. In further aspects, the PCR assay of the N7/N16 homoeologous chromosomal reciprocal translocation into a progeny canola plant is detected by producing an amplicon of SEQ ID NO:1, wherein the amplicon comprises a primer associated with any one of SEQ ID NO: 489-2990, SEQ ID NO:5 SEQ ID NO:6, SEQ ID NO:13, SEQ ID NO:15 or a complement thereof. Accordingly, the amplicon of SEQ ID NO:1 comprises a forward or 5′ primer associated with any one of SEQ ID NO: 489-2990, SEQ ID NO:5 SEQ ID NO:6, SEQ ID NO:13, SEQ ID NO:15 or a complement thereof. Likewise, the amplicon of SEQ ID NO:1 comprises a reverse or 3′ primer associated with any one of SEQ ID NO: 489-2990, SEQ ID NO:5 SEQ ID NO:6, SEQ ID NO:13, SEQ ID NO:15 or a complement thereof. In some aspects, the amplicon of SEQ ID NO:1 comprises a probe associated with any one of SEQ ID NO:138-254 or a complement thereof. In further aspects, the PCR assay of the N7/N16 homoeologous chromosomal reciprocal translocation into a progeny canola plant is detected by producing an amplicon of SEQ ID NO:3, wherein the amplicon comprises a primer associated with any one of SEQ ID NO: 2991-5492, SEQ ID NO:7, SEQ ID NO:8, SEQ ID NO:17, SEQ ID NO:19 or a complement thereof. Accordingly, the amplicon of SEQ ID NO:3 comprises a forward or 5′ primer associated with any one of SEQ ID NO: 2991-5492, SEQ ID NO:7, SEQ ID NO:8, SEQ ID NO:17, SEQ ID NO:19 or a complement thereof. Likewise, the amplicon of SEQ ID NO:3 comprises a reverse or 3′ primer associated with any one of SEQ ID NO: 2991-5492, SEQ ID NO:7, SEQ ID NO:8, SEQ ID NO:17, SEQ ID NO:19 or a complement thereof. In other aspects, the amplicon of SEQ ID NO:3 comprises a probe associated with any one of SEQ ID NO: 21-137 or a complement thereof. In further aspects, the amplicon of SEQ ID NO:1 comprises a polynucleotide that shares at least 98% sequence identity with a primer associated with any one of SEQ ID NO: 489-2990, SEQ ID NO:5 SEQ ID NO:6, SEQ ID NO:13, SEQ ID NO:15 or a complement thereof. In other aspects, the amplicon of SEQ ID NO:1 comprises a polynucleotide that shares at least 95%, 96%, 97%, 98%, or 99% sequence identity with a primer associated with any one of SEQ ID NO: 489-2990, SEQ ID NO:5 SEQ ID NO:6, SEQ ID NO:13, SEQ ID NO:15 or a complement thereof. In an aspect, the amplicon of SEQ ID NO:1 comprises a probe that shares at least 95%, 96%, 97%, 98%, or 99% sequence identity with a probe associated with any one of SEQ ID NO:138-254 or a complement thereof. In some aspects, the amplicon of SEQ ID NO:3 comprises a polynucleotide that shares at least 98% sequence identity with a primer associated with any one of SEQ ID NO: 2991-5492, SEQ ID NO:7, SEQ ID NO:8, SEQ ID NO:17, SEQ ID NO:19 or a complement thereof. In further aspects, the amplicon of SEQ ID NO:3 comprises a polynucleotide that shares at least 95%, 96%, 97%, 98%, or 99% sequence identity with a primer associated with any one SEQ ID NO: 2991-5492, SEQ ID NO:7, SEQ ID NO:8, SEQ ID NO:17, SEQ ID NO:19 or a complement thereof. In a further aspect, the amplicon of SEQ ID NO:3 comprises a probe that shares at least 95%, 96%, 97%, 98%, or 99% sequence identity with a probe associated with any one of SEQ ID NO: 21-137 or a complement thereof. In an additional aspect, the disclosure relates to quantitating the amplicon produced by an amplification reaction. In some aspects, quantitating the results of the amplification reaction comprises producing a signature profile. As such, the signature profile is selected from the group consisting of a melting temperature curve signature profile and a fluorescence signature profile. Further, the signature profile is produced from an intercalating DNA dye, a cyanine dye, or a fluorescent dye. Likewise, the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. In yet a further aspect, the disclosure relates to determining the presence or absence of the N7/N16 homoeologous chromosomal reciprocal translocation by determining the nucleotide sequence of said nucleic acid. In another aspect, the disclosure relates to determining the presence or absence of the N7/N16 homoeologous chromosomal reciprocal translocation by determining the size of said nucleic acid. In an aspect, determining the size comprises HPLC or electrophoresis.

The foregoing and other features will become more apparent from the following embodiments as provided in the Claims and Detailed Description, which proceeds with reference to the accompanying FIGURES and Sequence Listing.

### SEQUENCE LISTING

The nucleic acid sequences listed in the accompanying sequence listing are shown using standard letter abbreviations for nucleotide bases, as defined in 37 C.F.R. § 1.822. Only one strand of each nucleic acid sequence is shown, but the complementary strand and reverse complementary strand are understood as included by any reference to the displayed strand. As the complement and reverse complement of a primary nucleic acid sequence are necessarily disclosed by the primary sequence, the complementary sequence and reverse complementary sequence of a nucleic acid sequence are included by any reference to the nucleic acid sequence, unless it is explicitly stated to be otherwise (or it is clear to be otherwise from the context in which the sequence appears). In the accompanying sequence listing:

The chromosomal interval of base pairs 17578019 to 17583020 of chromosome 7 from the DH12075 v1.1 genome is provided as SEQ ID NO:1.

The chromosomal interval of base pairs 20106368 to 20111369 of chromosome 7 from the NS1822BC genome is provided as SEQ ID NO:2.

The chromosomal interval of base pairs 13651438 to 13656439 of chromosome 16 from the DH12075 v1.1 genome is provided as SEQ ID NO:3.

The chromosomal interval of base pairs 8089982 to 8094983 of chromosome 16 from the NS1822BC genome is provided as SEQ ID NO:4.

The DHN16 probe sequences are provided as SEQ ID NO: 21-137.

The DHN7 probe sequences are provided as SEQ ID NO: 138-254.

The NSN16 probe sequences are provided as SEQ ID NO: 255-371.

The NSN7 probe sequences are provided as SEQ ID NO: 372-488.

The DHN7 primer sequences are provided as SEQ ID NO: 489-2990, SEQ ID NO:5 SEQ ID NO:6, SEQ ID NO:13 and SEQ ID NO:15.

The DHN16 primer sequences are provided as SEQ ID NO: 2991-5492, SEQ ID NO:7, SEQ ID NO:8, SEQ ID NO:17 and SEQ ID NO:19.

The NSN7 primer sequences are provided as SEQ ID NO: 5493-8026, SEQ ID NO:9, SEQ ID NO:10, SEQ ID NO:14 and SEQ ID NO:16.

The NSN16 primer sequences are provided as SEQ ID NO: 8027-10560, SEQ ID NO:11, SEQ ID NO:12, SEQ ID NO:18 and SEQ ID NO:20.

### DETAILED DESCRIPTION

Development of novel methods and compositions for molecular breeding assays that can be readily applied for breeding in B. napus are of importance for B. napus breeders. Commercially viable assays that are time efficient and can provide cost saving are of particular interest. Of further importance is the ability to complete the assay in a variety of different scales. From a single Eppendorf™ tube to larger multi-well formats, the application of the assay should be readily utilized in any applied format. Methods and compositions for molecular breeding assays that can detect a N7/N16 homoeologous chromosomal reciprocal translocation in B. napus are disclosed herein. A detailed genomic characterization of the break point, the genetic/physical extent of this homoeologous chromosomal reciprocal translocation, frequency in the global elite germplasm & distribution in various heterotic pools, and linkage with traits of commercial value are provided for the first time in this disclosure.

The methods and compositions for molecular breeding assays that can detect a N7/N16 homoeologous chromosomal reciprocal translocation in B. napus of the subject disclosure provide markable improvements over the low throughput, low resolution and expensive methods (e.g., Fluorescent In Situ Hybridization (FISH), and use of low-density molecular marker systems such as RFLP and SSR, and other known methods) that are generally available for routing breeding. The presence of the N7/N16 homoeologous chromosomal reciprocal translocation in B. napus in breeding progenies results in non-parental recombinant types, characterized by segmental deletion and duplication in homoeologous regions. The methods and compositions for molecular breeding assays that can detect a N7/N16 homoeologous chromosomal reciprocal translocation in B. napus provide numerous advantages to overcome particular problems for B. napus breeding.

A particular problem for B. napus breeders is the development of an assay to detect the introgression of traits in B. napus breeding programs. Traits that are bred into proximity of the N7/N16 homoeologous chromosomal reciprocal translocation in B. napus can be introduced into new lines of B. napus germplasm through the translocation of the N7/N16 homoeologous chromosomal. Either native traits or transgenic traits may be bred into loci within the chromosomal regions of either the N7 or N16 chromosome. Relying upon the translocation mechanism within specific lines of B. napus allows for the introgression of the trait into a new line of B. napus. As such, the methods and compositions for molecular breeding assays that can detect a N7/N16 homoeologous chromosomal reciprocal translocation in B. napus provide a solution for introgressing traits within B. napus.

A particular problem for B. napus breeders is the development of an assay to detect heterosis in B. napus breeding programs. The identification of “parental type” classes of B. napus lines among the breeding progenies that have been identified to segregate for N7/N16 homoeologous chromosomal reciprocal translocation in B. napus have heterotic advantage. Currently, there is no ability to detect and enrich/discard these variants leading to loss of cost and time efficiencies in breeding. As such, the methods and compositions for molecular breeding assays that can detect a N7/N16 homoeologous chromosomal reciprocal translocation in B. napus provide a solution for detecting heterosis within B. napus.

A particular problem for B. napus breeders is the development of an assay for signal detection in B. napus breeding programs. Typical commercial scale “signal detection” methods rely on a single reference genetic map and marker order, by-passing the construction of de novo population specific genetic map for signal detection. If a large homoeologous chromosomal reciprocal translocation, such as N7/N16, are differentially distributed/fixed in the heterotic pools, and the mapping populations are segregating for N7/N16 homoeologous chromosomal reciprocal translocation, then such signal detection assays could lead to inaccurate assignments of quantitative trait loci and genes present on these chromosomes, directly impacting marker driven forward and backcross breeding selections. As such, the methods and compositions for molecular breeding assays that can detect a N7/N16 homoeologous chromosomal reciprocal translocation in B. napus provide a solution for detecting the N7/N16 homoeologous chromosomal reciprocal translocation within B. napus.

Novel methods have now been disclosed for detecting the N7/N16 homoeologous chromosomal reciprocal translocations in B. napus. The disclosed methods can be deployed as high throughput assays allowing for the rapid and efficient identification of a subset of samples that can then be further processed through traditional plant breeding methodologies. The disclosed assays describe high quality, high throughput processes for identifying and obtaining the N7/N16 homoeologous chromosomal reciprocal translocations in B. napus.

### Definitions

Unless defined otherwise, all technical and scientific terms used herein have the same meaning as commonly understood by one of ordinary skill in the art to which this disclosure relates. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

In order to further clarify this disclosure, the following terms, abbreviations and definitions are provided.

As used herein, the terms “comprises”, “comprising”, “includes”, “including”, “has”, “having”, “contains”,” or “containing”, or any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of an embodiment of the disclosure are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as disclosed in the application.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques were designed primarily for this sorting out.

As used herein, the term “polymerase chain reaction” and “PCR” generally refers to the method for increasing the concentration of a segment of a target sequence in a mixture of genomic DNA without cloning or purification (U.S. Pat. Nos. 4,683,195; 4,683,202; and 4,965,188; herein incorporated by reference). This process for amplifying the target sequence comprises introducing an excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.

Term “amplicon” refers to as natural or manually amplification event (for example, polymerase chain reaction) product formation Polynucleotides (for example, DNA) segment. In some embodiments, amplicon is “length” amplicon, and the length is at least 5 kb, or At least 6 kb, 7 kb, 8 kb, 9 kb, 101b, 11 kb, 12 kb, 13 kb, 14 kb, 15 kb, 16 kb, 17 kb, 18 kb, 19 kb, or 20 kb.

The term “isolated”, as used herein means having been removed from its natural environment, or removed from other compounds present when the compound is first formed. The term “isolated” embraces materials isolated from natural sources as well as materials (e.g., nucleic acids and proteins) recovered after preparation by recombinant expression in a host cell, or chemically-synthesized compounds such as nucleic acid molecules, proteins, and peptides.

The term “purified”, as used herein relates to the isolation of a molecule or compound in a form that is substantially free of contaminants normally associated with the molecule or compound in a native or natural environment, or substantially enriched in concentration relative to other compounds present when the compound is first formed, and means having been increased in purity as a result of being separated from other components of the original composition. The term “purified nucleic acid” is used herein to describe a nucleic acid sequence which has been separated, produced apart from, or purified away from other biological compounds including, but not limited to polypeptides, lipids and carbohydrates, while effecting a chemical or functional change in the component (e.g., a nucleic acid may be purified from a chromosome by removing protein contaminants and breaking chemical bonds connecting the nucleic acid to the remaining DNA in the chromosome).

The term “synthetic”, as used herein refers to a polynucleotide (i.e., a DNA or RNA) molecule that was created via chemical synthesis as an in vitro process. For example, a synthetic DNA may be created during a reaction within an Eppendorf™ tube, such that the synthetic DNA is enzymatically produced from a native strand of DNA or RNA. Other laboratory methods may be utilized to synthesize a polynucleotide sequence. Oligonucleotides may be chemically synthesized on an oligo synthesizer via solid-phase synthesis using phosphoramidites. The synthesized oligonucleotides may be annealed to one another as a complex, thereby producing a “synthetic” polynucleotide. Other methods for chemically synthesizing a polynucleotide are known in the art, and can be readily implemented for use in the present disclosure.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

For the purposes of the present disclosure, a “gene,” includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, introns and locus control regions.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

As used herein a “transgene” is defined to be a nucleic acid sequence that encodes a gene product, including for example, but not limited to, an mRNA. In one embodiment the transgene/heterologous coding sequence is an exogenous nucleic acid, where the transgene/heterologous coding sequence has been introduced into a host cell by genetic engineering (or the progeny thereof) where the transgene/heterologous coding sequence is not normally found. In one example, a transgene/heterologous coding sequence encodes an industrially or pharmaceutically useful compound, or a gene encoding a desirable agricultural trait (e.g., an herbicide-resistance gene). In yet another example, a transgene/heterologous coding sequence is an antisense nucleic acid sequence, wherein expression of the antisense nucleic acid sequence inhibits expression of a target nucleic acid sequence. In one embodiment the transgene/heterologous coding sequence is an endogenous nucleic acid, wherein additional genomic copies of the endogenous nucleic acid are desired, or a nucleic acid that is in the antisense orientation with respect to the sequence of a target nucleic acid in a host organism.

A “gene product” as defined herein is any product produced by the gene. For example, the gene product can be the direct transcriptional product of a gene (e.g., mRNA, tRNA, rRNA, antisense RNA, interfering RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation. Gene expression can be influenced by external signals, for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

As used herein the term “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

As used herein, the term “nucleic acid molecule” (or “nucleic acid” or “polynucleotide”) may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide”. A nucleic acid molecule is usually at least 10 bases in length, unless otherwise specified. The term may refer to a molecule of RNA or DNA of indeterminate length. The term includes single- and double-stranded forms of DNA. A nucleic acid molecule may include either or both naturally-occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidites, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

Transcription proceeds in a 5′ to 3′ manner along a DNA strand. This means that RNA is made by the sequential addition of ribonucleotide-5′-triphosphates to the 3′ terminus of the growing chain (with a requisite elimination of the pyrophosphate). In either a linear or circular nucleic acid molecule, discrete elements (e.g., particular nucleotide sequences) may be referred to as being “upstream” or “5′” relative to a further element if they are bonded or would be bonded to the same nucleic acid in the 5′ direction from that element. Similarly, discrete elements may be “downstream” or “3′” relative to a further element if they are or would be bonded to the same nucleic acid in the 3′ direction from that element.

A base “position”, as used herein, refers to the location of a given base or nucleotide residue within a designated nucleic acid. The designated nucleic acid may be defined by alignment (see below) with a reference nucleic acid.

Hybridization relates to the binding of two polynucleotide strands via Hydrogen bonds. Oligonucleotides and their analogs hybridize by hydrogen bonding, which includes Watson-Crick, Hoogsteen or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide and the DNA or RNA target. The oligonucleotide need not be 100% complementary to its target sequence to be specifically hybridizable. An oligonucleotide is specifically hybridizable when binding of the oligonucleotide to the target DNA or RNA molecule interferes with the normal function of the target DNA or RNA, and there is sufficient degree of complementarity to avoid non-specific binding of the oligonucleotide to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the chosen hybridization method and the composition and length of the hybridizing nucleic acid sequences. Generally, the temperature of hybridization and the ionic strength (especially the Na+ and/or Mg2+ concentration) of the hybridization buffer will contribute to the stringency of hybridization, though wash times also influence stringency. Calculations regarding hybridization conditions required for attaining particular degrees of stringency are discussed in Sambrook et al. (ed.), Molecular Cloning: A Laboratory Manual, 2nd ed., vol. 1-3, Cold Spring Harbor Laboratory Press, Cold Spring Harbor, New York, 1989, chs. 9 and 11.

As used herein, “stringent conditions” encompass conditions under which hybridization will only occur if there is less than 50% mismatch between the hybridization molecule and the DNA target. “Stringent conditions” include further particular levels of stringency. Thus, as used herein, “moderate stringency” conditions are those under which molecules with more than 50% sequence mismatch will not hybridize; conditions of “high stringency” are those under which sequences with more than 20% mismatch will not hybridize; and conditions of “very high stringency” are those under which sequences with more than 10% mismatch will not hybridize.

In particular embodiments, stringent conditions can include hybridization at 65° C., followed by washes at 65° C. with 0.1×SSC/0.1% SDS for 40 minutes.

The following are representative, non-limiting hybridization conditions: Very High Stringency: Hybridization in 5×SSC buffer at 65° C. for 16 hours; was twice in 2×SSC buffer at room temperature for 15 minutes each; and wash twice in 0.5×SSC buffer at 65° C. for 20 minutes each. High Stringency: Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each. Moderate Stringency: Hybridization in 6×SSC buffer at room temperature to 55° C. for 16-20 hours; wash at least twice in 2×-3×SSC buffer at room temperature to 55° C. for 20-30 minutes each.

In particular embodiments, specifically hybridizable nucleic acid molecules can remain bound under very high stringency hybridization conditions. In these and further embodiments, specifically hybridizable nucleic acid molecules can remain bound under high stringency hybridization conditions. In these and further embodiments, specifically hybridizable nucleic acid molecules can remain bound under moderate stringency hybridization conditions.

As used herein, the term “oligonucleotide” refers to a short nucleic acid polymer. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of small DNA sequences. In PCR, the oligonucleotide is typically referred to as a “primer”, which allows a DNA polymerase to extend the oligonucleotide and replicate the complementary strand.

The terms “percent sequence identity” or “percent identity” or “identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

Global sequence alignment programs calculate the percent identity of two sequences by comparing alignments end-to-end in order to find exact matches, dividing the number of exact matches by the length of the shorter sequences, and then multiplying by 100. Basically, the percentage of identical nucleotides in a linear polynucleotide sequence of a reference (“query) polynucleotide molecule as compared to a test (“subject”) polynucleotide molecule when the two sequences are optimally aligned (with appropriate nucleotide insertions, deletions, or gaps).

Local sequence alignment programs are similar in their calculation, but only compare aligned fragments of the sequences rather than utilizing an end-to-end analysis. Local sequence alignment programs such as BLAST can be used to compare specific regions of two sequences. A BLAST comparison of two sequences results in an E-value, or expectation value, that represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST match is considered herein as having an E-value for the top BLAST hit of less than 1E-30; a medium BLASTX E-value is 1E-30 to 1E-8; and a low BLASTX E-value is greater than 1E-8. The protein function assignment in the present disclosure is determined using combinations of E-values, percent identity, query coverage and hit coverage. Query coverage refers to the percent of the query sequence that is represented in the BLAST alignment. Hit coverage refers to the percent of the database entry that is represented in the BLAST alignment. In one embodiment of the disclosure, function of a query polypeptide is inferred from function of a conserved protein sequence where either (1) hit_p<1e-30 or % identity >35% AND query_coverage >50% AND hit_coverage>50%, or (2) hit_p<1e-8 AND query_coverage>70% AND hit_coverage>70%.

Methods for aligning sequences for comparison are well-known in the art. Various programs and alignment algorithms are described. In an embodiment, the subject disclosure relates to calculating percent identity between two polynucleotides or amino acid sequences using an AlignX alignment program of the Vector NTI suite (Invitrogen, Carlsbad, CA). The AlignX alignment program is a global sequence alignment program for polynucleotides or proteins. In an embodiment, the subject disclosure relates to calculating percent identity between two polynucleotides or amino acid sequences using the MegAlign program of the LASERGENE bioinformatics computing suite (MegAlign™ (©1993-2016). DNASTAR. Madison, WI). The MegAlign program is global sequence alignment program for polynucleotides or proteins. In an embodiment, the subject disclosure relates to calculating percent identity between two polynucleotides or amino acid sequences using the Clustal suite of alignment programs, including, but not limited to, ClustalW and ClustalV (Higgins and Sharp (1988) Gene. December 15; 73(1):237-44; Higgins and Sharp (1989) CABIOS 5:151-3; Higgins et al. (1992) Comput. Appl. Biosci. 8:189-91). In an embodiment, the subject disclosure relates to calculating percent identity between two polynucleotides or amino acid sequences using the BLAST suite of alignment programs, for example, but not limited to, BLASTP, BLASTN, BLASTX, etc. (Altschul et al. (1990) J. Mol. Biol. 215:403-10). Further examples of such BLAST alignment programs include Gapped-BLAST or PSI-BLAST (Altschul et al., 1997). In an embodiment, the subject disclosure relates to calculating percent identity between two polynucleotides or amino acid sequences using the EMBOSS suite of alignment programs, including, but not limited to: Matcher, Needle, Stretcher, Water, Wordmatch, etc. (Rice, P., Longden, I. & Bleasby, A. EMBOSS: The European Molecular Biology Open Software Suite. Trends in Genetics 16(6) 276-77 (2000)). In an embodiment, the subject disclosure relates to calculating percent identity between two polynucleotides or amino acid sequences using the Gap alignment program of Needleman and Wunsch (Needleman and Wunsch, Journal of Molecular Biology 48:443-453, 1970). In an embodiment, the subject disclosure relates to calculating percent identity between two polynucleotides or amino acid sequences using the BestFit alignment program of Smith and Waterman (Smith and Waterman, Advances in Applied Mathematics, 2:482-489, 1981, Smith et al., Nucleic Acids Research 11:2205-2220, 1983). These programs produces biologically meaningful multiple sequence alignments of divergent sequences. The calculated best match alignments for the selected sequences are lined up so that identities, similarities, and differences can be seen.

The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

The term “homology” is sometimes used to refer to the level of similarity between two or more nucleic acid or amino acid sequences in terms of percent of positional identity (i.e., sequence similarity or identity). Homology also refers to the concept of evolutionary relatedness, often evidenced by similar functional properties among different nucleic acids or proteins that share similar sequences.

As used herein, the term “variants” means substantially similar sequences. For nucleotide sequences, naturally occurring variants can be identified with the use of well-known molecular biology techniques, such as, for example, with polymerase chain reaction (PCR) and hybridization techniques as outlined herein.

For nucleotide sequences, a variant comprises a deletion and/or addition of one or more nucleotides at one or more internal sites within the native polynucleotide and/or a substitution of one or more nucleotides at one or more sites in the native polynucleotide. As used herein, a “native” nucleotide sequence comprises a naturally occurring nucleotide sequence. For nucleotide sequences, naturally occurring variants can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis. Generally, variants of a particular nucleotide sequence of the disclosure will have at least about 40%, 45%, 50%>, 55%, 60%, 65%, 70%, 75%, 80%, 85%, 90%, 91%, 92%, 93%, 94%, 95%, 96%, 97%, 98%0, 99% or more sequence identity to that particular nucleotide sequence as determined by sequence alignment programs and parameters described elsewhere herein. A biologically active variant of a nucleotide sequence of the disclosure may differ from that sequence by as few as 1-15 nucleic acid residues, as few as 1-10, such as 6-10, as few as 5, as few as 4, 3, 2, or even 1 nucleic acid residue.

As used herein the term “operably linked” relates to a first nucleic acid sequence is operably linked with a second nucleic acid sequence when the first nucleic acid sequence is in a functional relationship with the second nucleic acid sequence. For instance, a promoter is operably linked with a coding sequence when the promoter affects the transcription or expression of the coding sequence. When recombinantly produced, operably linked nucleic acid sequences are generally contiguous and, where necessary to join two protein-coding regions, in the same reading frame. However, elements need not be contiguous to be operably linked.

As used herein, the term “regeneration” means the process of growing a plant from a plant cell (e.g., plant protoplast or explant).

As used herein, the term “culturing” refers to the in vitro propagation of cells or organisms on or in media of various kinds so that the maintenance or growth cell within a liquid culture medium are controlled under a set of physical conditions. It is understood that the descendants of a cell grown in culture may not be completely identical (i.e., morphologically, genetically, or phenotypically) to the parent cell.

As used herein, the term “co-expressing” refers to two or more gene products which are produced at the same time within the same host organism.

As used herein, the term “degenerate” refers to a primer or probe nucleic acid in which certain positions are not defined by a single, specific nucleotide. Thus, in such a degenerate position, the primer or probe sequence can be either one of at least two different nucleotides. Such positions often represent difference in genotypes of the target nucleic acid. A degenerate sequence may also be represented as a mixture of multiple non-degenerate individual sequences which, for the purpose of this disclosure, differ in at least two positions.

As used herein, the term “expression” refers to the combination of intracellular processes, including transcription and translation undergone by a coding DNA molecule such as a structural gene to produce a polypeptide.

As used herein, the term “transgenic cell” means any cell derived or regenerated from a transformed cell or derived from a transgenic cell. Exemplary transgenic cells include plant calli derived from a transformed plant cell and particular cells such as leaf, root, stem, e.g., somatic cells, or reproductive (germ) cells obtained from a transgenic plant.

As used herein, the term “transgenic plant” means a plant or progeny thereof derived from a transformed plant cell or protoplast, wherein the plant DNA contains an introduced exogenous DNA molecule not originally present in a native, non-transgenic plant of the same strain. The terms “transgenic plant” and “transformed plant” have sometimes been used in the art as synonymous terms to define a plant whose DNA contains an exogenous DNA molecule. However, it is thought more scientifically correct to refer to a regenerated plant or callus obtained from a transformed plant cell or protoplast as being a transgenic plant, and that usage will be followed herein.

As used herein, the term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is typically a single strand sequence, such that amplification efficiency is optimized, but double stranded sequences can be utilized.

As used herein, the term “probe” refers to an oligonucleotide that hybridizes to a target sequence. In the TaqMan® or TaqMan®-style assay procedure, the probe hybridizes to a portion of the target situated between the annealing site of the two primers. A probe includes about eight nucleotides, about ten nucleotides, about fifteen nucleotides, about twenty nucleotides, about thirty nucleotides, about forty nucleotides, or about fifty nucleotides. In some embodiments, a probe includes from about eight nucleotides to about fifteen nucleotides. A probe can further include a detectable label, e.g., a fluorophore (Texas-Red©, Fluorescein isothiocyanate, etc.,). The detectable label can be covalently attached directly to the probe oligonucleotide, e.g., located at the probe's 5′ end or at the probe's 3′ end. A probe including a fluorophore may also further include a quencher, e.g., Black Hole Quencher™, Iowa Black™, etc.

As used herein, the terms “restriction endonucleases” and “restriction enzymes” refer to bacterial enzymes, each of which cut double-stranded DNA at or near a specific nucleotide sequence. Type-2 restriction enzymes recognize and cleave DNA at the same site, and include but are not limited to XbaI, BamHI, HindIII, EcoRI, XhoI, SalI, KpnI, AvaI, PstI and SmaI.

As used herein, the term “vector” is used interchangeably with the terms “construct”, “cloning vector” and “expression vector” and means the vehicle by which a DNA or RNA sequence (e.g. a foreign gene) can be introduced into a host cell, so as to transform the host and promote expression (e.g. transcription and translation) of the introduced sequence. A “non-viral vector” is intended to mean any vector that does not comprise a virus or retrovirus. In some embodiments a “vector” is a sequence of DNA comprising at least one origin of DNA replication and at least one selectable marker gene. Examples include, but are not limited to, a plasmid, cosmid, bacteriophage, bacterial artificial chromosome (BAC), or virus that carries exogenous DNA into a cell. A vector can also include one or more genes, antisense molecules, and/or selectable marker genes and other genetic elements known in the art. A vector may transduce, transform, or infect a cell, thereby causing the cell to express the nucleic acid molecules and/or proteins encoded by the vector.

The term “plasmid” defines a circular strand of nucleic acid capable of autosomal replication in either a prokaryotic or a eukaryotic host cell. The term includes nucleic acid which may be either DNA or RNA and may be single- or double-stranded. The plasmid of the definition may also include the sequences which correspond to a bacterial origin of replication.

As used herein, the term “selectable marker gene” defines a gene or other expression cassette which encodes a protein which facilitates identification of cells into which the selectable marker gene is inserted. For example a “selectable marker gene” encompasses reporter genes as well as genes used in plant transformation to, for example, protect plant cells from a selective agent or provide resistance/tolerance to a selective agent. In one embodiment only those cells or plants that receive a functional selectable marker are capable of dividing or growing under conditions having a selective agent. The phrase “marker-positive” refers to plants that have been transformed to include a selectable marker gene.

As used herein, the term “detectable marker” refers to a label capable of detection, such as, for example, a radioisotope, fluorescent compound, bioluminescent compound, a chemiluminescent compound, metal chelator, or enzyme. Examples of detectable markers include, but are not limited to, the following: fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In an embodiment, a detectable marker can be attached by spacer arms of various lengths to reduce potential steric hindrance.

As used herein, the terms “cassette”, “expression cassette” and “gene expression cassette” refer to a segment of DNA that can be inserted into a nucleic acid or polynucleotide at specific restriction sites or by homologous recombination. As used herein the segment of DNA comprises a polynucleotide that encodes a polypeptide of interest, and the cassette and restriction sites are designed to ensure insertion of the cassette in the proper reading frame for transcription and translation. In an embodiment, an expression cassette can include a polynucleotide that encodes a polypeptide of interest and having elements in addition to the polynucleotide that facilitate transformation of a particular host cell. In an embodiment, a gene expression cassette may also include elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

As used herein a “linker” or “spacer” is a bond, molecule or group of molecules that binds two separate entities to one another. Linkers and spacers may provide for optimal spacing of the two entities or may further supply a labile linkage that allows the two entities to be separated from each other. Labile linkages include photocleavable groups, acid-labile moieties, base-labile moieties and enzyme-cleavable groups. The terms “polylinker” or “multiple cloning site” as used herein defines a cluster of three or more Type-2 restriction enzyme sites located within 10 nucleotides of one another on a nucleic acid sequence. In other instances the term “polylinker” as used herein refers to a stretch of nucleotides that are targeted for joining two sequences via any known seamless cloning method (i.e., Gibson Assembly®, NEBuilder HiFiDNA Assembly®, Golden Gate Assembly, BioBrick® Assembly, etc.). Constructs comprising a polylinker are utilized for the insertion and/or excision of nucleic acid sequences such as the coding region of a gene.

As used herein, the term “control” refers to a sample used in an analytical procedure for comparison purposes. A control can be “positive” or “negative”. For example, where the purpose of an analytical procedure is to detect a differentially expressed transcript or polypeptide in cells or tissue, it is generally preferable to include a positive control, such as a sample from a known plant exhibiting the desired expression, and a negative control, such as a sample from a known plant lacking the desired expression.

As used herein, the term “plant” includes a whole plant and any descendant, cell, tissue, or part of a plant. A class of plant that can be used in the present disclosure is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf, stem; fruit; seed; and root.

A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein.

As used herein, the term “linkage disequilibrium” refers to a statistical association between two loci or between a trait and a marker.

As used herein, linkage between genes or markers refers to the phenomenon in which genes or markers on a chromosome show a measurable probability of being passed on together to individuals in the next generation. The closer two genes or markers are to each other, the closer to (1) this probability becomes. Thus, the term “linked” may refer to one or more genes or markers that are passed together with a gene with a probability greater than 0.5 (which is expected from independent assortment where markers/genes are located on different chromosomes). Because the proximity of two genes or markers on a chromosome is directly related to the probability that the genes or markers will be passed together to individuals in the next generation, the term “linked” may also refer herein to one or more genes or markers that are located within about 2.0 Mb of one another on the same Brassica spp. chromosome. Thus, two “linked” genes or markers may be separated by about 2.1 Mb; 2.00 Mb; about 1.95 Mb; about 1.90 Mb; about 1.85 Mb; about 1.80 Mb; about 1.75 Mb; about 1.70 Mb; about 1.65 Mb; about 1.60 Mb; about 1.55 Nib; about 1.50 Mb, about 1.45 Mb; about 1.40 Mb; about 1.35 Mb; about 1.30 Mb; about 1.25 Mb; about 1.20 Mb; about 1.15 Mb; about 1.10 Mb; about 1.05 Mb; about 1.00 Mb; about 0.95 Mb; about 0.90 Mb; about 0.85 Mb; about 0.80 Mb; about 0.75 Mb; about 0.70 Mb; about 0.65 Mb; about 0.60 Nib; about 0.55 Mib; about 0.50 Nib; about 0.45 Nib; about 0.40 Mb; about 0.35 Mb; about 0.30 Mb; about 0.25 Mb; about 0.20 Mb; about 0.15 Mb; about 0.10 Mb; about 0.05 Mb; about 0.025 Mb; about 0.012 Mb; and about 0.01 Mb. A gene may be “linked” to a marker that resides within an exon or intron of the gene. In this case, the separation between the linked gene and marker is 0.00 Mb.

Markers and/or genes may also be “linked” to a phenotype, for example, a phenotype in which the linked gene or gene linked to the linked marker is involved. As will be understood by those of skill in the art, the length of this marker will vary if nucleotides are added or subtracted from the span of genomic DNA located between the distal ends of the particular primers used when annealed.

As used herein, the term “tightly linked” may refer to one or more genes or markers that are located within about 0.5 Mb of one another on the same chromosome. Thus, two “tightly linked” genes or markers may be separated by about 0.6 Mb; about 0.55 Mb; 0.5 Mb; about 0.45 Nib, about 0.4 Mb, about 0.35 Mb; about 0.3 Mb; about 0.25 Mb; about 0.2 Mb; about 0.15 Tb; about 0.12 Mb; about 0.1 Mb; about 0.05 Mb; and about 0.00 Mb.

As used herein, the term “extremely tightly linked” may refer to one or more genes or markers that are located within about 100 kb of one another on the same chromosome. Thus, two “extremely tightly linked” genes or markers may be separated by about 125 kb; about 120 kb; about 115 kb; about 110 kb; about 105 kb, 100 kb; about 95 kb; about 90 kb; about 85 kb; about 80 kb; about 75 kb; about 70 kb; about 65 kb; about 60 kb; about 55 kb; about 50 kb; about 45 kb; about 40 kb; about 35 kb; about 30 kb; about 25 kb; about 20 kb; about 15 kb; about 12 kb, about 10 kb; about 5 kb; about 1 kb; and about 0 kb.

Linked, tightly linked, and extremely tightly genetic markers may be useful in marker-assisted breeding programs to identify individuals comprising linked phenotypes and/or gene types, and to breed these traits and/or genes into Brassica varieties.

Additional markers can be identified as equivalent to this exemplary marker, for example, by determining the frequency of recombination between the additional marker and the exemplary SSR marker. Such determinations may utilize an improved method of orthogonal contrasts based on the method of Mather (1931), The Measurement of linkage in Heredity, Methuen & Co., London, followed by a test of maximum likelihood to determine a recombination frequency. Allard (1956) Hilgardia 24:235-78. If the value of the recombination frequency is less than or equal to 0.10 (i.e., 10%) in any cultivar, then the additional marker is considered equivalent to the particular reference marker for the purposes of use in the presently disclosed methods.

As used herein, the term “crossing” as used herein refers to the fertilization of female plants (or gametes) by male plants (or gametes). The term “gamete” refers to the haploid reproductive cell (egg or sperm) produced in plants by mitosis from a gametophyte and involved in sexual reproduction, during which two gametes of opposite sex fuse to form a diploid zygote. The term generally includes reference to a pollen (including the sperm cell) and an ovule (including the ovum). “Crossing” therefore generally refers to the fertilization of ovules of one individual with pollen from another individual, whereas “selfing” refers to the fertilization of ovules of an individual with pollen from the same individual. When referring to crossing in the context of achieving the introgression of a genomic region or segment., the skilled person will understand that in order to achieve the introgression of only a part of a chromosome of one plant into the chromosome of another plant, it is required that random portions of the genomes of both parental lines will be recombined during the cross due to the occurrence of crossing-over events in the production of the gametes in the parent lines. Therefore, the genomes of both parents must be combined in a single cell by a cross, where after the production of gametes from said cell and their fusion in fertilization will result in an introgression event.

The term “zygosity” means determining whether the source of nucleic acid appears heterozygous, homozygous, or hemizygous.

As used herein, the term “heterozygous” means a genetic condition existing when different alleles reside at corresponding loci on homologous chromosomes.

As used herein, the term “homozygous” means a genetic condition existing when identical alleles reside at corresponding loci on homologous chromosomes.

As used herein, the term “hemizygous” means having an allele of a gene at a given locus on one chromosome in the diploid state, for which there is no corresponding locus on the other chromosome of the pair.

As used herein, the term “introgression” refers to a genomic segment that has moved from one individual, species, variety or cultivar into the genome of another individual, species, variety or cultivar, by crossing those individuals, species, varieties or cultivars.

As used herein, the terms “introgressing”, “introgress” and “introgressed” refer to both a natural and artificial process whereby individual genes or entire traits are moved from one individual, species, variety or cultivar into the genome of another species, variety or cultivar, by crossing those species, varieties or cultivars. In plant breeding, the process usually involves selfing or backcrossing to the recurrent parent to provide for an increasingly homozygous plant having essentially the characteristics of the recurrent parent in addition to the introgressed gene or trait.

The term “backcross” refers to the process wherein the plant resulting from a cross between two parental lines is crossed with one of its parental lines, wherein the parental line used in the backcross is referred to as the recurrent parent. Repeated backcrossing results in the genome becoming more and more homozygous or inbred.

The term “selfing” refers to the process of self-fertilization wherein an individual is pollinated or fertilized with its own pollen.

Unless otherwise specifically explained, all technical and scientific terms used herein have the same meaning as commonly understood by those of ordinary skill in the art to which this disclosure belongs. Definitions of common terms in molecular biology can be found in, for example: Lewin, Genes V, Oxford University Press, 1994 (ISBN 0-19-854287-9); Kendrew et al. (eds.), The Encyclopedia of Molecular Biology, Blackwell Science Ltd., 1994 (ISBN 0-632-02182-9); and Meyers (ed.), Molecular Biology and Biotechnology: A Comprehensive Desk Reference, VCH Publishers, Inc., 1995 (ISBN 1-56081-569-8).

### Embodiments

A plant cell, callus, tissue or plant may be identified and isolated by selecting or screening the plant material for the N7/N16 homoeologous chromosomal reciprocal translocations in B. napus. Various assays can be employed to detect the polynucleotides that comprise the N7/N16 homoeologous chromosomal reciprocal translocations in B. napus. The following techniques are useful in a variety of situations, and in one embodiment, are useful in detecting the presence or absence of the N7/N16 homoeologous chromosomal reciprocal translocations in B. napus. For example, the presence of the molecule can be determined in a variety of ways, including using a primer or probe of the sequence in an amplification reaction. In an embodiment the disclosure relates method of detecting a plant comprising the N7/N16 homoeologous chromosomal reciprocal translocations in B. napus via an amplification reaction in which an amplified product or amplicon is generated. The detection of the amplicon is an indication of whether the plant contains the N7/N16 homoeologous chromosomal reciprocal translocations in B. napus.

Amplification of a nucleic acid sequence may be carried out by any suitable methodologies. See generally, Kwoh et al., Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad Sci. USA 86, 1173-1177 (1989)), self-sustained sequence replication (or “35R”) (see J. Guatelli et al., Proc. Natl. Acad. Sci. USA 87, 1874-1878 (1990)), the QP replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). Polymerase chain reaction is generally preferred.

The polynucleotides that comprise the N7/N16 homoeologous chromosomal reciprocal translocations in B. napus, or segments thereof, can be used as primers and/or probes for PCR amplification. In performing PCR amplification, a certain degree of mismatch can be tolerated between primer and template. Therefore, mutations, deletions, and insertions (especially additions of nucleotides to the 5′ end) of the exemplified primers fall within the scope of the subject disclosure. Mutations, insertions, and deletions can be produced in a given primer by methods known to an ordinarily skilled artisan.

As such, the chromosomal interval of: base pairs 17578019 to 17583020 of chromosome 7 from the DH12075 v1.1 genome is provided as SEQ ID NO:1; of base pairs 20106368 to 20111369 of chromosome 7 from the NS1822BC genome is provided as SEQ ID NO:2; of base pairs 13651438 to 13656439 of chromosome 16 from the DH12075 v1.1 genome is provided as SEQ ID NO:3; and, of base pairs 8089982 to 8094983 of chromosome 16 from the NS1822BC genome is provided as SEQ ID NO:4. In addition, probe sequences are provided herein: DHN16 probe sequences are provided as SEQ ID NO: 21-137; DHN7 probe sequences are provided as SEQ ID NO: 138-254; NSN16 probe sequences are provided as SEQ ID NO: 255-371; NSN7 probe sequences are provided as SEQ ID NO: 372-488. In addition, primer sequences are provided herein: DHN7 primer sequences are provided as SEQ ID NO: 489-2990, SEQ ID NO:5 SEQ ID NO:6, SEQ ID NO:13 and SEQ ID NO:15; DHN16 primer sequences are provided as SEQ ID NO: 2991-5492, SEQ ID NO:7, SEQ ID NO:8, SEQ ID NO:17 and SEQ ID NO:19; NSN7 primer sequences are provided as SEQ ID NO: 5493-8026, SEQ ID NO:9, SEQ ID NO: 10, SEQ ID NO:14 and SEQ ID NO:16; and, NSN16 primer sequences are provided as SEQ ID NO: 8027-10560, SEQ ID NO:11, SEQ ID NO:12, SEQ ID NO:18 and SEQ ID NO:20. These sequences provide exemplified compositions that can be used in an amplification reaction. Those with skill in the art could utilize the primer and probe sequences as disclosed to amplify a fragment of the genomic sequences and to detect the resulting amplicon.

In some embodiments an amplicon can be detected. In an aspect of this embodiment the amplicon is detected on an agarose gel via electrophoresis. Those with skill in the art appreciate the various types of agarose gel electrophoresis reagents and techniques that may be used. For instance pulsed field gel electrophoresis, pulsed orthogonal gel electrophoresis agarose multigel electrophoresis, and traditional agarose gel electrophoresis can be used with the subject disclosure. In further embodiments the size of the amplicon can be detected to determine whether the assay for the N7/N16 homoeologous chromosomal reciprocal translocations in B. napus. In an aspect the size of the amplicon can be about 50 base pairs, 100 base pairs, 150 base pairs, 200 base pairs, 250 base pairs, 300 base pairs, 350 base pairs, 400 base pairs, 450 base pairs, 500 base pairs, 550 base pairs, 600 base pairs, 650 base pairs, 700 base pairs, 750 base pairs, 800 base pairs, 850 base pairs, 900 base pairs, 950 base pairs, 1 kilobase pairs, 2 kilobase pairs, 3 kilobase pairs, 4 kilobase pairs, 5 kilobase pairs, 6 kilobase pairs, 7 kilobase pairs, 8 kilobase pairs, 9 kilobase pairs, 10 kilobase pairs, 11 kilobase pairs, 12 kilobase pairs, 13 kilobase pairs, 14 kilobase pairs, 15 kilobase pairs, 16 kilobase pairs, 17 kilobase pairs, 18 kilobase pairs, 19 kilobase pairs, or 20 kilobase pairs.

Following the completion of the PCR reaction and probe detection, a table and distribution graph may be generated using, for example, any suitable computer graphics software. Results obtained with wild-type, hemizygous and homozygous DNA of similar and/or known genotypic backgrounds may serve as positive or negative controls. In a segregating population, three clusters of data points may be obtained, allowing the visual determination of a sample result as likely belonging to one of the segregated clusters. Alternatively, data analysis computer software may be used to calculate the probability that a sample result belongs to each segregated cluster, with the most probable cluster serving as the sample designation. When a visual determination is made, the boundary of each cluster may be arbitrary, for example, when three clusters of data points are clearly visible.

Raw fluorescence intensity data may also be analyzed directly from a plate reader using a suitable analysis package, such as KLIMS (KBioscience laboratory information management system). A graph with the relative fluorescence units (RFU) of a fluorescence signal generated by a specific probe for a mutant allele plotted on one axis, and the RFU of a fluorescence signal generated by a specific probe for the wild-type allele plotted on the other axis may be generated. Zygosity determinations may then be made based on the cluster separation in a graphical display of the data.

Samples that do not contain the N7/N16 homoeologous chromosomal reciprocal translocation in a B. napus may only result in fluorescence readings of the wild-type PCR product. Samples containing hemizygous or homozygous mutant genomic DNA may result in RFU readings for the mutant-specific probe higher than that of a negative background control. If a sample yields no adequate results, the genomic DNA in the sample may not be of adequate quality and/or quantity, and a new DNA preparation and/or new PCR reaction should be performed. Preferably, a negative control sample containing no DNA sample shows very low detection of gene-specific probe(s). It is also preferable that known homozygous controls show only high detection of the mutant or wild-type DNA in the control, and that known hemizygous controls show both high detection of the mutant and wild-type DNA.

In an embodiment, the amplification reaction is quantified. In other embodiments, the amplification reaction is quantitated using a signature profile, in which the signature profile is selected from the group consisting of a melting temperature or a fluorescence signature profile. In some embodiments the amplification reaction is quantitated via the Delta Delta ct method. In other embodiments the amplification reaction is quantitated via the comparative cycle threshold method. In further embodiments the amplification reaction is quantitated via the standard curve method.

Standard Curve. Nucleic acids can be used to establish a standard curve. These methods are well known and include internal controls, double stranded DNA, a cDNA expressing a target gene, or an in vitro generated single stranded DNA. Methods may vary according to the nucleic acid chosen to serve as the standard to establish a standard curve.

Comparative Cycle Threshold. The comparative cycle threshold (Ct) method, also known as the 2^{-ΔΔct} method, is also used to quantify DNA levels. The Ct method compares a test reaction with a control or calibrator sample. The Ct values of both the control/calibrator sample and the test sample are normalized. In an embodiment of the invention, the Ct values were normalized to an arbitrary cutoff, 20-22. In another embodiment, the Ct values were normalized to within 1 Ct value of a negative control (a sample with no inhibition). This allows for the sensitivity of the assay and proper dynamic range.

Ct Method. The Ct method can also be described by the ΔΔCt formula; ΔΔCt=ΔCt_{test sample}−ΔCt_{reference sample}. The amplification efficiencies of the test sample and the reference sample must be about the same for the formula to operate. Amplification efficiencies can be determined by a comparison of the samples with template dilution. The amplification efficiency is about the same when a plot of cDNA dilution versus ΔCt approximates zero.

DNA may be isolated (for example, extracted, and purified) from plant tissue by methods known to those of skill in the art. Commercial kits for DNA isolation are available, for example, from Qiagen, Inc. In some embodiments, leaf discs from a particular plant are punched and transferred into collection tubes. The puncher may be cleaned after each sampling with 70% alcohol, rinsing in water, and drying. DNA extraction buffers may be prepared according to the manufacturer's recommendations. DNA may then be isolated using the kit according to the manufacturer's instructions. Finally, the concentration of the isolated DNA may be determined using, for example, a Quant-iT™ PicoGreen® Quantfication Kit (Invitrogen, Carlsbad, Calif) and a spectrophotometer, or by any other suitable technique.

In further embodiments, the compositions and methods of the subject disclosure may be used to design novel primer and probe sequences to the chromosomal interval of base pairs 17578019 to 17583020 of chromosome 7 from the DH12075 v1.1 genome is provided as SEQ ID NO:1. Those with skill in the art could utilize the disclosure to design primers of about 12 base pairs, 13 base pairs, 14 base pairs, 15 base pairs, 16 base pairs, 17 base pairs, 18 base pairs, 19 base pairs, 20 base pairs, 21 base pairs, 22 base pairs, 23 base pairs 24 base pairs, 25 base pairs, 26 base pairs, 27 base pairs, 28 base pairs, 29 base pairs, 30 base pairs or more that can bind this polynucleotide sequence (e.g. SEQ ID NO:1). The primers that can be designed to bind the sequence could either be directly complementary to the sequence or the reverse complement of the sequence. Likewise, those with skill in the art could utilize the disclosure to design probes of about 12 base pairs, 13 base pairs, 14 base pairs, 15 base pairs, 16 base pairs, 17 base pairs, 18 base pairs, 19 base pairs, 20 base pairs, or more that can bind this polynucleotide sequence (e.g. SEQ ID NO:1). The probes that can be designed to bind the sequence could either be directly complementary to the sequence or the reverse complement of the sequence. Exemplary DHN7 primer sequences are provided as SEQ ID NO: 489-2990, SEQ ID NO:5 SEQ ID NO:6, SEQ ID NO:13 and SEQ ID NO:15. Exemplary DHN7 probe sequences are provided as SEQ ID NO: 138-254. Those with skill in the art would be able to design and produce such compositions given that the chromosomal interval of base pairs 17578019 to 17583020 of chromosome 7 from the DH12075 v1.1 genome is provided as SEQ ID NO:1 is disclosed herein.

In further embodiments, the compositions and methods of the subject disclosure may be used to design novel primer and probe sequences to the chromosomal interval of base pairs 20106368 to 20111369 of chromosome 7 from the NS1822BC genome is provided as SEQ ID NO:2. Those with skill in the art could utilize the disclosure to design primers of about 12 base pairs, 13 base pairs, 14 base pairs, 15 base pairs, 16 base pairs, 17 base pairs, 18 base pairs, 19 base pairs, 20 base pairs, 21 base pairs, 22 base pairs, 23 base pairs 24 base pairs, 25 base pairs, 26 base pairs, 27 base pairs, 28 base pairs, 29 base pairs, 30 base pairs or more that can bind this polynucleotide sequence (e.g. SEQ ID NO:2). The primers that can be designed to bind the sequence could either be directly complementary to the sequence or the reverse complement of the sequence. Likewise, those with skill in the art could utilize the disclosure to design probes of about 12 base pairs, 13 base pairs, 14 base pairs, 15 base pairs, 16 base pairs, 17 base pairs, 18 base pairs, 19 base pairs, 20 base pairs, or more that can bind this polynucleotide sequence (e.g. SEQ ID NO:2). The probes that can be designed to bind the sequence could either be directly complementary to the sequence or the reverse complement of the sequence. Exemplary NSN7 primer sequences are provided as SEQ ID NO: 5493-8026, SEQ ID NO:9, SEQ ID NO:10, SEQ ID NO:14 and SEQ ID NO:16. Exemplary NSN7 probe sequences are provided as SEQ ID NO: 372-488. Those with skill in the art would be able to design and produce such compositions given that the chromosomal interval of base pairs 20106368 to 20111369 of chromosome 7 from the NS1822BC genome is provided as SEQ ID NO:2 is disclosed herein.

In further embodiments, the compositions and methods of the subject disclosure may be used to design novel primer and probe sequences to the chromosomal interval of base pairs 13651438 to 13656439 of chromosome 16 from the DH12075 v1.1 genome is provided as SEQ ID NO:3. Those with skill in the art could utilize the disclosure to design primers of about 12 base pairs, 13 base pairs, 14 base pairs, 15 base pairs, 16 base pairs, 17 base pairs, 18 base pairs, 19 base pairs, 20 base pairs, 21 base pairs, 22 base pairs, 23 base pairs 24 base pairs, 25 base pairs, 26 base pairs, 27 base pairs, 28 base pairs, 29 base pairs, 30 base pairs or more that can bind this polynucleotide sequence (e.g. SEQ ID NO:3). The primers that can be designed to bind the sequence could either be directly complementary to the sequence or the reverse complement of the sequence. Likewise, those with skill in the art could utilize the disclosure to design probes of about 12 base pairs, 13 base pairs, 14 base pairs, 15 base pairs, 16 base pairs, 17 base pairs, 18 base pairs, 19 base pairs, 20 base pairs, or more that can bind this polynucleotide sequence (e.g. SEQ ID NO:3). The probes that can be designed to bind the sequence could either be directly complementary to the sequence or the reverse complement of the sequence. Exemplary DHN16 primer sequences are provided as SEQ ID NO: 2991-5492, SEQ ID NO:7, SEQ ID NO:8, SEQ ID NO:17 and SEQ ID NO:19. Exemplary DHN16 probe sequences are provided as SEQ ID NO: 21-137. Those with skill in the art would be able to design and produce such compositions given that the chromosomal interval of base pairs 13651438 to 13656439 of chromosome 16 from the DH12075 v1.1 genome is provided as SEQ ID NO:3 is disclosed herein.

In further embodiments, the compositions and methods of the subject disclosure may be used to design novel primer and probe sequences to the chromosomal interval of base pairs base pairs 8089982 to 8094983 of chromosome 16 from the NS1822BC genome is provided as SEQ ID NO:4. Those with skill in the art could utilize the disclosure to design primers of about 12 base pairs, 13 base pairs, 14 base pairs, 15 base pairs, 16 base pairs, 17 base pairs, 18 base pairs, 19 base pairs, 20 base pairs, 21 base pairs, 22 base pairs, 23 base pairs 24 base pairs, 25 base pairs, 26 base pairs, 27 base pairs, 28 base pairs, 29 base pairs, 30 base pairs or more that can bind this polynucleotide sequence (e.g. SEQ ID NO:4). The primers that can be designed to bind the sequence could either be directly complementary to the sequence or the reverse complement of the sequence. Likewise, those with skill in the art could utilize the disclosure to design probes of about 12 base pairs, 13 base pairs, 14 base pairs, 15 base pairs, 16 base pairs, 17 base pairs, 18 base pairs, 19 base pairs, 20 base pairs, or more that can bind this polynucleotide sequence (e.g. SEQ ID NO:4). The probes that can be designed to bind the sequence could either be directly complementary to the sequence or the reverse complement of the sequence. Exemplary NSN16 primer sequences are provided as SEQ ID NO: 8027-10560, SEQ ID NO:11, SEQ ID NO:12, SEQ ID NO:18 and SEQ ID NO:20. Exemplary NSN16 probe sequences are provided as SEQ ID NO: 255-371. Those with skill in the art would be able to design and produce such a compositions given that the chromosomal interval of base pairs 8089982 to 8094983 of chromosome 16 from the NS1822BC genome is provided as SEQ ID NO:4 is disclosed herein.

In other embodiments, compositions and methods of the subject disclosure may be used in specific amplification assays such an end-point amplification assay to the N7/N16 homoeologous chromosomal reciprocal translocation. For example, an end-point PCR assay for testing the zygosity of B. napus containing the N7/N16 homoeologous chromosomal reciprocal translocations has been developed. In an embodiment, a method for determining the zygosity of the N7/N16 homoeologous chromosomal reciprocal translocations gene in a B. napus plant includes an amplification assay. Such an amplification or PCR assay can be quantitative and/or real-time and/or in a multiplex format. In an embodiment, a method employs TaqMan®-style probes (dual-labeled probes to fluoresce upon 5′→3′ exonuclease activity). In an embodiment, a method employs TaqMan®-style probes and oligonucleotides that selectively hybridize to the N7/N16 homoeologous chromosomal reciprocal translocation. In an embodiment, the N7/N16 homoeologous chromosomal reciprocal translocationprobes can be coupled to a detectable label at the 5′ end of the oligonucleotide. In an embodiment, the oligonucleotide can also be coupled to a quencher moiety at the 3′ end. An example of a quencher moiety for the N7/N16 homoeologous chromosomal reciprocal translocation is Black Hole Quencher™ (Biosearch Technologies, Novato, Calif). Suitable instrumentation will thereby detect the fluorescence produced from the cleavage of the oligonucleotide probe by the nuclease activity of the DNA polymerase during replication. Analysis software then determines the quantity of amplification product based upon the fluorescence data.

In an embodiment, an end-point method for the N7/N16 homoeologous chromosomal reciprocal translocation in a B. napus includes; a) performing a first PCR assay using a first probe, a first forward primer, and a first reverse primer on a polynucleotide sample from B. napus; b) quantifying the first probes; and c) confirming that the N7/N16 homoeologous chromosomal reciprocal translocation occurred within the B. napus genome. In an embodiment, the probes are detectably labeled. In an embodiment, the primers and probes are specific for the N7/N16 homoeologous chromosomal reciprocal translocation in a B. napus. In an embodiment probe sequences are provided herein: DHN16 probe sequences are provided as SEQ ID NO: 21-137; DHN7 probe sequences are provided as SEQ ID NO: 138-254; NSN16 probe sequences are provided as SEQ ID NO: 255-371; NSN7 probe sequences are provided as SEQ ID NO: 372-488. In an embodiment primer sequences are provided herein: DHN7 primer sequences are provided as SEQ ID NO: 489-2990, SEQ ID NO:5 SEQ ID NO:6, SEQ ID NO:13 and SEQ ID NO:15; DHN16 primer sequences are provided as SEQ ID NO: 2991-5492, SEQ ID NO:7, SEQ ID NO:8, SEQ ID NO:17 and SEQ ID NO:19; NSN7 primer sequences are provided as SEQ ID NO: 5493-8026, SEQ ID NO:9, SEQ ID NO:10, SEQ ID NO:14 and SEQ ID NO:16; and, NSN16 primer sequences are provided as SEQ ID NO: 8027-10560, SEQ ID NO:11, SEQ ID NO:12, SEQ ID NO:18 and SEQ ID NO:20. In an additional embodiment the N7/N16 homoeologous chromosomal reciprocal translocation is linked to the associated trait (native or transgenic) at a distance of about 1 megabase pairs, 950 kilobase pairs, 900 kilobase pairs, 850 kilobase pairs, 800 kilobase pairs, 750 kilobase pairs, 700 kilobase pairs, 650 kilobase pairs, 600 kilobase pairs, 550 kilobase pairs, 500 kilobase pairs, 450 kilobase pairs, 400 kilobase pairs, 350 kilobase pairs, 300 kilobase pairs, 250 kilobase pairs, 200 kilobase pairs, 150 kilobase pairs, 100 kilobase pairs, 90 kilobase pairs, 80 kilobase pairs, 70 kilobase pairs, 60 kilobase pairs, 50 kilobase pairs, 40 kilobase pairs, 30 kilobase pairs, 20 kilobase pairs, 10 kilobase pairs, 9 kilobase pairs, 8 kilobase pairs, 7 kilobase pairs, 6 kilobase pairs, 5 kilobase pairs, 4 kilobase pairs, 3 kilobase pairs, 2 kilobase pairs, 1 kilobase pairs, 750 base pairs, or 500 base pairs.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye. These fluorescent signals or fluorescent dyes provide exemplified compositions that can be used in an amplification reaction.

In other embodiments the quencher is selected from the group consisting of a Dabcyl quencher, a Tamra quencher, a Qxl quencher, an Iowa Black FQ quencher, an Iowa Black RQ quencher, an IR Dye QC-1 quencher, a Minor Groove Binding quencher, or a Blackhole quencher. These quenchers provide exemplified compositions that can be used in an amplification reaction.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed, such as YO-PRO-1@, SYTOX Green®, SYBR Green I®, SYTO11@, SYTO12@, SYTO13@, BOBO®, YOYO®, and TOTO®. in one embodiment, a second fluorescent DNA dye is SYTO13@used at less than 10 M, less than 4 μM, or less than 2.7 μM. These dyes provide exemplified compositions that can be used in an amplification reaction.

Target-specific primers and probes may be labeled, for example, with fluorescent dyes (e.g., FAM, VIC, and MGBNFQ), which may allow rapid quantification of a target-specific fluorescent signal. PCR products may be measured after a pre-determined number of cycles, for example, when the reaction is in the early exponential phase.

In some embodiments, a label comprises a fluorescent dye (e.g., a rhodamine dye (e.g., R6G, R110, TAMRA, ROX, etc.), a fluorescein dye (e.g., JOE, VIC, TET, HEX, FAM, etc.), a halofluorescein dye, a cyanine dye (e.g., CY3, CY3.5, CY5, CY5.5, etc.), a Bodipy® dye (e.g., FL, 530/550, TR, TMR, etc.), an Alexa Fluor® dye (e.g., 488, 532, 546, 568, 594, 555, 653, 647, 660, 680, etc.), a dichlororhodamine dye, an energy transfer dye (e.g., Bigdye®. v 1 dyes, Bigdye® v 2 dyes, Bigdye® v 3 dyes, etc.), Lucifer dyes (e.g., Lucifer yellow, etc.), Cascade Blue®, Oregon Green®, and the like. Fluorescent dyes can be distinguished and measured during amplification by their emitted excitation and/or emission spectra.

In an embodiment, a PCR assay method can include loading a PCR reaction mixture in a PCR assay tube, wherein the PCR reaction mixture comprises a polymerase with 5′ to 3′ nuclease activity, deoxynucleotides, a buffer, a first and a second forward primer, a first and a second reverse primer, a first and a second probe, and a polynucleotide sample, and wherein the first probe and the second probe comprise fluorescent dyes with distinguishable excitation/emission spectra; and performing an amplification step(s) under amplification conditions such that the 5′ to 3′ nuclease activity of the polymerase cleaves the first and second probes, thereby releasing fluorescent dyes comprising distinguishable excitation/emission spectra.

Compositions and methods of the subject disclosure may be used in specific amplification assays such as a zygosity assay. For example, an end-point PCR assay for testing the zygosity of B. napus containing the N7/N16 homoeologous chromosomal reciprocal translocations has been developed. This assay enables large scale and high throughput screening of B. napus germplasms with the N7/N16 homoeologous chromosomal reciprocal translocations. This assay will also increase the scale of using N7/N16 homoeologous chromosomal reciprocal translocations system for the development of B. napus lines.

In an embodiment, a method for determining zygosity of N7/N16 homoeologous chromosomal reciprocal translocation in a B. napus includes; a) performing a first PCR assay using a first probe, a first forward primer, and a first reverse primer on a polynucleotide sample from B. napus; b) performing a second PCR assay using a second probe, a second forward primer, and a second reverse primer on the polynucleotide sample from B. napus; c) quantifying the first and second probes; and d) comparing the quantified first and second probes to determine zygosity. In an embodiment, the probes are detectably labeled. In an embodiment, the primers and probes are specific for the N7/N16 homoeologous chromosomal reciprocal translocation in a B. napus. In an embodiment probe sequences are provided herein: DHN16 probe sequences are provided as SEQ ID NO: 21-137; DHN7 probe sequences are provided as SEQ ID NO: 138-254; NSN16 probe sequences are provided as SEQ ID NO: 255-371; NSN7 probe sequences are provided as SEQ ID NO: 372-488. In an embodiment primer sequences are provided herein: DHN7 primer sequences are provided as SEQ ID NO: 489-2990, SEQ ID NO:5 SEQ ID NO:6, SEQ ID NO:13 and SEQ ID NO:15; DHN16 primer sequences are provided as SEQ ID NO: 2991-5492, SEQ ID NO:7, SEQ ID NO:8, SEQ ID NO:17 and SEQ ID NO:19; NSN7 primer sequences are provided as SEQ ID NO: 5493-8026, SEQ ID NO:9, SEQ ID NO:10, SEQ ID NO:14 and SEQ ID NO:16; and, NSN16 primer sequences are provided as SEQ ID NO: 8027-10560, SEQ ID NO:11, SEQ ID NO:12, SEQ ID NO:18 and SEQ ID NO:20.

Compositions and methods of the subject disclosure may be used in specific amplification assays such an assay to detect trait introgression resulting from the N7/N16 homoeologous chromosomal reciprocal translocation. For example, an end-point PCR assay for testing the zygosity of B. napus containing the N7/N16 homoeologous chromosomal reciprocal translocations has been developed. This assay enables large scale and high throughput screening of B. napus germplasms with the N7/N16 homoeologous chromosomal reciprocal translocations. This assay will also increase the scale of using N7/N16 homoeologous chromosomal reciprocal translocations system for the development of B. napus lines. Through the detection of the N7/N16 homoeologous chromosomal reciprocal translocation any associated trait (native or transgenic) that is within about 1 megabase pairs, 950 kilobase pairs, 900 kilobase pairs, 850 kilobase pairs, 800 kilobase pairs, 750 kilobase pairs, 700 kilobase pairs, 650 kilobase pairs, 600 kilobase pairs, 550 kilobase pairs, 500 kilobase pairs, 450 kilobase pairs, 400 kilobase pairs, 350 kilobase pairs, 300 kilobase pairs, 250 kilobase pairs, 200 kilobase pairs, 150 kilobase pairs, 100 kilobase pairs, 90 kilobase pairs, 80 kilobase pairs, 70 kilobase pairs, 60 kilobase pairs, 50 kilobase pairs, 40 kilobase pairs, 30 kilobase pairs, 20 kilobase pairs, 10 kilobase pairs, 9 kilobase pairs, 8 kilobase pairs, 7 kilobase pairs, 6 kilobase pairs, 5 kilobase pairs, 4 kilobase pairs, 3 kilobase pairs, 2 kilobase pairs, 1 kilobase pairs, 750 base pairs, or 500 base pairs can be detected by the assay. Those with skill in the art, would appreciate that a linked trait will remain linked to the N7/N16 chromosomal regions after the resulting translocation, and that an assay to detect the N7/N16 chromosomal regions as they are translocated within the B. napus genome will indicate the presence of any linked trait. Especially, wherein the breakage point of the N7/N16 chromosomal translocation is identified as provided within this disclosure.

### Traits For Introgression

In some embodiments the assay to detect the N7/N16 homoeologous chromosomal reciprocal translocation in B. napus can be used to detect the introgression of traits (native and transgenic) via B. napus breeding programs.

Native traits of interest may be detected with the N7/N16 homoeologous chromosomal reciprocal translocation in B. napus assay of the subject disclosure. Exemplary native traits of interest that are suitable for use in the present disclosed constructs include, but are not limited to, coding sequences that confer (1) resistance to pests or disease, (2) tolerance to herbicides, (3) value added agronomic traits, such as; yield improvement, nitrogen use efficiency, water use efficiency, and nutritional quality, (4) binding of a protein to DNA in a site specific manner, (5) expression of small RNA, and (6) selectable markers. In accordance with one embodiment, the N7/N16 homoeologous chromosomal locus in B. napus is further stacked with at least one other native trait coding sequence encoding a gene product conferring insecticidal resistance, herbicide tolerance, small RNA expression, nitrogen use efficiency, water use efficiency, or nutritional quality.

Native traits of interest can include genes and traits f the seeds may be evaluated using techniques such as Near Infrared Reflectance Spectroscopy.

Transgenes of interest may be detected with the N7/N16 homoeologous chromosomal reciprocal translocation in B. napus assay of the subject disclosure. Exemplary transgenes of interest that are suitable for use in the present disclosed constructs include, but are not limited to, coding sequences that confer (1) resistance to pests or disease, (2) tolerance to herbicides, (3) value added agronomic traits, such as; yield improvement, nitrogen use efficiency, water use efficiency, and nutritional quality, (4) binding of a protein to DNA in a site specific manner, (5) expression of small RNA, and (6) selectable markers. In accordance with one embodiment, the N7/N16 homoeologous chromosomal locus in B. napus is further stacked with at least one other transgene/heterologous coding sequence encoding a selectable marker or a gene product conferring insecticidal resistance, herbicide tolerance, small RNA expression, nitrogen use efficiency, water use efficiency, or nutritional quality.

### 1. Insect Resistance

Various insect resistance genes can be further stacked with the N7/N16 homoeologous chromosomal locus in B. napus. The N7/N16 homoeologous chromosomal locus in B. napus can be operably linked with at least one other gene expression cassette containing an insect resistance gene. The operably linked sequences can then be incorporated into a chosen vector to allow for identification and selection of transformed plants (“transformants”). Exemplary insect resistance coding sequences are known in the art. As embodiments of insect resistance coding sequences that can be operably linked to the regulatory elements of the subject disclosure, the following traits are provided. Coding sequences that provide exemplary Lepidopteran insect resistance include: cry1A; cry1A.105; cry1Ab; cry1Ab (truncated); cry1Ab-Ac (fusion protein); cry1Ac (marketed as Widestrike®); cry1C; cry1F (marketed as Widestrike®); cry1Fa2; cry2Ab2; cry2Ae; cry9C; mocry1F; pinII (protease inhibitor protein); vip3A(a); and vip3Aa20. Coding sequences that provide exemplary Coleopteran insect resistance include: cry34Ab1 (marketed as Herculex®); cry35Ab1 (marketed as Herculex®); cry3A; cry3Bb1; dvsnf7; and mcry3A. Coding sequences that provide exemplary multi-insect resistance include ecry31.Ab. The above list of insect resistance genes is not meant to be limiting. Any insect resistance genes are encompassed by the present disclosure.

### 2. Herbicide Tolerance

Various herbicide tolerance genes can be can be further stacked with the N7/N16 homoeologous chromosomal locus in B. napus. The N7/N16 homoeologous chromosomal locus in B. napus can be operably linked with at least one other gene expression cassette containing a herbicide tolerance gene. The operably linked sequences can then be incorporated into a chosen vector to allow for identification and selection of transformed plants (“transformants”). Exemplary herbicide tolerance coding sequences are known in the art. As embodiments of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to the gene regulatory elements of the subject disclosure. In an embodiment, selectable marker genes include, but are not limited to genes encoding glyphosate resistance genes include: mutant EPSPS genes such as 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). These traits are currently marketed as Gly-Tol™, Optimum® GAT®, Agrisure® GT and Roundup Ready®. Resistance genes for glufosinate and/or bialaphos compounds include dsm-2, bar and pat genes. The bar and pat traits are currently marketed as LibertyLink®. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). These traits are marketed as Enlist® crop protection technology. Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csr1-2 genes, Sr-HrA genes, and surB genes. Some of the traits are marketed under the tradename Clearfield®. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyridinoxy or phenoxy proprionic acids and cyclohexones include the ACCase inhibitor-encoding genes (e.g., Acc1-S1, Acc1-S2 and Acc1-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), 1s+ genes (tolerance to triazine), and nitrilase genes (tolerance to benzonitrile). The above list of herbicide tolerance genes is not meant to be limiting. Any herbicide tolerance genes are encompassed by the present disclosure.

### 3. Agronomic Traits

Various agronomic trait genes can be further stacked with the N7/N16 homoeologous chromosomal locus in B. napus. The N7/N16 homoeologous chromosomal locus in B. napus can be operably linked with at least one other gene expression cassette containing an agronomic trait gene. The operably linked sequences can then be incorporated into a chosen vector to allow for identification and selection of transformed plants (“transformants”). Exemplary agronomic trait coding sequences are known in the art. As embodiments of agronomic trait coding sequences that can be operably linked to the regulatory elements of the subject disclosure, the following traits are provided. Delayed fruit softening as provided by the pg genes inhibit the production of polygalacturonase enzyme responsible for the breakdown of pectin molecules in the cell wall, and thus causes delayed softening of the fruit. Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes as provided by cspB genes maintain normal cellular functions under water stress conditions by preserving RNA stability and translation. Another example includes the EcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement is provided with the bbx32 gene that expresses a protein that interacts with one or more endogenous transcription factors to regulate the plant's day/night physiological processes. Ethanol production can be increase by expression of the amy797E genes that encode a thermostable alpha-amylase enzyme that enhances bioethanol production by increasing the thermostability of amylase used in degrading starch. Finally, modified amino acid compositions can result by the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

### 4. DNA Binding Proteins

Various DNA binding transgene/heterologous coding sequence genes/heterologous coding sequences can be can be further stacked with the N7/N16 homoeologous chromosomal locus in B. napus. The N7/N16 homoeologous chromosomal locus in B. napus can be operably linked with at least one other gene expression cassette containing a DNA binding gene. The operably linked sequences can then be incorporated into a chosen vector to allow for identification and selectable of transformed plants (“transformants”). Exemplary DNA binding protein coding sequences are known in the art. As embodiments of DNA binding protein coding sequences that can be operably linked to the regulatory elements of the subject disclosure, the following types of DNA binding proteins can include; Zinc Fingers, TALENS, CRISPRS, and meganucleases. The above list of DNA binding protein coding sequences is not meant to be limiting. Any DNA binding protein coding sequences is encompassed by the present disclosure.

### 5. Small RNA

Various small RNA sequences can be can be further stacked with N7/N16 homoeologous chromosomal locus in B. napus. The N7/N16 homoeologous chromosomal locus in B. napus can be operably linked with at least one other gene expression cassette containing a small RNA sequence. The operably linked sequences can then be incorporated into a chosen vector to allow for identification and selection of transformed plants (“transformants”). Exemplary small RNA traits are known in the art. As embodiments of small RNA coding sequences that can be operably linked to the regulatory elements of the subject disclosure, the following traits are provided. For example, delayed fruit ripening/senescence of the anti-efe small RNA delays ripening by suppressing the production of ethylene via silencing of the ACO gene that encodes an ethylene-forming enzyme. The altered lignin production of ccomt small RNA reduces content of guanacyl (G) lignin by inhibition of the endogenous S-adenosyl-L-methionine: trans-caffeoyl CoA 3-O-methyltransferase (CCOMT gene). Further, the Black Spot Bruise Tolerance in Solanum verrucosum can be reduced by the Ppo5 small RNA which triggers the degradation of Ppo5 transcripts to block black spot bruise development. Also included is the dvsnf7 small RNA that inhibits Western Corn Rootworm with dsRNA containing a 240 bp fragment of the Western Corn Rootworm Snf7 gene. Modified starch/carbohydrates can result from small RNA such as the pPhL small RNA (degrades PhL transcripts to limit the formation of reducing sugars through starch degradation) and pR1 small RNA (degrades R1 transcripts to limit the formation of reducing sugars through starch degradation). Additional, benefits such as reduced acrylamide resulting from the asn1 small RNA that triggers degradation of Asn1 to impair asparagine formation and reduce polyacrylamide. Finally, the non-browning phenotype of pgas ppo suppression small RNA results in suppressing PPO to produce apples with a non-browning phenotype. The above list of small RNAs is not meant to be limiting. Any small RNA encoding sequences are encompassed by the present disclosure.

### 6. Selectable Markers

Various selectable markers also described as reporter genes can be can be further stacked with the N7/N16 homoeologous chromosomal locus in B. napus. The N7/N16 homoeologous chromosomal locus in B. napus can be operably linked with at least one other gene expression cassette containing a reporter gene. The operably linked sequences can then be incorporated into a chosen vector to allow for identification and selectable of transformed plants (“transformants”). Many methods are available to confirm expression of selectable markers in transformed plants, including for example DNA sequencing and PCR (polymerase chain reaction), Southern blotting, RNA blotting, immunological methods for detection of a protein expressed from the vector. But, usually the reporter genes are observed through visual observation of proteins that when expressed produce a colored product. Exemplary reporter genes are known in the art and encode β-glucuronidase (GUS), luciferase, greenfluorescent protein (GFP), yellow fluorescent protein (YFP, Phi-YFP), red fluorescent protein (DsRFP, RFP, etc), β-galactosidase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for selection of transformed cells or tissues. Selectable marker genes include genes encoding antibiotic resistance, such as those encoding neomycin phosphotransferase II (NEO), spectinomycin/streptinomycin resistance (AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

In an embodiment, herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Acc1-S1, Acc1-S2 and Acc1-S3. In an embodiment, herbicides can inhibit photosynthesis, including triazine (psbA and 1s+ genes) or benzonitrile (nitrilase gene). Furthermore, such selectable markers can include positive selection markers such as phosphomannose isomerase (PMI) enzyme.

In an embodiment, selectable marker genes include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). An embodiment also includes selectable marker genes encoding resistance to: chloramphenicol; methotrexate; hygromycin; spectinomycin; bromoxynil; glyphosate; and phosphinothricin. The above list of selectable marker genes is not meant to be limiting. Any reporter or selectable marker gene are encompassed by the present disclosure.

In some embodiments the coding sequences are synthesized for optimal expression in a plant. For example, in an embodiment, a coding sequence of a gene has been modified by codon optimization to enhance expression in plants. An insecticidal resistance transgene, an herbicide tolerance transgene, a nitrogen use efficiency transgene, a water use efficiency transgene, a nutritional quality transgene, a DNA binding transgene, or a selectable marker transgene/heterologous coding sequence can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest.

In an embodiment, a coding sequence, gene, heterologous coding sequence or transgene/heterologous coding sequence is designed to be expressed in plants at a higher level resulting in higher transformation efficiency. Methods for plant optimization of genes are well known. Guidance regarding the optimization and production of synthetic DNA sequences can be found in, for example, WO2013016546, WO2011146524, WO1997013402, U.S. Pat. Nos. 6,166,302, and 5,380,831, herein incorporated by reference.

### Molecular Confirmation

Methods of confirming the N7/N16 homoeologous chromosomal reciprocal translocation in B. napus are known in the art. For example the detection of the N7/N16 homoeologous chromosomal reciprocal translocation in B. napus can be achieved, for example, by the polymerase chain reaction (PCR). The PCR detection is done by the use of two oligonucleotide primers flanking the polymorphic segment of the polymorphism followed by DNA amplification. This step involves repeated cycles of heat denaturation of the DNA followed by annealing of the primers to their complementary sequences at low temperatures, and extension of the annealed primers with DNA polymerase. Size separation of DNA fragments on agarose or polyacrylamide gels following amplification, comprises the major part of the methodology. Such selection and screening methodologies are well known to those skilled in the art. Molecular confirmation methods that can be used to identify transgenic plants are known to those with skill in the art. Several exemplary methods are further described below.

Molecular Beacons have been described for use in sequence detection. Briefly, a FRET oligonucleotide probe is designed that overlaps the flanking genomic and insert DNA junction. The unique structure of the FRET probe results in it containing a secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe(s) to the target sequence results in the removal of the probe secondary structure and spatial separation of the fluorescent and quenching moieties. A fluorescent signal indicates the presence of the flanking genomic/transgene insert sequence due to successful amplification and hybridization. Such a molecular beacon assay for detection of as an amplification reaction is an embodiment of the subject disclosure.

Hydrolysis probe assay, otherwise known as TAQMAN® (Life Technologies, Foster City, Calif.), is a method of detecting and quantifying the presence of a DNA sequence. Briefly, a FRET oligonucleotide probe is designed with one oligo within the transgene and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization. Such a hydrolysis probe assay for detection of as an amplification reaction is an embodiment of the subject disclosure.

KASPar® assays are a method of detecting and quantifying the presence of a DNA sequence. Briefly, the genomic DNA sample comprising the integrated gene expression cassette polynucleotide is screened using a polymerase chain reaction (PCR) based assay known as a KASPar® assay system. The KASPar® assay used in the practice of the subject disclosure can utilize a KASPar® PCR assay mixture which contains multiple primers. The primers used in the PCR assay mixture can comprise at least one forward primers and at least one reverse primer. The forward primer contains a sequence corresponding to a specific region of the DNA polynucleotide, and the reverse primer contains a sequence corresponding to a specific region of the genomic sequence. In addition, the primers used in the PCR assay mixture can comprise at least one forward primers and at least one reverse primer. For example, the KASPar® PCR assay mixture can use two forward primers corresponding to two different alleles and one reverse primer. One of the forward primers contains a sequence corresponding to specific region of the endogenous genomic sequence. The second forward primer contains a sequence corresponding to a specific region of the DNA polynucleotide. The reverse primer contains a sequence corresponding to a specific region of the genomic sequence. Such a KASPar® assay for detection of an amplification reaction is an embodiment of the subject disclosure.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed, such as YO-PRO-1®, SYTOX Green®, SYBR Green I®, SYTO11®, SYTO12®, SYTO13®, BOBO®, YOYO®, and TOTO®.

In further embodiments, Next Generation Sequencing (NGS) can be used for detection. As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction. Three NGS platforms are commercially available, the Genome Sequencer FLX™ from 454 Life Sciences/Roche, the Illumina Genome Analyser™ from Solexa and Applied Biosystems' SOLiD™ (acronym for: ‘Sequencing by Oligo Ligation and Detection’). In addition, there are two single molecule sequencing methods that are currently being developed. These include the true Single Molecule Sequencing (tSMS) from Helicos Bioscience™ and the Single Molecule Real Time™ sequencing (SMRT) from Pacific Biosciences.

The Genome Sequencher FLX™ which is marketed by 454 Life Sciences/Roche is a long read NGS, which uses emulsion PCR and pyrosequencing to generate sequencing reads. DNA fragments of 300-800 bp or libraries containing fragments of 3-20 kb can be used. The reactions can produce over a million reads of about 250 to 400 bases per run for a total yield of 250 to 400 megabases. This technology produces the longest reads but the total sequence output per run is low compared to other NGS technologies.

The Illumina Genome Analyser™ which is marketed by Solexa™ is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The Sequencing by Oligo Ligation and Detection (SOLiD) system marketed by Applied Biosystems™ is a short read technology. This NGS technology uses fragmented double stranded DNA that are up to 10 kb in length. The system uses sequencing by ligation of dye-labelled oligonucleotide primers and emulsion PCR to generate one billion short reads that result in a total sequence output of up to 30 gigabases per run.

The tSMS of Helicos Bioscience™ and SMRT of Pacific Biosciences™ apply a different approach which uses single DNA molecules for the sequence reactions. The tSMS Helicos™ system produces up to 800 million short reads that result in 21 gigabases per run. These reactions are completed using fluorescent dye-labelled virtual terminator nucleotides that is described as a ‘sequencing by synthesis’ approach.

The SMRT Next Generation Sequencing system marketed by Pacific Biosciences™ uses a real time sequencing by synthesis. This technology can produce reads of up to 1,000 bp in length as a result of not being limited by reversible terminators. Raw read throughput that is equivalent to one-fold coverage of a diploid human genome can be produced per day using this technology.

### Plant Compositions and Breeding

In other embodiments, the subject disclosure provides a cell, tissue, or plant comprising the N7/N16 homoeologous chromosomal reciprocal translocation in B. napus. In some embodiments, cell, tissue, or plant of B. napus in accordance with the present disclosure includes, but is not limited to, any variety or line of B. napus. Thus, any variety or line of B. napus be selected with the as provided subject disclosure. In embodiments, B. napus is in reference to the common name of rapeseed, oilseed rape, rape or canola. In some embodiments, the genetic background within a variety or line of B. napus may vary.

In a further embodiment, the subject disclosure provides a seed comprising the N7/N16 homoeologous chromosomal reciprocal translocation. In subsequent embodiments a seed from B. napus is provided. A B. napus seed may be composed of three structural parts: (1) the outer hull, which is a protective outer covering; and (2) the embryo (which also includes the cotyledons).

The subject disclosure also relates to one or more B. napus plant parts. B. napus plant parts include plant cells, plant protoplasts, plant cell tissue cultures from which plants can be regenerated, plant DNA, plant calli, plant clumps, and plant cells that are intact in plants or parts of plants, such as embryos, pollen, ovules, flowers, seeds, leaves, roots, root tips, anthers, and the like.

In subsequent embodiments, the subject disclosure relates to a B. napus cell comprising the N7/N16 homoeologous chromosomal reciprocal translocation. In other embodiments, the subject disclosure relates to a B. napus plant part comprising the N7/N16 homoeologous chromosomal reciprocal translocation. In further embodiments, the subject disclosure relates to a B. napus plant tissue comprising the N7/N16 homoeologous chromosomal reciprocal translocation. In additional embodiments, the subject disclosure relates to a B. napus plant comprising the N7/N16 homoeologous chromosomal reciprocal translocation. In some embodiments, the subject disclosure relates to a B. napus seed comprising the N7/N16 homoeologous chromosomal reciprocal translocation.

An embodiment of the subject disclosure provides a method for producing a B. napus seed comprising the N7/N16 homoeologous chromosomal reciprocal translocation, the method comprising the steps of: a) crossing a female parent plant with a male parent plant; b) harvesting a progeny seed from the cross of (a); c) planting the progeny seed; and, d) growing the progeny seed, wherein the progeny seed comprises the N7/N16 homoeologous chromosomal reciprocal translocation.

In additional embodiments, the subject disclosure relates to female and male parent plants that are B. napus plants. In further embodiments the male parent plant is isogenic to the female parent plant. In an aspect of the embodiment, the male parent plant is homozygous or heterozygous for the N7/N16 homoeologous chromosomal reciprocal translocation. In another aspect of the embodiment, the female parent plant is homozygous or heterozygous for the N7/N16 homoeologous chromosomal reciprocal translocation.

In yet another aspect of the subject disclosure, processes are provided for producing progeny plants, which processes generally comprise crossing a first parent plant with a second parent plant wherein at least one of the first parent plant or the second parent plant comprises the N7/N16 homoeologous chromosomal reciprocal translocation. These processes may be further exemplified as processes for producing progeny seed or plants, wherein a first plant is crossed with a second plant.

Any time the male plant is crossed with another, different inbred plant, a progeny or first generation (F₁) hybrid plant is produced. As such, a progeny or F₁ hybrid plant may be produced by crossing the first plant with any second inbred plant. Therefore, any progeny or F₁ hybrid plant or seed that comprises the N7/N16 homoeologous chromosomal reciprocal translocation which is produced with the first parent plant as a parent is an embodiment of the subject disclosure.

In embodiments of the present disclosure, the step of “crossing” the B. napus plant comprises planting, preferably in pollinating proximity, seeds of a first inbred B. napus plant and a second, distinct inbred B. napus plant. In other embodiments, the step of “crossing” the B. napus plant comprises planting, manually pollinating a first inbred B. napus plant with pollen to a second, distinct inbred B. napus plant.

In an embodiment, the B. napus plant that comprises the N7/N16 homoeologous chromosomal reciprocal translocation are treated with one or more agricultural chemicals as considered appropriate by the grower.

A further step comprises harvesting the seeds, near or at maturity, from the plant that received the pollen. In a particular embodiment, seed is harvested from the female parent plant, and when desired, the harvested seed can be grown to produce a progeny or first generation (F₁) hybrid plant.

Yet another step comprises drying and conditioning the seeds, including the treating, sizing (or grading) of seeds, and packaging for sale to growers for the production of oil and grain. As with inbred seed, it may be desirable to treat hybrid seeds with compositions that render the seeds and seedlings grown therefrom more hardy when exposed to adverse conditions. Mention should be made that resulting progeny or hybrid seed may be sold to growers for the production of oil and grain and not for breeding or seed production.

Still further, the subject disclosure provides a progeny plant produced by growing the harvested seeds produced on the B. napus plant that comprises the N7/N16 homoeologous chromosomal reciprocal translocation as well as grain produced by the progeny plant.

In an additional embodiment, the subject disclosure relates to a method for producing a progeny plant, the method further comprising the steps of: e) crossing the progeny plant, with another plant comprising a desired trait to produce F1 progeny plants; f) selecting F1 progeny plants that have the desired trait to produce selected F1 progeny plants; g) crossing the selected F1 progeny plants with the progeny B. napus plant that comprises the N7/N16 homoeologous chromosomal reciprocal translocation plant to produce backcross progeny plants; h) selecting for backcross progeny plants that have the desired trait; and, i) repeating steps (g) and (h) three or more times in succession to produce selected fourth or higher backcross progeny plants that comprise the desired trait.

Various breeding schemes may be used to produce progeny plants. In one method, generally referred to as the pedigree method, the parent may be crossed with another different plant such as a second inbred parent plant, which either itself exhibits one or more selected desirable characteristic(s) or imparts selected desirable characteristic(s) to a hybrid combination. If the two original parent plants do not provide all the desired characteristics, then other sources can be included in the breeding population. Progeny plants, that is, pure breeding, homozygous inbred lines, can also be used as starting materials for breeding or source populations from which to develop progeny plants.

Thereafter, resulting seed is harvested and resulting superior progeny plants are selected and selfed or sib-mated in succeeding generations, such as for about five to about seven or more generations, until a generation is produced that no longer segregates for substantially all factors for which the inbred parents differ, thereby providing a large number of distinct, pure-breeding inbred lines.

In another embodiment for generating progeny plants, generally referred to as backcrossing, one or more desired traits may be introduced into the parent by crossing the parent plants with another plant (referred to as the donor or non-recurrent parent) which carries the gene(s) encoding the particular trait(s) of interest to produce F₁ progeny plants. Both dominant and recessive alleles may be transferred by backcrossing. The donor plant may also be an inbred, but in the broadest sense can be a member of any plant variety or population cross-fertile with the recurrent parent. Next, F₁ progeny plants that have the desired trait are selected. Then, the selected progeny plants are crossed with the parent or restored fertile parent to produce backcross progeny plants. Thereafter, backcross progeny plants comprising the desired trait and the physiological and morphological characteristics of the parent are selected. This cycle is repeated for about one to about eight cycles, preferably for about three or more times in succession to produce selected higher backcross progeny plants that comprise the desired trait and all of the physiological and morphological characteristics of the parent when grown in the same environmental conditions. Exemplary desired trait(s) include insect resistance, enhanced nutritional quality, herbicide resistance, yield stability, yield enhancement and resistance to bacterial, fungal and viral disease. One of ordinary skill in the art of plant breeding would appreciate that a breeder uses various methods to help determine which plants should be selected from the segregating populations and ultimately which inbred lines will be used to develop hybrids for commercialization. In addition to the knowledge of the germplasm and other skills the breeder uses, a part of the selection process is dependent on experimental design coupled with the use of statistical analysis. Experimental design and statistical analysis are used to help determine which plants, which family of plants, and finally which inbred lines and hybrid combinations are significantly better or different for one or more traits of interest. Experimental design methods are used to assess error so that differences between two inbred lines or two hybrid lines can be more accurately determined. Statistical analysis includes the calculation of mean values, determination of the statistical significance of the sources of variation, and the calculation of the appropriate variance components. Either a five or a one percent significance level is customarily used to determine whether a difference that occurs for a given trait is real or due to the environment or experimental error. One of ordinary skill in the art of plant breeding would know how to evaluate the traits of two plant varieties to determine if there is no significant difference between the two traits expressed by those varieties. For example, see Fehr, Walt, Principles of Cultivar Development, p. 261-286 (1987) which is incorporated herein by reference. Mean trait values may be used to determine whether trait differences are significant, and preferably the traits are measured on plants grown under the same environmental conditions.

This method results in the generation of progeny inbred plants with substantially all of the desired morphological and physiological characteristics of the recurrent parent and the particular transferred trait(s) of interest. Because such progeny inbred plants are heterozygous for loci controlling the transferred trait(s) of interest, the last backcross generation would subsequently be selfed to provide pure breeding progeny for the transferred trait(s).

In other embodiments for generating progeny plants the subject disclosure relates to the production of doubled haploid plants produced from B. napus microspores, wherein the doubled haploid plants comprise the N7/N16 homoeologous chromosomal reciprocal translocation. Those with skill in the art would appreciate that a microspore derived embryo may be produced from microspores that are obtained from the anthers of B. napus. These microspores—formed from pollen mother cells that undergo meiosis—are generated into embryos and later into mature plant by maintenance on the appropriate culture media followed by transfer to soil. The microspores are haploid cells. Those with skill in the art would understand that in some instances a haploid cell may spontaneously give rise to a diploid cell or cells following mitosis. Likewise, those with skill in the art would understand that a chromosome-doubling agent may be applied to increase the ploidy of a haploid cell. In aspects of such an embodiment the haploid cell becomes a diploid cell. Such a diploid cell may give rise to a B. napus plant comprising the N7/N16 homoeologous chromosomal reciprocal translocation. The methods of the present disclosure do not depend on a particular genetic mechanism of chromosome doubling. Typically, induced chromosome doubling of the invention involves administering an effective amount of a chromosome-doubling agent to a cell, preferably a haploid cell. Any agent that is known to increase the ploidy of cells may be employed in the methods of the subject disclosure. Exemplary, and non-limiting, chromosome doubling agents include, but are not limited to, trifluralin, colchicine, oryzalin, amiprophosmethyl and pronamide. Depending on the desired outcome, a chromosome-doubling agent may be administered to a tissue, or a cell thereof, but, is usually the doubling agent is applied directly to microspores via addition to the culture medium. In certain aspects of this embodiment, an effective amount of a chromosome-doubling agent is administered after obtaining the haploid embryo. In other aspects of this embodiment, an effective amount of a chromosome-doubling agent is administered at the microspore stage of B. napus development. Accordingly, the subject disclosure provides methods and compositions for developing progeny plants comprising the N7/N16 homoeologous chromosomal reciprocal translocation from B. napus microspores.

In further embodiments the subject disclosure relates to the determination of recombinant genetic frequencies in B. napus plants comprising the N7/N16 homoeologous chromosomal reciprocal translocation. In certain aspects, doubled haploid B. napus plants comprising the N7/N16 homoeologous chromosomal reciprocal translocation produced from microspores are used to determine the recombinant genetic frequencies. In other aspects, progeny B. napus plants comprising the N7/N16 homoeologous chromosomal reciprocal translocation are used to determine the recombinant genetic frequencies. In an aspect of this embodiment, the assays disclosed for the N7/N16 homoeologous chromosomal reciprocal translocation can be performed to identify linkage disequilibrium within the disclosed genomic sequences; for example, SEQ ID NO:1, SEQ ID NO:2, SEQ ID NO:3 and/or SEQ ID NO:4. The linkage of the polynucleotides from these genomic sequences (e.g., SEQ ID NO:1, SEQ ID NO:2, SEQ ID NO:3, and/or SEQ ID NO:4) can be assayed using the methodologies and compositions as provided within this disclosure to determine the relative genetic distance to the N7/N16 homoeologous chromosomal reciprocal translocation. In an aspect of this embodiment, the assays disclosed for the N7/N16 homoeologous chromosomal reciprocal translocation can be performed to identify linkage disequilibrium within any B. napus genomic sequence on chromosome 7 or chromosome 16. The linkage of the polynucleotides from any B. napus genomic sequence on chromosome 7 or chromosome 16 can be assayed using the methodologies and compositions as provided within this disclosure to determine the relative genetic distance to the N7/N16 homoeologous chromosomal reciprocal translocation. In some aspects the linkage between the polynucleotides assayed from the genomic sequences can be linked, tightly linked, or extremely tightly linked to the N7/N16 homoeologous chromosomal reciprocal translocation. Those with skill in the art would appreciate that linkage of any polynucleotide sequence from the genomic sequences as disclosed herein (for example, SEQ ID NO:1, SEQ ID NO:2, SEQ ID NO:3, and/or SEQ ID NO:4) to the breakage point of the N7/N16 homoeologous chromosomal reciprocal translocation may refer to the phenomenon in which the polynucleotides show a measurable probability of being passed on with the N7/N16 homoeologous chromosomal reciprocal translocation in the next generation. Likewise, those with skill in the art would appreciate that linkage of any polynucleotide sequence from within any B. napus genomic sequence on chromosome 7 or chromosome 16 to the breakage point of the N7/N16 homoeologous chromosomal reciprocal translocation may refer to the phenomenon in which the polynucleotides show a measurable probability of being passed on with the N7/N16 homoeologous chromosomal reciprocal translocation in the next generation. Thus, those with skill in the art would appreciate that the linkage of one polynucleotide sequence to another polynucleotide sequence may be measured and/or expressed as a recombination frequency. When the presence of a gene contributes to a phenotype in an individual, any polynucleotide sequences that are linked to the gene may be said to be linked to the phenotype. Accordingly, in aspects of this disclosure when the presence of the N7/N16 homoeologous chromosomal reciprocal translocation contributes to a phenotype in an individual, the polynucleotide sequences of the N7/N16 homoeologous chromosomal reciprocal translocation as provided in this subject disclosure that are linked to the gene may be said to be linked to the phenotype.

In a subsequent embodiment, the disclosure related to introducing a desired trait into B. napus plants comprising the N7/N16 homoeologous chromosomal reciprocal translocation plant. In an aspect of the embodiment, the desired trait is selected from the group consisting of a native trait or a transgenic trait that includes an insecticidal resistance trait, herbicide tolerant trait, disease resistance trait, yield increase trait, nutritional quality trait, agronomic increase trait, and combinations thereof.

The Introgression of a desirable trait in plants may be facilitated by repeated backcrossing. Described herein are methods for producing a B. napus plant with N7/N16 homoeologous chromosomal reciprocal translocation, through conventional plant breeding involving sexual reproduction. Methods may comprise crossing a first parent plant that comprises in its genome at least one copy of a trait to a second parent plant, so as to produce F₁ progeny. The first plant can be any plant or variety of B. napus. The second parent plant can be any plant that is capable of producing viable progeny plants (i.e., seeds) when crossed with the first plant. The first and second parent plants may be of the same B. napus line or variety. The methods may further involve selfing the F₁ progeny to produce F₂ progeny. Methods may further involve one or more generations of backcrossing the F1 or F₂ progeny plants to a plant of the same line or genotype as either the first or second parent plant. Alternatively, the F1 progeny of the first cross, or any subsequent cross, can be crossed to a third plant that is of a different line or genotype than either the first or second plant.

In some embodiments, progeny plants are subjected to a genotype and/or zygosity determination, as outlined in the disclosure. Once progeny plants have been genotyped, and/or their zygosity determined, the skilled artisan may select those progeny plants that have a desired genetic composition. Such selected progeny plants may be used in further crosses, selfing, or cultivation. Methods of introgression of a trait that are directed according to methods of the disclosure reduce or eliminate the cultivation and/or reproduction of plants that do not have a desired genetic composition, and thereby provide desirable reliability and predictability (through expected Mendelian patterns of inheritance).

Backcrossing may be accelerated by the use of the N7/N16 homoeologous chromosomal reciprocal translocation assays of the pending application to identify plants with the greatest genetic complement from the recurrent parent.

Direct selection may be applied where a single locus acts as a dominant trait, such as the herbicide resistance trait. For this selection process, the progeny of the initial cross are sprayed with the herbicide before the backcrossing. The spraying eliminates any plants which do not have the desired herbicide resistance characteristic, and only those plants which have the herbicide resistance gene are used in the subsequent backcross. In the instance where the characteristic being transferred is a recessive allele, it may be necessary to introduce a test of the progeny to determine if the desired characteristic has been successfully transferred. The process of selection, whether direct or indirect, is then repeated for all additional backcross generations.

It should be appreciated by those having ordinary skill in the art that backcrossing can be combined with pedigree breeding as where the parent is crossed with another plant, the resultant progeny are crossed back to the parent or restored fertile parent and thereafter, the resulting progeny of this single backcross are subsequently inbred to develop new inbred lines. This combination of backcrossing and pedigree breeding is useful as when recovery of fewer than all of the parent characteristics than would be obtained by a conventional backcross are desired.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

Embodiments of the subject disclosure are further exemplified in the following Examples. It should be understood that these Examples are given by way of illustration only. From the above embodiments and the following Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The following is provided by way of illustration and not intended to limit the scope of the invention.

### EXAMPLES

### Example 1: Discovery and Validation of Chromosomal N7/N16 Reciprocal Translocation Break Points in B. napus

To discover the location of the N7/N16 chromosomal break point in B. napus, an end-to-end chromosome assembly based on single molecule long read sequencing for B. napus line NS1822BC was developed. The resulting reference genetic map of line NS1822BC revealed the presence of an N7/N16 homoeologous chromosomal reciprocal translocation. Using a global alignment of the NS1822BC genome with that of publicly available B. napus line DH12075 genome (which does not contain the N7/N6 homoeologous chromosomal reciprocal translocation), the location and sequences of the two break-points on N7 and N16 chromosomes were identified for the first time. As provided herein, SEQ ID N04: provides the sequence of the N7/N16 homoeologous chromosomal reciprocal translocation of chromosome 16 as compared to the wildtype sequence of chromosome 16 of SEQ ID NO:3. As provided herein, SEQ ID NO:2: provides the sequence of the N7/N16 homoeologous chromosomal reciprocal translocation of chromosome 7 as compared to the wildtype sequence of chromosome 7 of SEQ ID NO:1. This analysis resulted in the precise estimation of the genetic and physical length of the N7/N16 homoeologous chromosomal reciprocal translocation (Table 1). Furthermore, it was discovered that nearly one-third of the bottom end of the N7 chromosome (a 37 cM segment starting from 65.8 cM to the end of the chromosome at 102.8 cM in genetic space and an ~7.8 Mb physical segment starting from 17580520 bp to 25458507 on DH12075 genome) was involved in the homoeotic exchange with N16 chromosome. Similarly, an ~9.8 cM genetic segment (0-9.8 cM) at the top of the N16 chromosome (~8.1 Mb physical segment starting from 0-13653938 bp on DH12075 genome) was involved in the homoeotic exchange with N7 chromosome. These exchanged segments were positioned in an inverted orientation in the individuals containing N7/N16 HRT.

Next, the N7/N16 homoeologous chromosomal reciprocal translocation was assessed and characterized in elite germplasm lines from North America (NA), Australia (AU), and Europe (EU). From this analysis, it was determined that the N7/N16 homoeologous chromosomal reciprocal translocation is fixed in NA germplasm lines, nearly absent in EU germplasm lines, and segregates in AU germplasm lines (Table 2).

TABLE 1
Physical co-ordinates of the N7/N16 homoeologous
chromosomal reciprocal translocation break-points

| | | Break point position | Break point position |
|---|---|---|---|
| Genome | Chromosome | left in bp | right in bp |
| DH12075 v1.1 | N7 | 17580519 | 17580520 |
| DH12075 v1.1 | N16 | 13653938 | 13653939 |
| NS1822BC | N7 | 20108868 | 20108869 |
| NS1822BC | N16 | 8092482 | 8092483 |

TABLE 2
Germplasm characterization

| | Total | Lines with | Lines without |
|---|---|---|---|
| Breeding Geography | lines | Translocation | Translocation |
| Australian Spring type | 145 | 47 | 98 |
| European Winter type | 255 | 4 | 251 |
| North American Spring | 434 | 400 | 34 |
| type | | | |
| Total | 834 | 451 | 383 |

### Example 2: Detection of N7/N16 Homoeologous Chromosomal Reciprocal Translocation Using Agarose-Based PCR Assays

The genome sequence of the public DH12075 line that does not contain the N7/N16 translocation was used to develop two “wild type PCR assays” to detect the absence of the reciprocal chromosomal exchanges at both the N7 and N16 chromosomal sites respectively. The assays were designed using primers (Primer3 software: available on the world wide web at bioprod.phibred.com/primer3/cgi-bin/primer3_www.cgi) that flank the site of break-point sequences (Table 3, and Table 4). PCR amplification products from these assays when run on an (1%) agarose gel produced a band of 2,366 bp for N7 and 1,963 bp for N16, diagnostic for the wild type configuration or absence of the N7/N16 translocation.

The genome sequence of the proprietary N1822BC line that does contain the N7/N16 translocation was used to develop two PCR assays each on N7 and N16 that detect the reciprocal translocation. These assays were developed using primers that flank the chromosome exchange points on N7 and N16 chromosome sequences (Table 3, and Table 4). When run on the agarose gels these assays produce products of 1,828 bp for N7 and 1,893 bp for N16 that will detect the presence of N7 and N16 reciprocal recombination.

TABLE 3
Sequence IDs and physical positions used in the Detection of N7/N16 homoeologous
chromosomal reciprocal translocation using agarose-based PCR assays

| SEQ ID | Sequence | | | Physical | Physical |
|---|---|---|---|---|---|
| NO: | description | Genome | Chromosome | location (start) | location (end) |
| SEQ ID | N7 HRT | DH12075 | N7 | 17578019 | 17583020 |
| NO: 1 | Seq 1 | v1.1 | | | |
| SEQ ID | N7 HRT | NS1822BC | N7 | 20106368 | 20111369 |
| NO: 2 | Seq 2 | | | | |
| SEQ ID | N16 HRT | DH12075 | N16 | 13651438 | 13656439 |
| NO: 3 | Seq 1 | v1.1 | | | |
| SEQ ID | N16 HRT | NS1822BC | N16 | 8089982 | 8094983 |
| NO: 4 | Seq 2 | | | | |

TABLE 4
Primer IDs, physical positions, and sequences used in the
Detection of N7/N16 homoeologous chromosomal reciprocal
translocation using agarose-based PCR assays

| | | Reference | | |
|---|---|---|---|---|
| SEQ ID | | physical | Sequence ID | |
| NO | Primer ID | position | physical position | Sequence |
| SEQ ID | DH N7 F | DH12075 | N7 HRT Seq 1: | TTTAACAAAGTAAGGT |
| NO: 1 | | N7: 17580813 | 2501 | GTTTCTTGTG |
| | | | | SEQ ID NO: 5 |
| SEQ ID | DH N7 R | DH12075 | N7 HRT Seq 1: | TTAAGCCAACATGTGC |
| NO: 2 | | N7: 17578511 | 174 | AGGA |
| | | | | SEQ ID NO: 6 |
| SEQ ID | DH N16 F | DH12075 | N16 HRT Seq 1: | AGCGACTTTTCACAAA |
| NO: 3 | | N16: 13653931 | 2132 | GTAGGG |
| | | | | SEQ ID NO: 7 |
| SEQ ID | DH N16 R | DH12075 | N16 HRT Seq 1: | TGATGGTTAAAACCCA |
| NO: 4 | | N16: 13655875 | 4094 | CTGC |
| | | | | SEQ ID NO: 8 |
| SEQ ID | NS N7 F | NS1822BC | N7 HRT Seq 2: | CAACGACTTTTCACAA |
| NO: 5 | | N7: 20108854 | 2509 | AGTAGGG |
| | | | | SEQ ID NO: 9 |
| SEQ ID | NS N7 R | NS1822BC | N7 HRT Seq 2: | GCCTCAGTTATTATGT |
| NO: 6 | | N7: 20107032 | 665 | TTGTGATGG |
| | | | | SEQ ID NO: 10 |
| SEQ ID | NS N16 F | NS1822BC | N16 HRT Seq 2: | GTGATGGTTAAAACCC |
| NO: 7 | | N16: 8094055 | 4074 | ACTGC |
| | | | | SEQ ID NO: 11 |
| SEQ ID | NS N16 R | NS1822BC | N16 HRT Seq 2: | TTTAACAAAGTAAGGT |
| NO: 8 | | N16: 8092164 | 2183 | GTTTCTTGTG |
| | | | | SEQ ID NO: 12 |

The lines NS1882BC (confirmed to have the translocation) and DH12075 (confirmed to not contain the translocation) were used as positive and negative controls when testing the effectiveness of the PCR assay to detect the presence or absence of translocation. PCR reactions were set up in 20 ul volume containing 2 ul of DNA (~8 ng), 4 ul of 100 mM primers (2 ul forward 2 ul reverse), 10 ul Phusion GC Master Mix™ (Fisher scientific) and 4 ul of water. PCR conditions were as follows: 98° C. for 30 secs, followed by 35 cycles of 98° C. for 10 secs, 61° C. for 30 secs, and 72° C. for 120 secs, with a final extension of 72° C. for 7 mins and a final infinite hold at 4° C. PCR products were separated on a 1% agarose gel (SeaKem GTG Agarose™ Lonza) containing Sybr Safe (Sybr Safe DNA gel Stain™, Unity) in 1×TAE buffer (Omnipur TAE buffer, Thermo Fisher Scientific) for 1 h and visualized and photographed using an Alpha Imager utilizing a bottom illumination plate set to emit light at ~530 nm. Gel images were scored with a score of one (1) representing presence and a score of zero (0) representing absence of the reciprocal translocation.

### Example 3: Detection of N7/N16 Homoeologous Chromosomal Reciprocal Translocation Using High-Throughput Competitive Allele-Specific PCR Genotyping (KASPR™) Assays

High-throughput markers greatly facilitate the large-scale germplasm screening and breeding. Co-dominant assays that allow detection of translocated lines from non-translocated for N7/N16 homoeologous chromosomal reciprocal translocation were designed using the breakpoint sites at both N7 and N16 sequences. The KASPAR™ genotyping system is comprised of two components (1) the SNP-specific assay (a combination of three unlabelled primers), and (2) the universal Reaction Mix, which contains all other required components including the universal fluorescent reporting system and a specially-developed Taq polymerase. The four primers, allele-specific 1 (A1), allele-specific 2 (A2), and two common (C1 and C2), or reverse, (Table 5) were designed using the assay design algorithm of the workflow manager, Kraken™ (LGC Genomics, Hoddesdon, Hertfordshire, UK).

TABLE 5
Primer identification and physical positions, used in the
detection of N7/N16 homoeologous chromosomal reciprocal
translocation using KASP ™ assays:

| SEQ | | | Reference | | |
|---|---|---|---|---|---|
| ID | | | physical | Sequence ID | |
| NO: | Primer | Primer ID | position | physical position | Sequence |
| SEQ | N7 A1 | N7_S3 F1 | DH12075 | N7 HRT | GTCCGTTTGCTTTAAT |
| ID | | | N7: 17580752 | Seq 1: | AAGCCCAA |
| NO: 13 | | | | 2415 | SEQ ID NO: 13 |
| SEQ | N7 A2 | N7_S3 F2 | NS1822BC | N7 HRT | TCCCGTGTGCTTTAAT |
| ID | | | N7: 20108782 | Seq 2: | AAGCCCAG |
| NO: 14 | | | | 2416 | SEQ ID NO: 14 |
| SEQ | N7 C1 | Cr5 DH | DH12075 | N7 HRT | AGATCAGCGACTTTTC |
| ID | | | N7: 17580893 | Seq 1: | ACAAA |
| NO: 15 | | | | 2875 | SEQ ID NO: 15 |
| SEQ | N7 C2 | Cr5 NS | NS1822BC | N7 HRT | CCAGTTTTCAGAGGG |
| ID | | | N7: 20108860 | Seq 2: | AGTTTT |
| NO: 16 | | | | 2556 | SEQ ID NO: 16 |
| SEQ | N16 A1 | DH16_PA F | DH12075 | N16 HRT | ACCTCAACCTCAACCC |
| ID | | | N16: 13654237 | Seq 1: | CAATC |
| NO: 17 | | | | 2437 | SEQ ID NO: 17 |
| SEQ | N16 A2 | N16_NS_F2 | NS1822BC | N16 HRT | GGAGGAGGAGGAGGA |
| ID | | | N16: 8092483 | Seq 2: | GTCGTT |
| NO: 18 | | | | 2501 | SEQ ID NO: 18 |
| SEQ | N16 C1 | DH16_PA_ | DH12075 | N16 HRT | GGGATTGGATTACGG |
| ID | | Cr2 | N16: 13654263 | Seq 1: | TTTTCGGGTT |
| NO: 19 | | | | 2487 | SEQ ID NO: 19 |
| SEQ | N16 C2 | NS16_PA_CR1 | NS1822BC | N16 HRT | ATTTTCCACATGGGTG |
| ID | | | N16: 8092428 | Seq 2: | AACCACAA |
| NO: 20 | | | | 2470 | SEQ ID NO: 20 |

Primers were designed using the LGC Genomics assay design software. As such, 1.5 ul of ~4 ng/ul of DNA was used in the assay mix (~6 ng), and 12 ul of 100 mM of each forward primer and 30 ul of 100 mM of each of the common reverse primers and 16 ul of water was combined to make each assay. Next, 13.6 ul of the assay was combined with 1000 ul of KASP Master Mix™ (LGC Genomics, Hoddesdon, Hertfordshire, UK). A Meridian™ (LGC Genomics, Hoddesdon, Hertfordshire, UK) liquid handler dispensed 1.3 ul of the mix onto a 1536 plate containing ~6 ng of dried DNA. The plate was sealed with a Phusion™ laser sealer (LGC Genomics, Hoddesdon, Hertfordshire, UK) and thermocycled using a LGC Genomics™ Hoddesdon, Hertfordshire, UK hydrocycler with the following conditions: 95° C. for 15 min, 10 cycles of 95° C. for 20 sec, 61° C. stepped down to 55° C. for 1 min, 29 cycles of 95° C. for 20 sec, and 55° C. for 1 min. The excitation at wavelengths 485 (FAM) and 520 (VIC) was measured with a Pherastar™ plate reader (BMG Labtech, Offenburg, Germany). The values were normalized against ROX and plotted and scored on scatterplots utilizing the KRAKEN software (LGC Genomics, Hoddesdon, Hertfordshire, UK).

### Example 4: Detection of N7/N16 Homoeologous Chromosomal Reciprocal Translocation Using an Endpoint Assay Development

Generally, the endpoint genotyping system is comprised of a forward and reverse primer and two fluoresce labeled probes. The oligonucleotide primers amplify a specific region of the wildtype N7 or N16 chromosome, and the translocated N7 or N16 chromosome. The oligonucleotide probe binds to the amplicon between the two primers and are labeled with the VIC or FAM fluorescent reporter dye, at the 5′ end and MGBNFQ (minor grove binding non-fluorescent quencher) as a quencher at the 3′ end.

An exhaustive listing of endpoint genotyping systems (e.g., TAQMAN™, KASPAR™ or other known assays) that rely on amplifiable primers (SEQ ID NO:489-10560) and probes (SEQ ID NO:21-488) that spanned the N7/N16 translocation break-point site were designed. As provided herein the wildtype chromosome 7 can be assayed with SEQ ID NO:489-2990 and probed with SEQ ID NO:138-254. The assay for wildtype chromosome 7 utilizes any of SEQ ID NO:489-1739 as a forward or 5′ primer, in combination with any of SEQ ID NO:1740-2990 as a reverse or 3′ primer, and in combination any of SEQ ID NO:138-254 as a probe. As provided herein the wildtype chromosome 16 can be assayed with SEQ ID NO:2991-5492 and probed with SEQ ID NO:21-137. The assay for wildtype chromosome 16 utilizes any of SEQ ID NO:2991-4241 as a forward or 5′ primer, in combination with any of SEQ ID NO:4242-5492 as a reverse or 3′ primer, and in combination any of SEQ ID NO: 21-137 as a probe. As provided herein the homoeologous chromosomal reciprocal translocation of chromosome 7 can be assayed with SEQ ID NO:5493-8026 and probed with SEQ ID NO:372-488. The assay for the homoeologous chromosomal reciprocal translocation of chromosome 7 utilizes any of SEQ ID NO:5493-6759 as a forward or 5′ primer, in combination with any of SEQ ID NO:6760-8026 as a reverse or 3′ primer, and in combination any of SEQ ID NO:372-488 as a probe. As provided herein the homoeologous chromosomal reciprocal translocation of chromosome 16 can be assayed with SEQ ID NO:8027-10560 and probed with SEQ ID NO:255-371. The assay for the homoeologous chromosomal reciprocal translocation of chromosome 16 utilizes any of SEQ ID NO:8027-9294 as a forward or 5′ primer, in combination with any of SEQ ID NO:9295-10560 as a reverse or 3′ primer, and in combination any of SEQ ID NO:255-371as a probe. Those with skill in the art can utilize such compositions to devise an assay for the detection of N7/N16 homoeologous chromosomal reciprocal translocation in B. napus.

These primer and probe sequences as disclosed herein can be used as diagnostic assays for the detection of N7/N16 homoeologous chromosomal reciprocal translocation. As such, 1.5 ul of the ~6 ng/ul DNA is used in the assay mix, and 18 uM of each probe, and 4 uM of each primer is combined to make each assay. Next, 13.6 ul of the assay is combined with 1000 ul of ToughMix PCR Master Mix™ (Quanta Beverly, Mass, USA). A Meridian (LGC Genomics, Hoddesdon, Hertfordshire, UK) liquid handler dispenses 1.3 ul of the mix onto a 1536 plate containing ~6 ng of dried DNA. The plate is sealed with a Phusion™ laser sealer (LGC Genomics, Hoddesdon, Hertfordshire, UK) and thermocycled using a LGC Genomics™ Hoddesdon, Hertfordshire, UK hydrocycler with the following conditions: 94° C. for 15 min, 40 cycles of 94° C. for 30 secs, 60° C. for 1 min. PCR products are measured using at wavelengths 485 (FAM) and 520 (VIC) by a Pherastar plate reader (BMG Labtech, Offenburg, Germany). The values are normalized against ROX and plotted and scored on scatterplots utilizing the KRAKEN software (LGC Genomics, Hoddesdon, Hertfordshire, UK). Genotype is determined by the presence or absence of fluorescence specific to the translocation.

While the present disclosure may be susceptible to various modifications and alternative forms, specific embodiments have been described by way of example in detail herein. However, it should be understood that the present disclosure is not intended to be limited to the particular forms disclosed. Rather, the present disclosure is to cover all modifications, equivalents, and alternatives falling within the scope of the present disclosure as defined by the following appended claims and their legal equivalents.

What is claimed is:

1. A method of identifying the presence or absence of a N7/N16 homoeologous chromosomal reciprocal translocation in a canola plant, tissue, or cell, the method comprising:
(a) detecting in the canola plant, tissue, or cell at least one PCR amplicon associated with the N7/N16 homoeologous chromosomal reciprocal translocation, wherein the one or more PCR amplicons localize within either SEQ ID NO:2 or SEQ ID NO:4;
(b) screening the first canola plant, tissue, or cell for the N7/N16 homoeologous chromosomal reciprocal translocation; and
(c) selecting the first canola plant, tissue, or cell or selecting a progeny of the first canola plant, tissue, or cell whereby the resulting plant, tissue, or cell displays the N7/N16 homoeologous chromosomal reciprocal translocation.

2. The method of claim 1, wherein the amplicon of SEQ ID NO:2 comprises a primer associated with any one of SEQ ID NO:5493-8026, SEQ ID NO:9, SEQ ID NO:10, SEQ ID NO:14, SEQ ID NO:16 or a complement thereof.

3. The method of claim 2, wherein the amplicon of SEQ ID NO:2 comprises a forward or 5′ primer associated with any one of SEQ ID NO:5493-8026, SEQ ID NO:9, SEQ ID NO:10, SEQ ID NO:14, SEQ ID NO:16 or a complement thereof.

4. The method of claim 2, wherein the amplicon of SEQ ID NO:2 comprises a reverse or 3′ primer associated with any one of SEQ ID NO:5493-8026, SEQ ID NO:9, SEQ ID NO:10, SEQ ID NO:14, SEQ ID NO:16 or a complement thereof.

5. The method of claim 1, wherein the amplicon of SEQ ID NO:2 comprises a probe associated with any one of SEQ ID NO:372-488 or a complement thereof.

6. The method of claim 1, wherein the amplicon of SEQ ID NO:4 comprises a primer associated with any one of SEQ ID NO: 8027-10560, SEQ ID NO:11, SEQ ID NO:12, SEQ ID NO:18, SEQ ID NO:20 or a complement thereof.

7. The method of claim 6, wherein the amplicon of SEQ ID NO:4 comprises a forward or 5′ primer associated with any one of SEQ ID NO: 8027-10560, SEQ ID NO:11, SEQ ID NO:12, SEQ ID NO:18, SEQ ID NO:20 or a complement thereof.

8. The method of claim 6, wherein the amplicon of SEQ ID NO:4 comprises a reverse or 3′ primer associated with any one of SEQ ID NO: 8027-10560, SEQ ID NO:11, SEQ ID NO:12, SEQ ID NO:18, SEQ ID NO:20 or a complement thereof.

9. The method of claim 1, wherein the amplicon of SEQ ID NO:4 comprises a probe associated with any one of SEQ ID NO:255-371 or a complement thereof.

10. The method of claim 1, wherein the amplicon of SEQ ID NO:2 comprises a polynucleotide that shares at least 98% sequence identity with a primer associated with any one of SEQ ID NO:5493-8026, SEQ ID NO:9, SEQ ID NO:10, SEQ ID NO:14, SEQ ID NO:16 or a complement thereof.

11. The method of claim 1, wherein the amplicon of SEQ ID NO:2 comprises a polynucleotide that shares at least 95%, 96%, 97%, 98%, or 99% sequence identity with a primer associated with any one of SEQ ID NO:5493-8026, SEQ ID NO:9, SEQ ID NO:10, SEQ ID NO:14, SEQ ID NO:16 or a complement thereof.

12. The method of claim 1, wherein the amplicon of SEQ ID NO:2 comprises a probe that shares at least 95%, 96%, 97%, 98%, or 99% sequence identity with a probe associated with any one of SEQ ID NO:372-488 or a complement thereof.

13. The method of claim 1, wherein the amplicon of SEQ ID NO:4 comprises a polynucleotide that shares at least 95%, 96%, 97%, 98%, or 99% sequence identity with a primer associated with any one of SEQ ID NO: 8027-10560, SEQ ID NO:11, SEQ ID NO:12, SEQ ID NO:18, SEQ ID NO:20 or a complement thereof.

14. The method of claim 1, wherein the amplicon of SEQ ID NO:4 comprises a probe that shares at least 95%, 96%, 97%, 98%, or 99% sequence identity with a probe associated with any one of SEQ ID NO:255-371 or a complement thereof.

15. The method of claim 1, the method further comprising:
(a) quantitating the amplicon produced by an amplification reaction.

16. The method of claim 15, wherein quantitating the results of the amplification reaction comprises producing a signature profile.

17. The signature profile of claim 16, wherein the signature profile is selected from the group consisting of a melting temperature curve signature profile and a fluorescence signature profile.

18. The method of claim 17, wherein the signature profile is produced from an intercalating DNA dye, a cyanine dye, or a fluorescent dye.

19. The method of claim 18, wherein the fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

20. The method of claim 1, the method further comprising determining the presence or absence of the N7/N16 homoeologous chromosomal reciprocal translocation by determining the nucleotide sequence of said nucleic acid.

21. The method of claim 1, the method further comprising determining the presence or absence of the N7/N16 homoeologous chromosomal reciprocal translocation by determining the size of said nucleic acid.

22. The method of claim 21, wherein said determining the size comprises HPLC or electrophoresis.


SEQUENCE LISTING (1115 sequences)

SEQ ID NO: 1 - DNA, 5002 bp, Brassica napus
  source 1..5002: mol_type = genomic DNA
aattaagaaaaacatttgtataaaaatattttctaaaatatttttaatatgtgagcatatatattttaatgaattttttatatatatacaagtaatctgatatttgagttaaaatttttggaaacataaaatgctaatttcgaattatgtttgtgtgattatttgtaagaagattaagccaacatgtgcaggaaaaacacctggttaacaatgagaaaatgaaagatttggctctgttttacaggaataatgagctttctttaagcagcaattttaagcctccaacaagagaaaatcaaagttcatcggctaaactatatatctttacataacaagacggtttctgccgcatagtctttcagtttcgtgtcttgagaaacccaacctacaaaagttcctgctttattcttatttcttcacagcagaacactcgaccttttcacctgacagtagctgaagaccggaccagaaccctctaacgccatggacgacaacaacttatggtgggaagccagctaagtttggccccatgccctgttaagtagtcaataagaaccacataagaagactaatgtatcaacaacctcgttataattgtaattactccttaattaacaaaccaagtattcagcattacaaaagtatagtgacttggatgagaaaagcctcagttattatgtttgtgatggtttccacattttaacgttgtcacgtgaaacactagtacctaaggtacgcaaagtatcaaatattagaatccctatgtaccattttcttcaacatgatcttgttttagtgaaggctttcactgccgcacatgtgattacgttgaaaagtaaaggcctccacatgaaaaatctatcaatttacgtctatcaagccatgaaaaccagcctcaatacacattccctgcatagatgctactcttgaacatactaaactccacgaaactatggtattttctcatgtgacttgtcagctcgatacaagttttagatactaaaaactatcttaaccacaatccccttgacaattgcatctacagttaaagctaaataccttgtgaagcattgagattaactttaggaaacgataactagacgttagtactcgttttcagaaagtaccatctataaggcaaccactccagtgactgctacatctaccttaagggcaaacttcaagcagggacaagtattgcacagaaactacgttagatacagtagctgactatacccgatactgaacacactaagagggtccagaagctgtgactgttgttgctgttgcatgtcttccaactgcggaaacgccaaatcagctcctgaactcacagccccagcactagacggcgtccctttcgcttcaatgagccgccacatgtaccaagacccaacagacctatacggacgccacttctcgcaaatcccctccatctgcgaaggacgaggcagctccggcaagtcataaagcatctgcacacctttcctcacaccaagatcgttaacaggcaacacatcaggtctatgaagcgagtttatcataaacatatgaaccgaccacgacccaatcccgttaaccatcgtcagcatcgtgaacagagacttatcatccatgctcacaatcccagaatccgacaagataccgttttgatactttctcgcgagatcgtggaggtaactcgctttcctcccggagacaccaatctgacgaagctcctgaggcgtcaaaggcaagacattctccggaacgacgccgttttcgcctccgcagagggaaacgaagcgcgtgtagattgagttccccgctttggcagcaagctgctggtagaggatgcttcggatgagagccaagaagggagtgttgaaagactcgtactttggcggtgggtgtgcgtcgatcaacgaggctaggagaggatcgacgctgcggaggtgggtaatcgccgcttcgagctctccctcgcaagttagagactttgcgtggatcctcggtggctgcgatggtttgcccttcggtgaaggtctcgcgtcgttgttgggagaaagctttctgatttttcgaggacggagagggattttggaaggaggggaggatacattgcttagctcggtgattctcggggcttcgatggtggttgatgagacgattgagccgggaacggaggcggaggaggagtcgtcggaatcgttgctttgcggtggttgagaaggttgtggttcacccatgtggaaaatcgaattgggttgaattagggttttgtttgatcgcttgaagaagtaatcggagaatggacacggcggagtgaagttgcgtcttttggcttgactttgattcttttctgacgcgcttttaactatagtggccttcacgctccgatgtcttctgtgtgggctttatgataaaaaagtagagtccgtttgctttaataagcccaaactggattacttgggtctttcaactgagctcaatatacacaagaaacaccttactttgttaaatttaaaatagttacaaagtaaataaaagttgattaaaactccctctgaaaactggggggaaaagtcgttgatctattgttttcatataatttgagaattaactgagtgatgtttttgtggtcaacaaaagagttaattaatgggaaattgcatccaatatacacacaaaaaaacaaaattcactaactaactaaaaacaccccctctctcctactttctcttcctatctctctctactctctctctctctaaaaatctaatttcactttttttttggctattcagcaaataagcccttaattaattgggtgatgaataagccgaaactggaatgcttgagttagacccttctttgtgaaataaataacaattaaatacttttccaagagtcttgatctattgttttcatataatttgagactttactgagtaatgttatgtttaattttattataaggattaaagaataaaatatacaatggcgatgggaaggactattcactacaaaaaaaacattgaattgtatcacttaaattgtatcacaaaaataagtgataccattttaaatcacttattcagaactgaaacaaatatttatacttttaaatcatttatagtaattgatgttattattaattaatttaacatcagttcgatataagtgatgtcttttcttttaatttgtatcattttaacaaaaatgatacaattatataaaattatatcaattacacaattgatgcatatattattttgtttagtatcacttatttaaatgatctataatttacatcagtaaaataattgatgctaataaaaattatatatatatatatatatatattagcatcggtctaataaaaatgatattgtttttaatcttattttgcatctgttgaaatatattatctagtacaaaattagtttggatctataaaataattaataaatataatataattgtataattatatactaaacaggatctattatagttccccaaaaaaaggaaccacaatcccacataaaacgtgaactaatcagttaaaaagtagggaataccagaaatagtataatattcttgtgattttaattttttgttagctttaattttactggatcataattttctataactgtttcacttcaatattttcatcttcgtctctttttcattttttctctgtttcagtttcttaaacttttgttcagagcttaaaatttgttgttttggttatttgatcagaaacagatttcaacaatatcaagcattatatgagtcacgatgacaaatgcgtttcttttcatgaaagggtttaaagattgctgaagtttgctaatcaaacgactctgattaaacttttgagaggtattatttcttttttctttctctattctcaaactttttgaaaactacaagttcttatattttttgtttatgtcttaataatcattctctattaggatcagaataattcagagatatatgaagacacgacaagagtgatttgaaatgtggaggaaaatcgatagagattgaagatgaaaaaagaagatgcaaagaatgagaagaaagaagaagaatctggatataattgctagtattttttttgtatctccgcattcttcatgtttttttctttctcataactagtaatttgtttaactttctatagtctttttttcttctctgtaccttttttatttttcttgttttcttggctacgttatatttcaatggaaaattaataaattgttacaaaataatattttattttatatgtattttctactctacaaatgattctaatatttaaaacaataactataaatagtaaaactattaaaatattttttaataaatcgatattaatttattaaattattaattaatattttattaactaaatcagttattttaattgatgtcaatactaatatcatttggaaaaattgatgtaattgttaatatcgtttttacaattgatatatatattgacatcacataaagtaactgatgaaaatataataatttcttacatcatttgttaataaaatgatgtaaattatttgtatcaccactttcagagtcatttatttaagtgatgtaaaattattttacatcatttataactaatgcaaaaatataaattaaatgatgctaaatgacttttttttttgtagtgattgtacatgtgaggaacgaggaggaaacaaaaagacttgatcagtgttatgtacaatgcagatatataaatatttgtggtcaacaaaaaaaattagatcgtggtgatgtggagacagtaatatcagatcacaaagacgtgtttggtgtgatatgatactctgtctttctgaccatctcttacctcacatttcgctcgttttgttttttgttcttttatttctttctaccaaacagcaaactaaaaaacaatgaaaataaatattttttttatttaatataaagtctaccatctttaaaaataatattctggctt

SEQ ID NO: 2 - DNA, 5002 bp, Brassica napus
  source 1..5002: mol_type = genomic DNA
aattaagaaaaacatttgtataaaaatattttctaaaatatttttaatatgtgagcatatatattttaatgaattttttatatatatacaagtaatctgatatttgagttaaaatttttggaaacataaaatgctaatttcgaattatgtttgtgtgattatttgtaagaagattaagccaacatgtgcaggaaaaacacctggttaacaatgagaaaatgaaagatttggctctgttttacaggaataatgagctttctttaagcagcaattttaagcctccaacaagagaaaatcaaagttcatcggctaaactatatatctttacataacaagacggtttctgccgcatagtctttcagtttcgtgtcttgagaaacccaacctacaaaagttcctgctttattcttatttcttcacagcagaacactcgaccttttcacctgacagtagctgaagaccggaccagaaccctctaacgccatggacgacaacaacttatggtgggaagccagctaagtttggccccatgccctgttaagtagtcaataagaaccacataagaagactaatgtatcaacaacctcgttataattgtaattactccttaattaacaaaccaagtattcagcattacaaaagtatagtgacttggatgagaaaagcctcagttattatgtttgtgatggtttccacattttaacgttgtcacgtgaaacactagtacctaaggtacgcaaagtatcaaatattagaatccctatgtaccattttcttcaacatgatcttgttttagtgaaggctttcactgccgcacatgtgattacgttgaaaagtaaaggcctccacatgaaaaatctatcaatttacgtctatcaagccatgaaaaccagcctcaatacacattccctgcatagatgctactcttgaacatactaaactccacgaaactatggtattttctcatgtgacttgtcagctcgatacaagttttagatactaaaaactatcttaaccacaatccccttgacaattgcatctacagttaaagctaaataccttgtgaagcattgagattaactttaggaaacgataactagacgttagtactcgttttcagaaagtaccatctataaggcaaccactccagtgactgctacatctaccttaagggcaaacttcaagcagggacaagtattgcacagaaactacgttagatacagtagctgactatacccgatactgaacacactaagagggtccagaagctgtgactgttgttgctgttgcatgtcttccaactgcggaaacgccaaatcagctcctgaactcacagccccagcactagacggcgtccctttcgcttcaatgagccgccacatgtaccaagacccaacagacctatacggacgccacttctcgcaaatcccctccatctgcgaaggacgaggcagctccggcaagtcataaagcatctgcacacctttcctcacaccaagatcgttaacaggcaacacatcaggtctatgaagcgagtttatcataaacatatgaaccgaccacgacccaatcccgttaaccatcgtcagcatcgtgaacagagacttatcatccatgctcacaatcccagaatccgacaagataccgttttgatactttctcgcgagatcgtggaggtaactcgctttcctcccggagacaccaatctgacgaagctcctgaggcgtcaaaggcaagacattctccggaacgacgccgttttcgcctccgcagagggaaacgaagcgcgtgtagattgagttccccgctttggcagcaagctgctggtagaggatgcttcggatgagagccaagaagggagtgttgaaagactcgtactttggcggtgggtgtgcgtcgatcaacgaggctaggagaggatcgacgctgcggaggtgggtaatcgccgcttcgagctctccctcgcaagttagagactttgcgtggatcctcggtggctgcgatggtttgcccttcggtgaaggtctcgcgtcgttgttgggagaaagctttctgatttttcgaggacggagagggattttggaaggaggggaggatacattgcttagctcggtgattctcggggcttcgatggtggttgatgagacgattgagccgggaacggaggcggaggaggagtcgtcggaatcgttgctttgcggtggttgagaaggttgtggttcacccatgtggaaaatcgaattgggttgaattagggttttgtttgatcgcttgaagaagtaatcggagaatggacacggcggagtgaagttgcgtcttttggcttgactttgattcttttctgacgcgcttttaactatagtggccttcacgctccgatgtcttctgtgtgggctttatgataaaaaagtagagtccgtttgctttaataagcccaaactggattacttgggtctttcaactgagctcaatatacacaagcaacaccctactttgtgaaaagtcgctgatcttattgttttcaaataatttgagaattaactgagtgatgtttttgtggtcaacaaaaaagttaattaattgggagatgaataaaactggaatgcttaattaatttaaaagaaattttaatgaaacaaaccctcaactataactataggcccatttgttttttaacccttaagcatttttataattgttagagataatatcaaaagatacgattaaccatttgctatttaggataaatattatcttttttttattttgtcatttcttatgtcagttaatttgaccgacttactaccactataaatagtggtttcatctcatttataacgatatgagaacaacaataataatatgatcatctcacatattcatctctcttacgttacttagcaaacaagaaacaaatatatatatatatatatatatatatgtaataatacatatataaatattcacctattatttcacaacacgttatcaacacgagctctcatataccggtgaagtctatcaatccggaagctctaaccaaccgaagcttatcaatccgaaagttcttaaaccagccgagaaaatgccaaactttgagactatggatggagatttgtgtttggcagatagtgcgtcaacgcacacgataattaaagataagaaatatttctccagtctcaaaataaaagattacgctggaagcgtaagtacaatatctggtaatgcaaagattattatgagctctggaagagcgaaattttcaatgccagggggaacaatatttgaaataagtgatgcattgtatccccccgagtctcatagaaacttattaagttttaaggatatccgaagaaatggatatcatattgagactatgagtaaagatgacactaaatttctttgcaataagtccgagaggaaacatatattggaagagttaagaatgttatcctctggactatattgtacgaaaattaccatgactgagtcctatgccgtggtaaacatgaagtttactgatacatttaaaatttggcatgagaggctaggacatcctggttcagtcatgatgagaaagatagtgtaaaattcgaatggccatccgttgaaaaacggaaaatttttgcaaacgggcgagtttacatgtaatgcatgttctcaaggaaaacttataactcggccatcaccagcaaaagtaggcaatgaatcaccaatgtttttggaaagaatacatggtgatatatgtgggccgatccacccaccgtgcgggccgtttaagtattttatggtattgattgacgcatatagtagatggtcaagtgtatctcttttgacgacacaaaatacggctttcgcaaagttcattgcccagataataaagctgagaacgcagttctcagagtatgccattaagaaagtaaggcttgataatgctggtgaatttacatcgcaagcctttgatgattattgtatgtcaatggggattgatgtggagcatccagtttcccatgtgcatacacaaaatggcatggccgagtcattgataaaacagttgcagttgattgcaagaccgttagtcttgagaacgaagctcacaatttatgtatggggtcatgctatattgcatgcagctgcactggttcggttaagaccgagtgcatatcataaatactccccattacgattggcatctgggcaagagctagatgtatcccatctccgtgtctttgggtgtgcggtctatgttccgatagcaccgccacaaagaacaaaaatggacccacaaaggagattgagaatatatgtgggttatacgtcaccaagtataataagatatctggaaccagtaactggtgatatgtttatgacacgatttaccaattgccattttaatgaggatgaatttccagcgttaggggaaggaattaatcgaattcctaaagagattacttggtgtacaccgtcgttgttacactttgatccccttacgaatcaaagagagctggaagttcagaaaattgtgcatttgcataatttggcaaacaagttaccagatgcgttcacagataagagaagggtgacgaagtcatgtatacccgctgaaaatgttccatcaagagttgatgttcctagagaacaaactgatggaaacaaaatgagtgaacctcgagttcagttaaagaggggaaggtcagcgggttctaaaaataaaaatccccaaaagaaaaagaaattggatgaacaaagtaaagttccagaagagcctgatatcgaaaaacaaaagataatggtttaaggagatgttttttaaatgagaatatttagttttaaaactcgttttcagcttattcaagttctaaagttttattgatcttataaaaaattgaaagtttgttcgtagaaactaaggaatgacaatgataatgcttgttcacgttcaagtcttaaattttagaatgaaagatttgctttcttaggttagttatgaagatgattgtcg

SEQ ID NO: 3 - DNA, 5002 bp, Brassica napus
  source 1..5002: mol_type = genomic DNA
agtttgttctctaggaacatcaactcttgatggaacattttcagcgggtatacatgacttcgtcacccttctcttatctgtgaacgcatctggtaacttgtttgccaaattatgcaaatgcacaattttctgaacttccagctctctttgattcgtaaggggatcaaagtgtaataacgacggtgtacaccaagtaatctctttaggaattcgattaattccttcccctaacgctggaaattcatcctcattaaaatggcaattggcaaatcgtgtcgtaaacatatcaccagttactggttccagatatcttattatacttggtgacgtataacccacatatattctcaatctcctttgtgggtccatttttgttctttgtggcggtgctatcggaacatagaccgcacacccaaagacacggagatgggatacatctagctcttgcccagatgccaattgtaatggggagtatttatgatatgcactcggtcttaaccgaaccagtgcagctgcatgcaatatagcatgaccccatacagaaattgtgagcttcgttctcaagactaacggtcttgcaatcaactgcaaccgttttatcaatgactcggccatgccattttgtgtatgcacatgggaaactggatgctccacatcaatccccattgacatacaataatcatcaaaggcttgcgatgtaaattcaccagcattatcaagccttactttcttaatggcatactctgagaactgcgttctcagctttattatctgggcaatgaactttgcgaaagccgtatttcgtgtcgtcaaaagagacacacttgaccatctactagatgcgtcaatcaataccatgaaatacttaaacggcccgcacggtgggtggatcggcccacatatatcaccatgtattctttccaaaaacattggtgattcattgcctacttttgctggtgatggccgagttataagttttccttgagaacatgcattacatgtaaactcgcccgtttgcaaaatttttccgtttttcaacggatggccattcgaattttgcactatctttctcatcatgactgaaccaggatgtcctagcctctcatgccaaattttaaatgtatcagtaaacttcatgtttaccacggcataggactcagtcatggttattttcgtacaatatagtccagaggataacattcttaattcttccaatatatgtttcctctcggacttaatgcaaagaaattcaatgccatctttactcatagtctcaatatgatatccatttcttcggatatccttaaaacttaataagtttctatgagactcggggggatacaatgcatcacttatttcaaatattgttccccctggcattgaaaatttcgctcttccagagcccataataatctttgcattactagatattgtacttacgcttccagcgtaatcttttattttgagactggaaaaatatttcttatctttaattatcgtgtgcgttgacgcactatctgccaaacacaaatctccatccatagtctcaaagtttggcattttctcggctggtttaagaactttcggattgataagcttcggttggttagagcttccggattgatagacttcaccggtatatgagagctcgtgttgataacgtgttgtgaaataataggtgaatatttatatatgtattattacatatatatatatatatatatatatatatttgtttcttgtttgcaaagtaacgtaagagagaggaatatgtgagatgatcatattattattgttgttctcatatcgttataaatgagatgaaaccactatttatagtggtagtaagtcggtcaaattaactgacataagaaaagacaaaataagaaaagataatatctatagcaaatggttaatcgtatcttttgatattatctctaacaattataaaaatgcttaagggttaaaaaacaaatgggcctatagttatagttgagggtttgtttcattaaaatctcttttaaattaattaagcattccagttttattcatctcccaattaattaacttttttgttgaccacaaaaacatcactcggttaattctcaaattatttgaaaacaataagatcaacgacttttcacaaagtagggtgtttcttgtgtatattgagttcagttgaaagacccaagtaatcaagtctgggcttattaaagcacacgggctctactttttattataaagcccacacagaaaatgtcggagcgtgagctccactttagttaaaagcgcgtcagaaaataatcaaagtcaagcccaaagacgcaacttgactccgccgtgtccattctccgataaggtgttttcgattcttcttcgtctctcttcaagcgatcaaaaccctaattcaacccaattcgatttccacatgggtgaacctcaacctcaaccccaatcacccaaacccgaaaaccgtaatccaatcccaccgcaaaacaacaacgactcctcctcctcctccgcctccgttcccggctcaatcgtctcatcaaccaccatcgaagccccgagaatcaccgagctaagcaatgtatcctcctcccctccttccaaaatcccactccgtcctcgaaaaatcagaaagctttctcccaacaacgacgcggtgacgtcatcgagaccttcagcgaagggcaaaccatcgcagccaccgaggatccacgcaaagtctctaacttgcgagggagagctcgaagcggcgataacccacctccgcagcgtcgatcctctgctagcgtcgttgatcgacgcacacccaccgccaaagtacgagtctttcaacactcccttcttggctctgatacgaagcattctctaccagcagctagctgctaaagcggggaactcaatctacacacgcttcgtttccctctgcggcggcgaaaacggcgtcgttccggagaatgtcttgcctttgacgcctcaggagcttcgtcagattggtgtctctgggaggaaggcgagttacctccacgatcttgcgaggaagtatcaaaacgggatcttgtcggattctgggatattgaacatggatgataagtctctgttcacgatgctgacgatggttaacgggatcggttcgtggtcggttcatatgtttatgatgaactcgcttcatagacctgatgtgttgcctgttaacgatcttggtgtgaggaaaggtgtgcagatgctttatgacttgccagagttgcctcgtccttcgcagatggagggtctttgcgagaagtggcgtccgtataggtctgttgggtcgtggtacatgtggcggctcattgaagctaaagggactccgtcgaatgctggagcggtgagttcaggagctgatttggcgtttccgcagttggaagacttgcaacagcaacatcagcaacaacagtcacagcttctggaccctcttagtgtgttcagtatcgggtatagtcagctactgtatctaacgtagtttctctgcaatacttgtccctgcttttaccttgcttgaagtttgcccttaaggtagatatagcagtcactggagtggttgccttatagatggtactttctgaaaacgagtactaacgtctagttatcgtttccttaagttaatctcaatgcttcaaggtatttagctttaactgtagatgcaattgtcaaggggattgtggttaagatagtttttagtttattttgtcatctaaaacttgtatcgagctgacaagtcgcatgaaaaaataccatagtttcgtggagtttagtatgttcaagagtagcatctatgcagggaatgtgtattgaggctggttttcatggcttgatagacgtatggatattaattgatagacttttcatgtggagtccttcgcttttcaacgtaatcacatgtgcggcagtgaaagccttcactaaaacaagatcatgttgaagaaaatggtgcataggggttctaatatttgatactttgcgtaccttaggtactaatgtttcacgtgacaacgttaaaatgtggaaaactcaagtgaaagtgaatagtttgcagtgggttttaaccatcacaacataataactgaagcttttctcatccaagtcactatacttttgtaatgctaaatacttggtttgttaattaaggagtaattacaattataacgaggttgttgatacattagtcttcttatgcggttcttattgactacttaacagggcatggggccaaacttagctggcttcccaccataagttgttgtcgtccatggcgttagagggattctggtccagtcttcagatactgtcagatgaaaaggtcgagtctgctgtgaagaaatacgaataaagcaggaactttgtaggttgggtttcttaagacacaaaactgaaagactatgcagcagaaaccctcttagtatgtaagatatagtttagctgatgaactttgattttctcttgttggagacttaaaattgctgtttaaaaaaactcataattcatgtaaaacagagtcaaatctttcattttctcattgcttaaccagttgttttgcgggcataatcttctttgcataatcttcttacaatttttttttactaatgaattttcgcacgcgtaaatatatttttttccaaatatttattagtttatgttttattaatttgaaattagcattttatgtttccaaaatttttaaatcaaacatcaaattacttatatataaaaaagaattcactaaaatatatatgttcacatattaaaaatattttagaaaatgtttttatacaaatgatttttttaattaataattaattataatttgttttatatattaattttaaaaaatttcataatagaaaatattatttcaagataacaacacaattaagatttaggaaaaattggcaaattgggcaaattgcaagtttgatattagggagttggacgacctcgagtttaaattagaagattggac

SEQ ID NO: 4 - DNA, 5002 bp, Brassica napus
  source 1..5002: mol_type = genomic DNA
acaaaaaaaaaagtcatttagcatcatttaatttatatttttgcattagttataaatgatgtaaaataattttacatcacttaaataaatgactctgaaagtggtgatacaaataatttacatcattttattaacaaatgatgtaagaaattattatattttcatcagttactttatgtgatgtcaatatatatatcaattgtaaaaacgatattaacaattacatcaatttttccaaatgatattagtattgacatcaattaaaataactgatttagttaataaaatattaattaataatttaataaattaatatcgatttattaaaaaatattttaatagttttactatttatagttattgttttaaatattagaatcatttgtagagtagaaaatacatataaaataaaatattattttgtaacaatttattaattttccattgaaatataacatagccaagaaaacaagaaaaataaaaaaggtacagagaagaaaaaaagaatatagaaagttaaacaaattactagttatgagaaagaaaaaaacatgaagaatgcggagatacaaaaaaaatactagcaattatatccagattcttcttttttcttctcattctttgcatcttcttttttcatcttcaatctctatcgattttcctccacatttcaaatcactcttgtcgtgtcttcatatatctctgaattattctgatcctaatagagaatgattattaagacataaacaaaaaatataagaacttgtagttttcaaaaaatttgagaatagagaaagaaaaaagaaataatacctctcaaaagtttaatcagagtcgtttgattagcaaacttcagcaatctttaaaccctttcatgaaaagaaacgcatttgtcatcgtgactcatataatgcttgatattgttgaaatctgtttctgatcaaataaccaaaacaacaaattttaagctctgaacaaaagtttaagaaactgaaacagagaaaaaatgaaaaagagacgaagatgaaaatattgaagtgaaacagttatagaaaattatgatccagtaaaattaaagctaacaaaaaattaaaatcacaagaatattatactatttctggtattccctactttttaactgattagttcacgttttatgtgggattgtggttcctttttttggggaactataatagatcctgtttagtatataattatacaattatattatatttattaattattttatagatccaaactaattttgtactagataatatatttcaacagatgcaaaataagattaaaaacaatatcatttttattagaccgatgctaatatatatatatatatatatataatttttattagcatcaattattttactgatgtaaattatagatcatttaaataagtgatactaaacaaaataatatatgcatcaattgtgtaattgatataattttatataattgtatcatttttgttaaaatgatacaaattaaaagaaaagacatcacttatatcgaactgatgttaaattaattaataataacatcaattactataaatgatttaaaagtataaatatttgtttcagttctgaataagtgatttaaaatggtatcacttatttttgtgatacaatttaagtgatacaattcaatgttttttttgtagtgaatagtccttcccatcgccattgtatattttattctttaatccttataataaaattaaacataacattactcagtaaagtctcaaattatatgaaaacaatagatcaagactcttggaaaagtatttaattgttatttatttcacaaagaagggtctaactcaagcattccagtttcggcttattcatcacccaattaattaagggcttatttgctgaatagccaaaaaaaaagtgaaattagatttttagagagagagagagtagagagagataggaagagaaagtaggagagagggggtgtttttagttagttagtgaattttgtttttttgtgtgtatattggatgcaatttcccattaattaactcttttgttgaccacaaaaacatcactcagttaattctcaaattatatgaaaacaatagatcaacgacttttccccccagttttcagagggagttttaatcaacttttatttactttgtaactattttaaatttaacaaagtaaggtgtttcttgtgtatattgagctcagttgaaagacccaagtaatccagtttgggcttattaaagcaaacggactctacttttttatcataaagcccacacagaagacatcggagcgtgaaggccactatagttaaaagcgcgtcagaaaagaatcaaagtcaagccaaaagacgcaacttcactccgccgtgtccattctccgattacttcttcaagcgatcaaacaaaaccctaattcaacccaattcgattttccacatgggtgaaccacaaccttctcaaccaccgcaaagcaacgattccgacgactcctcctccgcctccgttcccggctcaatcgtctcatcaaccaccatcgaagccccgagaatcaccgagctaagcaatgtatcctcccctccttccaaaatccctctccgtcctcgaaaaatcagaaagctttctcccaacaacgacgcgagaccttcaccgaagggcaaaccatcgcagccaccgaggatccacgcaaagtctctaacttgcgagggagagctcgaagcggcgattacccacctccgcagcgtcgatcctctcctagcctcgttgatcgacgcacacccaccgccaaagtacgagtctttcaacactcccttcttggctctcatccgaagcatcctctaccagcagcttgctgccaaagcggggaactcaatctacacacgcttcgtttccctctgcggcggcgaaaacggcgtcgttccggagaatgtcttgcctttgacgcctcaggagcttcgtcagattggtgtctctgggaggaaggcgagttacctccacgatcttgcgaggaagtatcaaaacgggatcttgtcggattctgggatattgaacatggatgataagtctctgttcacgatgctgacgatggttaacgggatcggttcgtggtcggttcatatgtttatgatgaactcgcttcatagacctgatgtgttgcctgttaacgatcttggtgtgaggaaaggtgtgcagatgctttatgacttgccagagttgcctcgtccttcgcagatggagggtctttgcgagaagtggcgtccgtataggtctgttgggtcgtggtacatgtggcggctcattgaagctaaagggactccgtcgaatgctggagcggtgagttcaggagctgatttggcgtttccgcagttggaagacttgcaacagcaacatcagcaacaacagtcacagcttctggaccctcttagtgtgttcagtatcgggtatagtcagctactgtatctaacgtagtttctctgcaatacttgtccctgcttttaccttgcttgaagtttgcccttaaggtagatatagcagtcactggagtggttgccttatagatggtactttctgaaaacgagtactaacgtctagttatcgtttccttaagttaatctcaatgcttcaaggtatttagctttaactgtagatgcaattgtcaaggggattgtggttaagatagtttttagtttattttgtcatctaaaacttgtatcgagctgacaagtcgcatgaaaaaataccatagtttcgtggagtttagtatgttcaagagtagcatctatgcagggaatgtgtattgaggctggttttcatggcttgatagacgtatggatattaattgatagacttttcatgtggagtccttcgcttttcaacgtaatcacatgtgcggcagtgaaagccttcactaaaacaagatcatgttgaagaaaatggtgcataggggttctaatatttgatactttgcgtaccttaggtactaatgtttcacgtgacaacgttaaaatgtggaaaactcaagtgaaagtgaatagtttgcagtgggttttaaccatcacaacataataactgaagcttttctcatccaagtcactatacttttgtaatgctaaatacttggtttgttaattaaggagtaattacaattataacgaggttgttgatacattagtcttcttatgcggttcttattgactacttaacagggcatggggccaaacttagctggcttcccaccataagttgttgtcgtccatggcgttagagggattctggtccagtcttcagatactgtcagatgaaaaggtcgagtctgctgtgaagaaatacgaataaagcaggaactttgtaggttgggtttcttaagacacaaaactgaaagactatgcagcagaaaccctcttagtatgtaagatatagtttagctgatgaactttgattttctcttgttggagacttaaaattgctgtttaaaaaaactcataattcatgtaaaacagagtcaaatctttcattttctcattgcttaaccagttgttttgcgggcataatcttctttgcataatcttcttacaatttttttttactaatgaattttcgcacgcgtaaatatatttttttccaaatatttattagtttatgttttattaatttgaaattagcattttatgtttccaaaatttttaaatcaaacatcaaattacttatatataaaaaagaattcactaaaatatatatgttcacatattaaaaatattttagaaaatgtttttatacaaatgatttttttaattaataattaattataatttgttttatatattaattttaaaaaatttcataatagaaaatattatttcaagataacaacacaattaagatttaggaaaaattggcaaattgggcaaattgcaagtttgatattagggagttggacgacctcgagtttaaattagaagattggacgaatcgccttttccagacttg

SEQ ID NO: 5 - DNA, 26 bp, Brassica napus
  source 1..26: mol_type = genomic DNA
tttaacaaagtaaggtgtttcttgtg

SEQ ID NO: 6 - DNA, 20 bp, Brassica napus
  source 1..20: mol_type = genomic DNA
ttaagccaacatgtgcagga

SEQ ID NO: 7 - DNA, 22 bp, Brassica napus
  source 1..22: mol_type = genomic DNA
agcgacttttcacaaagtaggg

SEQ ID NO: 8 - DNA, 20 bp, Brassica napus
  source 1..20: mol_type = genomic DNA
tgatggttaaaacccactgc

SEQ ID NO: 9 - DNA, 23 bp, Brassica napus
  source 1..23: mol_type = genomic DNA
caacgacttttcacaaagtaggg

SEQ ID NO: 10 - DNA, 25 bp, Brassica napus
  source 1..25: mol_type = genomic DNA
gcctcagttattatgtttgtgatgg

SEQ ID NO: 11 - DNA, 21 bp, Brassica napus
  source 1..21: mol_type = genomic DNA
gtgatggttaaaacccactgc

SEQ ID NO: 12 - DNA, 26 bp, Brassica napus
  source 1..26: mol_type = genomic DNA
tttaacaaagtaaggtgtttcttgtg

SEQ ID NO: 13 - DNA, 24 bp, Brassica napus
  source 1..24: mol_type = genomic DNA
gtccgtttgctttaataagcccaa

SEQ ID NO: 14 - DNA, 24 bp, Brassica napus
  source 1..24: mol_type = genomic DNA
tcccgtgtgctttaataagcccag

SEQ ID NO: 15 - DNA, 21 bp, Brassica napus
  source 1..21: mol_type = genomic DNA
agatcagcgacttttcacaaa

SEQ ID NO: 16 - DNA, 21 bp, Brassica napus
  source 1..21: mol_type = genomic DNA
ccagttttcagagggagtttt

SEQ ID NO: 17 - DNA, 21 bp, Brassica napus
  source 1..21: mol_type = genomic DNA
ccagttttcagagggagtttt

SEQ ID NO: 18 - DNA, 21 bp, Brassica napus
  source 1..21: mol_type = genomic DNA
ggaggaggaggaggagtcgtt

SEQ ID NO: 19 - DNA, 25 bp, Brassica napus
  source 1..25: mol_type = genomic DNA
gggattggattacggttttcgggtt

SEQ ID NO: 20 - DNA, 24 bp, Brassica napus
  source 1..24: mol_type = genomic DNA
attttccacatgggtgaaccacaa

SEQ ID NO: 21 - DNA, 13 bp, Brassica napus
  source 1..13: mol_type = genomic DNA
ggaggaggaggag

SEQ ID NO: 22 - DNA, 13 bp, Brassica napus
  source 1..13: mol_type = genomic DNA
ggagtcgttgttg

SEQ ID NO: 23 - DNA, 13 bp, Brassica napus
  source 1..13: mol_type = genomic DNA
gagtcgttgttgt

SEQ ID NO: 24 - DNA, 13 bp, Brassica napus
  source 1..13: mol_type = genomic DNA
agtcgttgttgtt

SEQ ID NO: 25 - DNA, 13 bp, Brassica napus
  source 1..13: mol_type = genomic DNA
gtcgttgttgttt

SEQ ID NO: 26 - DNA, 13 bp, Brassica napus
  source 1..13: mol_type = genomic DNA
tcgttgttgtttt

SEQ ID NO: 27 - DNA, 13 bp, Brassica napus
  source 1..13: mol_type = genomic DNA
cgttgttgttttg

SEQ ID NO: 28 - DNA, 13 bp, Brassica napus
  source 1..13: mol_type = genomic DNA
gttgttgttttgc

SEQ ID NO: 29 - DNA, 13 bp, Brassica napus
  source 1..13: mol_type = genomic DNA
ttgttgttttgcg

SEQ ID NO: 30 - DNA, 13 bp, Brassica napus
  source 1..13: mol_type = genomic DNA
tgttgttttgcgg

SEQ ID NO: 31 - DNA, 13 bp, Brassica napus
  source 1..13: mol_type = genomic DNA
gttgttttgcggt

SEQ ID NO: 32 - DNA, 13 bp, Brassica napus
  source 1..13: mol_type = genomic DNA
gaggaggaggagt

SEQ ID NO: 33 - DNA, 13 bp, Brassica napus
  source 1..13: mol_type = genomic DNA
ttgttttgcggtg

SEQ ID NO: 34 - DNA, 13 bp, Brassica napus
  source 1..13: mol_type = genomic DNA
tgttttgcggtgg

SEQ ID NO: 35 - DNA, 13 bp, Brassica napus
  source 1..13: mol_type = genomic DNA
gttttgcggtggg

SEQ ID NO: 36 - DNA, 13 bp, Brassica napus
  source 1..13: mol_type = genomic DNA
aggaggaggagtc

SEQ ID NO: 37 - DNA, 13 bp, Brassica napus
  source 1..13: mol_type = genomic DNA
ggaggaggagtcg

SEQ ID NO: 38 - DNA, 13 bp, Brassica napus
  source 1..13: mol_type = genomic DNA
gaggaggagtcgt

SEQ ID NO: 39 - DNA, 13 bp, Brassica napus
  source 1..13: mol_type = genomic DNA
aggaggagtcgtt

SEQ ID NO: 40 - DNA, 13 bp, Brassica napus
  source 1..13: mol_type = genomic DNA
ggaggagtcgttg

SEQ ID NO: 41 - DNA, 13 bp, Brassica napus
  source 1..13: mol_type = genomic DNA
gaggagtcgttgt

SEQ ID NO: 42 - DNA, 13 bp, Brassica napus
  source 1..13: mol_type = genomic DNA
aggagtcgttgtt

SEQ ID NO: 43 - DNA, 14 bp, Brassica napus
  source 1..14: mol_type = genomic DNA
ggaggaggaggagt

SEQ ID NO: 44 - DNA, 14 bp, Brassica napus
  source 1..14: mol_type = genomic DNA
ggagtcgttgttgt

SEQ ID NO: 45 - DNA, 14 bp, Brassica napus
  source 1..14: mol_type = genomic DNA
gagtcgttgttgtt

SEQ ID NO: 46 - DNA, 14 bp, Brassica napus
  source 1..14: mol_type = genomic DNA
agtcgttgttgttt

SEQ ID NO: 47 - DNA, 14 bp, Brassica napus
  source 1..14: mol_type = genomic DNA
gtcgttgttgtttt

SEQ ID NO: 48 - DNA, 14 bp, Brassica napus
  source 1..14: mol_type = genomic DNA
tcgttgttgttttg

SEQ ID NO: 49 - DNA, 14 bp, Brassica napus
  source 1..14: mol_type = genomic DNA
cgttgttgttttgc

SEQ ID NO: 50 - DNA, 14 bp, Brassica napus
  source 1..14: mol_type = genomic DNA
gttgttgttttgcg

SEQ ID NO: 51 - DNA, 14 bp, Brassica napus
  source 1..14: mol_type = genomic DNA
ttgttgttttgcgg

SEQ ID NO: 52 - DNA, 14 bp, Brassica napus
  source 1..14: mol_type = genomic DNA
tgttgttttgcggt

SEQ ID NO: 53 - DNA, 14 bp, Brassica napus
  source 1..14: mol_type = genomic DNA
gttgttttgcggtg

SEQ ID NO: 54 - DNA, 14 bp, Brassica napus
  source 1..14: mol_type = genomic DNA
gaggaggaggagtc

SEQ ID NO: 55 - DNA, 14 bp, Brassica napus
  source 1..14: mol_type = genomic DNA
ttgttttgcggtgg

SEQ ID NO: 56 - DNA, 14 bp, Brassica napus
  source 1..14: mol_type = genomic DNA
tgttttgcggtggg

SEQ ID NO: 57 - DNA, 14 bp, Brassica napus
  source 1..14: mol_type = genomic DNA
aggaggaggagtcg

SEQ ID NO: 58 - DNA, 14 bp, Brassica napus
  source 1..14: mol_type = genomic DNA
ggaggaggagtcgt

SEQ ID NO: 59 - DNA, 14 bp, Brassica napus
  source 1..14: mol_type = genomic DNA
gaggaggagtcgtt

SEQ ID NO: 60 - DNA, 14 bp, Brassica napus
  source 1..14: mol_type = genomic DNA
aggaggagtcgttg

SEQ ID NO: 61 - DNA, 14 bp, Brassica napus
  source 1..14: mol_type = genomic DNA
ggaggagtcgttgt

SEQ ID NO: 62 - DNA, 14 bp, Brassica napus
  source 1..14: mol_type = genomic DNA
gaggagtcgttgtt

SEQ ID NO: 63 - DNA, 14 bp, Brassica napus
  source 1..14: mol_type = genomic DNA
aggagtcgttgttg

SEQ ID NO: 64 - DNA, 15 bp, Brassica napus
  source 1..15: mol_type = genomic DNA
ggaggaggaggagtc

SEQ ID NO: 65 - DNA, 15 bp, Brassica napus
  source 1..15: mol_type = genomic DNA
ggagtcgttgttgtt

SEQ ID NO: 66 - DNA, 15 bp, Brassica napus
  source 1..15: mol_type = genomic DNA
gagtcgttgttgttt

SEQ ID NO: 67 - DNA, 15 bp, Brassica napus
  source 1..15: mol_type = genomic DNA
agtcgttgttgtttt

SEQ ID NO: 68 - DNA, 15 bp, Brassica napus
  source 1..15: mol_type = genomic DNA
gtcgttgttgttttg

SEQ ID NO: 69 - DNA, 15 bp, Brassica napus
  source 1..15: mol_type = genomic DNA
tcgttgttgttttgc

SEQ ID NO: 70 - DNA, 15 bp, Brassica napus
  source 1..15: mol_type = genomic DNA
cgttgttgttttgcg

SEQ ID NO: 71 - DNA, 15 bp, Brassica napus
  source 1..15: mol_type = genomic DNA
gttgttgttttgcgg

SEQ ID NO: 72 - DNA, 15 bp, Brassica napus
  source 1..15: mol_type = genomic DNA
ttgttgttttgcggt

SEQ ID NO: 73 - DNA, 15 bp, Brassica napus
  source 1..15: mol_type = genomic DNA
tgttgttttgcggtg

SEQ ID NO: 74 - DNA, 15 bp, Brassica napus
  source 1..15: mol_type = genomic DNA
gttgttttgcggtgg

SEQ ID NO: 75 - DNA, 15 bp, Brassica napus
  source 1..15: mol_type = genomic DNA
gaggaggaggagtcg

SEQ ID NO: 76 - DNA, 15 bp, Brassica napus
  source 1..15: mol_type = genomic DNA
ttgttttgcggtggg

SEQ ID NO: 77 - DNA, 15 bp, Brassica napus
  source 1..15: mol_type = genomic DNA
aggaggaggagtcgt

SEQ ID NO: 78 - DNA, 15 bp, Brassica napus
  source 1..15: mol_type = genomic DNA
ggaggaggagtcgtt

SEQ ID NO: 79 - DNA, 15 bp, Brassica napus
  source 1..15: mol_type = genomic DNA
gaggaggagtcgttg

SEQ ID NO: 80 - DNA, 15 bp, Brassica napus
  source 1..15: mol_type = genomic DNA
aggaggagtcgttgt

SEQ ID NO: 81 - DNA, 15 bp, Brassica napus
  source 1..15: mol_type = genomic DNA
ggaggagtcgttgtt

SEQ ID NO: 82 - DNA, 15 bp, Brassica napus
  source 1..15: mol_type = genomic DNA
gaggagtcgttgttg

SEQ ID NO: 83 - DNA, 15 bp, Brassica napus
  source 1..15: mol_type = genomic DNA
aggagtcgttgttgt

SEQ ID NO: 84 - DNA, 16 bp, Brassica napus
  source 1..16: mol_type = genomic DNA
ggaggaggaggagtcg

SEQ ID NO: 85 - DNA, 16 bp, Brassica napus
  source 1..16: mol_type = genomic DNA
ggagtcgttgttgttt

SEQ ID NO: 86 - DNA, 16 bp, Brassica napus
  source 1..16: mol_type = genomic DNA
gagtcgttgttgtttt

SEQ ID NO: 87 - DNA, 16 bp, Brassica napus
  source 1..16: mol_type = genomic DNA
agtcgttgttgttttg

SEQ ID NO: 88 - DNA, 16 bp, Brassica napus
  source 1..16: mol_type = genomic DNA
gtcgttgttgttttgc

SEQ ID NO: 89 - DNA, 16 bp, Brassica napus
  source 1..16: mol_type = genomic DNA
tcgttgttgttttgcg

SEQ ID NO: 90 - DNA, 16 bp, Brassica napus
  source 1..16: mol_type = genomic DNA
cgttgttgttttgcgg

SEQ ID NO: 91 - DNA, 16 bp, Brassica napus
  source 1..16: mol_type = genomic DNA
gttgttgttttgcggt

SEQ ID NO: 92 - DNA, 16 bp, Brassica napus
  source 1..16: mol_type = genomic DNA
ttgttgttttgcggtg

SEQ ID NO: 93 - DNA, 16 bp, Brassica napus
  source 1..16: mol_type = genomic DNA
tgttgttttgcggtgg

SEQ ID NO: 94 - DNA, 16 bp, Brassica napus
  source 1..16: mol_type = genomic DNA
gttgttttgcggtggg

SEQ ID NO: 95 - DNA, 16 bp, Brassica napus
  source 1..16: mol_type = genomic DNA
gaggaggaggagtcgt

SEQ ID NO: 96 - DNA, 16 bp, Brassica napus
  source 1..16: mol_type = genomic DNA
aggaggaggagtcgtt

SEQ ID NO: 97 - DNA, 16 bp, Brassica napus
  source 1..16: mol_type = genomic DNA
ggaggaggagtcgttg

SEQ ID NO: 98 - DNA, 16 bp, Brassica napus
  source 1..16: mol_type = genomic DNA
gaggaggagtcgttgt

SEQ ID NO: 99 - DNA, 16 bp, Brassica napus
  source 1..16: mol_type = genomic DNA
aggaggagtcgttgtt

SEQ ID NO: 100 - DNA, 16 bp, Brassica napus
  source 1..16: mol_type = genomic DNA
ggaggagtcgttgttg

SEQ ID NO: 101 - DNA, 16 bp, Brassica napus
  source 1..16: mol_type = genomic DNA
gaggagtcgttgttgt

SEQ ID NO: 102 - DNA, 16 bp, Brassica napus
  source 1..16: mol_type = genomic DNA
aggagtcgttgttgtt

SEQ ID NO: 103 - DNA, 17 bp, Brassica napus
  source 1..17: mol_type = genomic DNA
ggaggaggaggagtcgt

SEQ ID NO: 104 - DNA, 17 bp, Brassica napus
  source 1..17: mol_type = genomic DNA
ggagtcgttgttgtttt

SEQ ID NO: 105 - DNA, 17 bp, Brassica napus
  source 1..17: mol_type = genomic DNA
gagtcgttgttgttttg

SEQ ID NO: 106 - DNA, 17 bp, Brassica napus
  source 1..17: mol_type = genomic DNA
agtcgttgttgttttgc

SEQ ID NO: 107 - DNA, 17 bp, Brassica napus
  source 1..17: mol_type = genomic DNA
gtcgttgttgttttgcg

SEQ ID NO: 108 - DNA, 17 bp, Brassica napus
  source 1..17: mol_type = genomic DNA
tcgttgttgttttgcgg

SEQ ID NO: 109 - DNA, 17 bp, Brassica napus
  source 1..17: mol_type = genomic DNA
cgttgttgttttgcggt

SEQ ID NO: 110 - DNA, 17 bp, Brassica napus
  source 1..17: mol_type = genomic DNA
gttgttgttttgcggtg

SEQ ID NO: 111 - DNA, 17 bp, Brassica napus
  source 1..17: mol_type = genomic DNA
ttgttgttttgcggtgg

SEQ ID NO: 112 - DNA, 17 bp, Brassica napus
  source 1..17: mol_type = genomic DNA
tgttgttttgcggtggg

SEQ ID NO: 113 - DNA, 17 bp, Brassica napus
  source 1..17: mol_type = genomic DNA
gaggaggaggagtcgtt

SEQ ID NO: 114 - DNA, 17 bp, Brassica napus
  source 1..17: mol_type = genomic DNA
aggaggaggagtcgttg

SEQ ID NO: 115 - DNA, 17 bp, Brassica napus
  source 1..17: mol_type = genomic DNA
ggaggaggagtcgttgt

SEQ ID NO: 116 - DNA, 17 bp, Brassica napus
  source 1..17: mol_type = genomic DNA
gaggaggagtcgttgtt

SEQ ID NO: 117 - DNA, 17 bp, Brassica napus
  source 1..17: mol_type = genomic DNA
aggaggagtcgttgttg

SEQ ID NO: 118 - DNA, 17 bp, Brassica napus
  source 1..17: mol_type = genomic DNA
ggaggagtcgttgttgt

SEQ ID NO: 119 - DNA, 17 bp, Brassica napus
  source 1..17: mol_type = genomic DNA
gaggagtcgttgttgtt

SEQ ID NO: 120 - DNA, 17 bp, Brassica napus
  source 1..17: mol_type = genomic DNA
aggagtcgttgttgttt

SEQ ID NO: 121 - DNA, 18 bp, Brassica napus
  source 1..18: mol_type = genomic DNA
ggaggaggaggagtcgtt

SEQ ID NO: 122 - DNA, 18 bp, Brassica napus
  source 1..18: mol_type = genomic DNA
ggagtcgttgttgttttg

SEQ ID NO: 123 - DNA, 18 bp, Brassica napus
  source 1..18: mol_type = genomic DNA
gagtcgttgttgttttgc

SEQ ID NO: 124 - DNA, 18 bp, Brassica napus
  source 1..18: mol_type = genomic DNA
agtcgttgttgttttgcg

SEQ ID NO: 125 - DNA, 18 bp, Brassica napus
  source 1..18: mol_type = genomic DNA
tcgttgttgttttgcggt

SEQ ID NO: 126 - DNA, 18 bp, Brassica napus
  source 1..18: mol_type = genomic DNA
cgttgttgttttgcggtg

SEQ ID NO: 127 - DNA, 18 bp, Brassica napus
  source 1..18: mol_type = genomic DNA
gttgttgttttgcggtgg

SEQ ID NO: 128 - DNA, 18 bp, Brassica napus
  source 1..18: mol_type = genomic DNA
ttgttgttttgcggtggg

SEQ ID NO: 129 - DNA, 18 bp, Brassica napus
  source 1..18: mol_type = genomic DNA
gtcgttgttgttttgcgg

SEQ ID NO: 130 - DNA, 18 bp, Brassica napus
  source 1..18: mol_type = genomic DNA
gaggaggaggagtcgttg

SEQ ID NO: 131 - DNA, 18 bp, Brassica napus
  source 1..18: mol_type = genomic DNA
aggaggaggagtcgttgt

SEQ ID NO: 132 - DNA, 18 bp, Brassica napus
  source 1..18: mol_type = genomic DNA
ggaggaggagtcgttgtt

SEQ ID NO: 133 - DNA, 18 bp, Brassica napus
  source 1..18: mol_type = genomic DNA
gaggaggagtcgttgttg

SEQ ID NO: 134 - DNA, 18 bp, Brassica napus
  source 1..18: mol_type = genomic DNA
aggaggagtcgttgttgt

SEQ ID NO: 135 - DNA, 18 bp, Brassica napus
  source 1..18: mol_type = genomic DNA
ggaggagtcgttgttgtt

SEQ ID NO: 136 - DNA, 18 bp, Brassica napus
  source 1..18: mol_type = genomic DNA
gaggagtcgttgttgttt

SEQ ID NO: 137 - DNA, 18 bp, Brassica napus
  source 1..18: mol_type = genomic DNA
aggagtcgttgttgtttt

SEQ ID NO: 138 - DNA, 13 bp, Brassica napus
  source 1..13: mol_type = genomic DNA
caccttactttgt

SEQ ID NO: 139 - DNA, 13 bp, Brassica napus
  source 1..13: mol_type = genomic DNA
ttgttaaatttaa

SEQ ID NO: 140 - DNA, 13 bp, Brassica napus
  source 1..13: mol_type = genomic DNA
tgttaaatttaaa

SEQ ID NO: 141 - DNA, 13 bp, Brassica napus
  source 1..13: mol_type = genomic DNA
gttaaatttaaaa

SEQ ID NO: 142 - DNA, 13 bp, Brassica napus
  source 1..13: mol_type = genomic DNA
ttaaatttaaaat

SEQ ID NO: 143 - DNA, 13 bp, Brassica napus
  source 1..13: mol_type = genomic DNA
taaatttaaaata

SEQ ID NO: 144 - DNA, 13 bp, Brassica napus
  source 1..13: mol_type = genomic DNA
aaatttaaaatag

SEQ ID NO: 145 - DNA, 13 bp, Brassica napus
  source 1..13: mol_type = genomic DNA
aatttaaaatagt

SEQ ID NO: 146 - DNA, 13 bp, Brassica napus
  source 1..13: mol_type = genomic DNA
atttaaaatagtt

SEQ ID NO: 147 - DNA, 13 bp, Brassica napus
  source 1..13: mol_type = genomic DNA
tttaaaatagtta

SEQ ID NO: 148 - DNA, 13 bp, Brassica napus
  source 1..13: mol_type = genomic DNA
ttaaaatagttac

SEQ ID NO: 149 - DNA, 13 bp, Brassica napus
  source 1..13: mol_type = genomic DNA
accttactttgtt

SEQ ID NO: 150 - DNA, 13 bp, Brassica napus
  source 1..13: mol_type = genomic DNA
taaaatagttaca

SEQ ID NO: 151 - DNA, 13 bp, Brassica napus
  source 1..13: mol_type = genomic DNA
aaaatagttacaa

SEQ ID NO: 152 - DNA, 13 bp, Brassica napus
  source 1..13: mol_type = genomic DNA
aaatagttacaaa

SEQ ID NO: 153 - DNA, 13 bp, Brassica napus
  source 1..13: mol_type = genomic DNA
ccttactttgtta

SEQ ID NO: 154 - DNA, 13 bp, Brassica napus
  source 1..13: mol_type = genomic DNA
cttactttgttaa

SEQ ID NO: 155 - DNA, 13 bp, Brassica napus
  source 1..13: mol_type = genomic DNA
ttactttgttaaa

SEQ ID NO: 156 - DNA, 13 bp, Brassica napus
  source 1..13: mol_type = genomic DNA
tactttgttaaat

SEQ ID NO: 157 - DNA, 13 bp, Brassica napus
  source 1..13: mol_type = genomic DNA
actttgttaaatt

SEQ ID NO: 158 - DNA, 13 bp, Brassica napus
  source 1..13: mol_type = genomic DNA
ctttgttaaattt

SEQ ID NO: 159 - DNA, 13 bp, Brassica napus
  source 1..13: mol_type = genomic DNA
tttgttaaattta

SEQ ID NO: 160 - DNA, 14 bp, Brassica napus
  source 1..14: mol_type = genomic DNA
caccttactttgtt

SEQ ID NO: 161 - DNA, 14 bp, Brassica napus
  source 1..14: mol_type = genomic DNA
ttgttaaatttaaa

SEQ ID NO: 162 - DNA, 14 bp, Brassica napus
  source 1..14: mol_type = genomic DNA
tgttaaatttaaaa

SEQ ID NO: 163 - DNA, 14 bp, Brassica napus
  source 1..14: mol_type = genomic DNA
gttaaatttaaaat

SEQ ID NO: 164 - DNA, 14 bp, Brassica napus
  source 1..14: mol_type = genomic DNA
ttaaatttaaaata

SEQ ID NO: 165 - DNA, 14 bp, Brassica napus
  source 1..14: mol_type = genomic DNA
taaatttaaaatag

SEQ ID NO: 166 - DNA, 14 bp, Brassica napus
  source 1..14: mol_type = genomic DNA
aaatttaaaatagt

SEQ ID NO: 167 - DNA, 14 bp, Brassica napus
  source 1..14: mol_type = genomic DNA
aatttaaaatagtt

SEQ ID NO: 168 - DNA, 14 bp, Brassica napus
  source 1..14: mol_type = genomic DNA
atttaaaatagtta

SEQ ID NO: 169 - DNA, 14 bp, Brassica napus
  source 1..14: mol_type = genomic DNA
tttaaaatagttac

SEQ ID NO: 170 - DNA, 14 bp, Brassica napus
  source 1..14: mol_type = genomic DNA
ttaaaatagttaca

SEQ ID NO: 171 - DNA, 14 bp, Brassica napus
  source 1..14: mol_type = genomic DNA
accttactttgtta

SEQ ID NO: 172 - DNA, 14 bp, Brassica napus
  source 1..14: mol_type = genomic DNA
taaaatagttacaa

SEQ ID NO: 173 - DNA, 14 bp, Brassica napus
  source 1..14: mol_type = genomic DNA
aaaatagttacaaa

SEQ ID NO: 174 - DNA, 14 bp, Brassica napus
  source 1..14: mol_type = genomic DNA
ccttactttgttaa

SEQ ID NO: 175 - DNA, 14 bp, Brassica napus
  source 1..14: mol_type = genomic DNA
cttactttgttaaa

SEQ ID NO: 176 - DNA, 14 bp, Brassica napus
  source 1..14: mol_type = genomic DNA
ttactttgttaaat

SEQ ID NO: 177 - DNA, 14 bp, Brassica napus
  source 1..14: mol_type = genomic DNA
tactttgttaaatt

SEQ ID NO: 178 - DNA, 14 bp, Brassica napus
  source 1..14: mol_type = genomic DNA
actttgttaaattt

SEQ ID NO: 179 - DNA, 14 bp, Brassica napus
  source 1..14: mol_type = genomic DNA
ctttgttaaattta

SEQ ID NO: 180 - DNA, 14 bp, Brassica napus
  source 1..14: mol_type = genomic DNA
tttgttaaatttaa

SEQ ID NO: 181 - DNA, 15 bp, Brassica napus
  source 1..15: mol_type = genomic DNA
caccttactttgtta

SEQ ID NO: 182 - DNA, 15 bp, Brassica napus
  source 1..15: mol_type = genomic DNA
ttgttaaatttaaaa

SEQ ID NO: 183 - DNA, 15 bp, Brassica napus
  source 1..15: mol_type = genomic DNA
tgttaaatttaaaat

SEQ ID NO: 184 - DNA, 15 bp, Brassica napus
  source 1..15: mol_type = genomic DNA
gttaaatttaaaata

SEQ ID NO: 185 - DNA, 15 bp, Brassica napus
  source 1..15: mol_type = genomic DNA
ttaaatttaaaatag

SEQ ID NO: 186 - DNA, 15 bp, Brassica napus
  source 1..15: mol_type = genomic DNA
taaatttaaaatagt

SEQ ID NO: 187 - DNA, 15 bp, Brassica napus
  source 1..15: mol_type = genomic DNA
aaatttaaaatagtt

SEQ ID NO: 188 - DNA, 15 bp, Brassica napus
  source 1..15: mol_type = genomic DNA
aatttaaaatagtta

SEQ ID NO: 189 - DNA, 15 bp, Brassica napus
  source 1..15: mol_type = genomic DNA
atttaaaatagttac

SEQ ID NO: 190 - DNA, 15 bp, Brassica napus
  source 1..15: mol_type = genomic DNA
tttaaaatagttaca

SEQ ID NO: 191 - DNA, 15 bp, Brassica napus
  source 1..15: mol_type = genomic DNA
ttaaaatagttacaa

SEQ ID NO: 192 - DNA, 15 bp, Brassica napus
  source 1..15: mol_type = genomic DNA
accttactttgttaa

SEQ ID NO: 193 - DNA, 15 bp, Brassica napus
  source 1..15: mol_type = genomic DNA
taaaatagttacaaa

SEQ ID NO: 194 - DNA, 15 bp, Brassica napus
  source 1..15: mol_type = genomic DNA
ccttactttgttaaa

SEQ ID NO: 195 - DNA, 15 bp, Brassica napus
  source 1..15: mol_type = genomic DNA
cttactttgttaaat

SEQ ID NO: 196 - DNA, 15 bp, Brassica napus
  source 1..15: mol_type = genomic DNA
ttactttgttaaatt

SEQ ID NO: 197 - DNA, 15 bp, Brassica napus
  source 1..15: mol_type = genomic DNA
tactttgttaaattt

SEQ ID NO: 198 - DNA, 15 bp, Brassica napus
  source 1..15: mol_type = genomic DNA
actttgttaaattta

SEQ ID NO: 199 - DNA, 15 bp, Brassica napus
  source 1..15: mol_type = genomic DNA
ctttgttaaatttaa

SEQ ID NO: 200 - DNA, 15 bp, Brassica napus
  source 1..15: mol_type = genomic DNA
tttgttaaatttaaa

SEQ ID NO: 201 - DNA, 16 bp, Brassica napus
  source 1..16: mol_type = genomic DNA
caccttactttgttaa

SEQ ID NO: 202 - DNA, 16 bp, Brassica napus
  source 1..16: mol_type = genomic DNA
ttgttaaatttaaaat

SEQ ID NO: 203 - DNA, 16 bp, Brassica napus
  source 1..16: mol_type = genomic DNA
tgttaaatttaaaata

SEQ ID NO: 204 - DNA, 16 bp, Brassica napus
  source 1..16: mol_type = genomic DNA
gttaaatttaaaatag

SEQ ID NO: 205 - DNA, 16 bp, Brassica napus
  source 1..16: mol_type = genomic DNA
ttaaatttaaaatagt

SEQ ID NO: 206 - DNA, 16 bp, Brassica napus
  source 1..16: mol_type = genomic DNA
taaatttaaaatagtt

SEQ ID NO: 207 - DNA, 16 bp, Brassica napus
  source 1..16: mol_type = genomic DNA
aaatttaaaatagtta

SEQ ID NO: 208 - DNA, 16 bp, Brassica napus
  source 1..16: mol_type = genomic DNA
aatttaaaatagttac

SEQ ID NO: 209 - DNA, 16 bp, Brassica napus
  source 1..16: mol_type = genomic DNA
atttaaaatagttaca

SEQ ID NO: 210 - DNA, 16 bp, Brassica napus
  source 1..16: mol_type = genomic DNA
tttaaaatagttacaa

SEQ ID NO: 211 - DNA, 16 bp, Brassica napus
  source 1..16: mol_type = genomic DNA
ttaaaatagttacaaa

SEQ ID NO: 212 - DNA, 16 bp, Brassica napus
  source 1..16: mol_type = genomic DNA
accttactttgttaaa

SEQ ID NO: 213 - DNA, 16 bp, Brassica napus
  source 1..16: mol_type = genomic DNA
ccttactttgttaaat

SEQ ID NO: 214 - DNA, 16 bp, Brassica napus
  source 1..16: mol_type = genomic DNA
cttactttgttaaatt

SEQ ID NO: 215 - DNA, 16 bp, Brassica napus
  source 1..16: mol_type = genomic DNA
ttactttgttaaattt

SEQ ID NO: 216 - DNA, 16 bp, Brassica napus
  source 1..16: mol_type = genomic DNA
tactttgttaaattta

SEQ ID NO: 217 - DNA, 16 bp, Brassica napus
  source 1..16: mol_type = genomic DNA
actttgttaaatttaa

SEQ ID NO: 218 - DNA, 16 bp, Brassica napus
  source 1..16: mol_type = genomic DNA
ctttgttaaatttaaa

SEQ ID NO: 219 - DNA, 16 bp, Brassica napus
  source 1..16: mol_type = genomic DNA
tttgttaaatttaaaa

SEQ ID NO: 220 - DNA, 17 bp, Brassica napus
  source 1..17: mol_type = genomic DNA
caccttactttgttaaa

SEQ ID NO: 221 - DNA, 17 bp, Brassica napus
  source 1..17: mol_type = genomic DNA
ttgttaaatttaaaata

SEQ ID NO: 222 - DNA, 17 bp, Brassica napus
  source 1..17: mol_type = genomic DNA
tgttaaatttaaaatag

SEQ ID NO: 223 - DNA, 17 bp, Brassica napus
  source 1..17: mol_type = genomic DNA
gttaaatttaaaatagt

SEQ ID NO: 224 - DNA, 17 bp, Brassica napus
  source 1..17: mol_type = genomic DNA
ttaaatttaaaatagtt

SEQ ID NO: 225 - DNA, 17 bp, Brassica napus
  source 1..17: mol_type = genomic DNA
taaatttaaaatagtta

SEQ ID NO: 226 - DNA, 17 bp, Brassica napus
  source 1..17: mol_type = genomic DNA
aaatttaaaatagttac

SEQ ID NO: 227 - DNA, 17 bp, Brassica napus
  source 1..17: mol_type = genomic DNA
aatttaaaatagttaca

SEQ ID NO: 228 - DNA, 17 bp, Brassica napus
  source 1..17: mol_type = genomic DNA
atttaaaatagttacaa

SEQ ID NO: 229 - DNA, 17 bp, Brassica napus
  source 1..17: mol_type = genomic DNA
tttaaaatagttacaaa

SEQ ID NO: 230 - DNA, 17 bp, Brassica napus
  source 1..17: mol_type = genomic DNA
accttactttgttaaat

SEQ ID NO: 231 - DNA, 17 bp, Brassica napus
  source 1..17: mol_type = genomic DNA
ccttactttgttaaatt

SEQ ID NO: 232 - DNA, 17 bp, Brassica napus
  source 1..17: mol_type = genomic DNA
cttactttgttaaattt

SEQ ID NO: 233 - DNA, 17 bp, Brassica napus
  source 1..17: mol_type = genomic DNA
ttactttgttaaattta

SEQ ID NO: 234 - DNA, 17 bp, Brassica napus
  source 1..17: mol_type = genomic DNA
tactttgttaaatttaa

SEQ ID NO: 235 - DNA, 17 bp, Brassica napus
  source 1..17: mol_type = genomic DNA
actttgttaaatttaaa

SEQ ID NO: 236 - DNA, 17 bp, Brassica napus
  source 1..17: mol_type = genomic DNA
ctttgttaaatttaaaa

SEQ ID NO: 237 - DNA, 17 bp, Brassica napus
  source 1..17: mol_type = genomic DNA
tttgttaaatttaaaat

SEQ ID NO: 238 - DNA, 18 bp, Brassica napus
  source 1..18: mol_type = genomic DNA
caccttactttgttaaat

SEQ ID NO: 239 - DNA, 18 bp, Brassica napus
  source 1..18: mol_type = genomic DNA
ttgttaaatttaaaatag

SEQ ID NO: 240 - DNA, 18 bp, Brassica napus
  source 1..18: mol_type = genomic DNA
tgttaaatttaaaatagt

SEQ ID NO: 241 - DNA, 18 bp, Brassica napus
  source 1..18: mol_type = genomic DNA
gttaaatttaaaatagtt

SEQ ID NO: 242 - DNA, 18 bp, Brassica napus
  source 1..18: mol_type = genomic DNA
taaatttaaaatagttac

SEQ ID NO: 243 - DNA, 18 bp, Brassica napus
  source 1..18: mol_type = genomic DNA
aaatttaaaatagttaca

SEQ ID NO: 244 - DNA, 18 bp, Brassica napus
  source 1..18: mol_type = genomic DNA
aatttaaaatagttacaa

SEQ ID NO: 245 - DNA, 18 bp, Brassica napus
  source 1..18: mol_type = genomic DNA
atttaaaatagttacaaa

SEQ ID NO: 246 - DNA, 18 bp, Brassica napus
  source 1..18: mol_type = genomic DNA
ttaaatttaaaatagtta

SEQ ID NO: 247 - DNA, 18 bp, Brassica napus
  source 1..18: mol_type = genomic DNA
accttactttgttaaatt

SEQ ID NO: 248 - DNA, 18 bp, Brassica napus
  source 1..18: mol_type = genomic DNA
ccttactttgttaaattt

SEQ ID NO: 249 - DNA, 18 bp, Brassica napus
  source 1..18: mol_type = genomic DNA
cttactttgttaaattta

SEQ ID NO: 250 - DNA, 18 bp, Brassica napus
  source 1..18: mol_type = genomic DNA
ttactttgttaaatttaa

SEQ ID NO: 251 - DNA, 18 bp, Brassica napus
  source 1..18: mol_type = genomic DNA
tactttgttaaatttaaa

SEQ ID NO: 252 - DNA, 18 bp, Brassica napus
  source 1..18: mol_type = genomic DNA
actttgttaaatttaaaa

SEQ ID NO: 253 - DNA, 18 bp, Brassica napus
  source 1..18: mol_type = genomic DNA
ctttgttaaatttaaaat

SEQ ID NO: 254 - DNA, 18 bp, Brassica napus
  source 1..18: mol_type = genomic DNA
tttgttaaatttaaaata

SEQ ID NO: 255 - DNA, 13 bp, Brassica napus
  source 1..13: mol_type = genomic DNA
gcaaagcaacgat

SEQ ID NO: 256 - DNA, 13 bp, Brassica napus
  source 1..13: mol_type = genomic DNA
cgattccgacgac

SEQ ID NO: 257 - DNA, 13 bp, Brassica napus
  source 1..13: mol_type = genomic DNA
gattccgacgact

SEQ ID NO: 258 - DNA, 13 bp, Brassica napus
  source 1..13: mol_type = genomic DNA
attccgacgactc

SEQ ID NO: 259 - DNA, 13 bp, Brassica napus
  source 1..13: mol_type = genomic DNA
ttccgacgactcc

SEQ ID NO: 260 - DNA, 13 bp, Brassica napus
  source 1..13: mol_type = genomic DNA
tccgacgactcct

SEQ ID NO: 261 - DNA, 13 bp, Brassica napus
  source 1..13: mol_type = genomic DNA
ccgacgactcctc

SEQ ID NO: 262 - DNA, 13 bp, Brassica napus
  source 1..13: mol_type = genomic DNA
cgacgactcctcc

SEQ ID NO: 263 - DNA, 13 bp, Brassica napus
  source 1..13: mol_type = genomic DNA
gacgactcctcct

SEQ ID NO: 264 - DNA, 13 bp, Brassica napus
  source 1..13: mol_type = genomic DNA
acgactcctcctc

SEQ ID NO: 265 - DNA, 13 bp, Brassica napus
  source 1..13: mol_type = genomic DNA
cgactcctcctcc

SEQ ID NO: 266 - DNA, 13 bp, Brassica napus
  source 1..13: mol_type = genomic DNA
caaagcaacgatt

SEQ ID NO: 267 - DNA, 13 bp, Brassica napus
  source 1..13: mol_type = genomic DNA
gactcctcctccg

SEQ ID NO: 268 - DNA, 13 bp, Brassica napus
  source 1..13: mol_type = genomic DNA
actcctcctccgc

SEQ ID NO: 269 - DNA, 13 bp, Brassica napus
  source 1..13: mol_type = genomic DNA
ctcctcctccgcc

SEQ ID NO: 270 - DNA, 13 bp, Brassica napus
  source 1..13: mol_type = genomic DNA
aaagcaacgattc

SEQ ID NO: 271 - DNA, 13 bp, Brassica napus
  source 1..13: mol_type = genomic DNA
aagcaacgattcc

SEQ ID NO: 272 - DNA, 13 bp, Brassica napus
  source 1..13: mol_type = genomic DNA
agcaacgattccg

SEQ ID NO: 273 - DNA, 13 bp, Brassica napus
  source 1..13: mol_type = genomic DNA
gcaacgattccga

SEQ ID NO: 274 - DNA, 13 bp, Brassica napus
  source 1..13: mol_type = genomic DNA
caacgattccgac

SEQ ID NO: 275 - DNA, 13 bp, Brassica napus
  source 1..13: mol_type = genomic DNA
aacgattccgacg

SEQ ID NO: 276 - DNA, 13 bp, Brassica napus
  source 1..13: mol_type = genomic DNA
acgattccgacga

SEQ ID NO: 277 - DNA, 14 bp, Brassica napus
  source 1..14: mol_type = genomic DNA
gcaaagcaacgatt

SEQ ID NO: 278 - DNA, 14 bp, Brassica napus
  source 1..14: mol_type = genomic DNA
cgattccgacgact

SEQ ID NO: 279 - DNA, 14 bp, Brassica napus
  source 1..14: mol_type = genomic DNA
gattccgacgactc

SEQ ID NO: 280 - DNA, 14 bp, Brassica napus
  source 1..14: mol_type = genomic DNA
attccgacgactcc

SEQ ID NO: 281 - DNA, 14 bp, Brassica napus
  source 1..14: mol_type = genomic DNA
ttccgacgactcct

SEQ ID NO: 282 - DNA, 14 bp, Brassica napus
  source 1..14: mol_type = genomic DNA
tccgacgactcctc

SEQ ID NO: 283 - DNA, 14 bp, Brassica napus
  source 1..14: mol_type = genomic DNA
ccgacgactcctcc

SEQ ID NO: 284 - DNA, 14 bp, Brassica napus
  source 1..14: mol_type = genomic DNA
cgacgactcctcct

SEQ ID NO: 285 - DNA, 14 bp, Brassica napus
  source 1..14: mol_type = genomic DNA
gacgactcctcctc

SEQ ID NO: 286 - DNA, 14 bp, Brassica napus
  source 1..14: mol_type = genomic DNA
acgactcctcctcc

SEQ ID NO: 287 - DNA, 14 bp, Brassica napus
  source 1..14: mol_type = genomic DNA
cgactcctcctccg

SEQ ID NO: 288 - DNA, 14 bp, Brassica napus
  source 1..14: mol_type = genomic DNA
caaagcaacgattc

SEQ ID NO: 289 - DNA, 14 bp, Brassica napus
  source 1..14: mol_type = genomic DNA
gactcctcctccgc

SEQ ID NO: 290 - DNA, 14 bp, Brassica napus
  source 1..14: mol_type = genomic DNA
actcctcctccgcc

SEQ ID NO: 291 - DNA, 14 bp, Brassica napus
  source 1..14: mol_type = genomic DNA
aaagcaacgattcc

SEQ ID NO: 292 - DNA, 14 bp, Brassica napus
  source 1..14: mol_type = genomic DNA
aagcaacgattccg

SEQ ID NO: 293 - DNA, 14 bp, Brassica napus
  source 1..14: mol_type = genomic DNA
agcaacgattccga

SEQ ID NO: 294 - DNA, 14 bp, Brassica napus
  source 1..14: mol_type = genomic DNA
gcaacgattccgac

SEQ ID NO: 295 - DNA, 14 bp, Brassica napus
  source 1..14: mol_type = genomic DNA
caacgattccgacg

SEQ ID NO: 296 - DNA, 14 bp, Brassica napus
  source 1..14: mol_type = genomic DNA
aacgattccgacga

SEQ ID NO: 297 - DNA, 14 bp, Brassica napus
  source 1..14: mol_type = genomic DNA
acgattccgacgac

SEQ ID NO: 298 - DNA, 15 bp, Brassica napus
  source 1..15: mol_type = genomic DNA
gcaaagcaacgattc

SEQ ID NO: 299 - DNA, 15 bp, Brassica napus
  source 1..15: mol_type = genomic DNA
cgattccgacgactc

SEQ ID NO: 300 - DNA, 15 bp, Brassica napus
  source 1..15: mol_type = genomic DNA
gattccgacgactcc

SEQ ID NO: 301 - DNA, 15 bp, Brassica napus
  source 1..15: mol_type = genomic DNA
attccgacgactcct

SEQ ID NO: 302 - DNA, 15 bp, Brassica napus
  source 1..15: mol_type = genomic DNA
ttccgacgactcctc

SEQ ID NO: 303 - DNA, 15 bp, Brassica napus
  source 1..15: mol_type = genomic DNA
tccgacgactcctcc

SEQ ID NO: 304 - DNA, 15 bp, Brassica napus
  source 1..15: mol_type = genomic DNA
ccgacgactcctcct

SEQ ID NO: 305 - DNA, 15 bp, Brassica napus
  source 1..15: mol_type = genomic DNA
cgacgactcctcctc

SEQ ID NO: 306 - DNA, 15 bp, Brassica napus
  source 1..15: mol_type = genomic DNA
gacgactcctcctcc

SEQ ID NO: 307 - DNA, 15 bp, Brassica napus
  source 1..15: mol_type = genomic DNA
acgactcctcctccg

SEQ ID NO: 308 - DNA, 15 bp, Brassica napus
  source 1..15: mol_type = genomic DNA
cgactcctcctccgc

SEQ ID NO: 309 - DNA, 15 bp, Brassica napus
  source 1..15: mol_type = genomic DNA
caaagcaacgattcc

SEQ ID NO: 310 - DNA, 15 bp, Brassica napus
  source 1..15: mol_type = genomic DNA
gactcctcctccgcc

SEQ ID NO: 311 - DNA, 15 bp, Brassica napus
  source 1..15: mol_type = genomic DNA
aaagcaacgattccg

SEQ ID NO: 312 - DNA, 15 bp, Brassica napus
  source 1..15: mol_type = genomic DNA
aagcaacgattccga

SEQ ID NO: 313 - DNA, 15 bp, Brassica napus
  source 1..15: mol_type = genomic DNA
agcaacgattccgac

SEQ ID NO: 314 - DNA, 15 bp, Brassica napus
  source 1..15: mol_type = genomic DNA
gcaacgattccgacg

SEQ ID NO: 315 - DNA, 15 bp, Brassica napus
  source 1..15: mol_type = genomic DNA
caacgattccgacga

SEQ ID NO: 316 - DNA, 15 bp, Brassica napus
  source 1..15: mol_type = genomic DNA
aacgattccgacgac

SEQ ID NO: 317 - DNA, 15 bp, Brassica napus
  source 1..15: mol_type = genomic DNA
acgattccgacgact

SEQ ID NO: 318 - DNA, 16 bp, Brassica napus
  source 1..16: mol_type = genomic DNA
gcaaagcaacgattcc

SEQ ID NO: 319 - DNA, 16 bp, Brassica napus
  source 1..16: mol_type = genomic DNA
cgattccgacgactcc

SEQ ID NO: 320 - DNA, 16 bp, Brassica napus
  source 1..16: mol_type = genomic DNA
gattccgacgactcct

SEQ ID NO: 321 - DNA, 16 bp, Brassica napus
  source 1..16: mol_type = genomic DNA
attccgacgactcctc

SEQ ID NO: 322 - DNA, 16 bp, Brassica napus
  source 1..16: mol_type = genomic DNA
ttccgacgactcctcc

SEQ ID NO: 323 - DNA, 16 bp, Brassica napus
  source 1..16: mol_type = genomic DNA
tccgacgactcctcct

SEQ ID NO: 324 - DNA, 16 bp, Brassica napus
  source 1..16: mol_type = genomic DNA
ccgacgactcctcctc

SEQ ID NO: 325 - DNA, 16 bp, Brassica napus
  source 1..16: mol_type = genomic DNA
cgacgactcctcctcc

SEQ ID NO: 326 - DNA, 16 bp, Brassica napus
  source 1..16: mol_type = genomic DNA
gacgactcctcctccg

SEQ ID NO: 327 - DNA, 16 bp, Brassica napus
  source 1..16: mol_type = genomic DNA
acgactcctcctccgc

SEQ ID NO: 328 - DNA, 16 bp, Brassica napus
  source 1..16: mol_type = genomic DNA
cgactcctcctccgcc

SEQ ID NO: 329 - DNA, 16 bp, Brassica napus
  source 1..16: mol_type = genomic DNA
caaagcaacgattccg

SEQ ID NO: 330 - DNA, 16 bp, Brassica napus
  source 1..16: mol_type = genomic DNA
aaagcaacgattccga

SEQ ID NO: 331 - DNA, 16 bp, Brassica napus
  source 1..16: mol_type = genomic DNA
aagcaacgattccgac

SEQ ID NO: 332 - DNA, 16 bp, Brassica napus
  source 1..16: mol_type = genomic DNA
agcaacgattccgacg

SEQ ID NO: 333 - DNA, 16 bp, Brassica napus
  source 1..16: mol_type = genomic DNA
gcaacgattccgacga

SEQ ID NO: 334 - DNA, 16 bp, Brassica napus
  source 1..16: mol_type = genomic DNA
caacgattccgacgac

SEQ ID NO: 335 - DNA, 16 bp, Brassica napus
  source 1..16: mol_type = genomic DNA
aacgattccgacgact

SEQ ID NO: 336 - DNA, 16 bp, Brassica napus
  source 1..16: mol_type = genomic DNA
acgattccgacgactc

SEQ ID NO: 337 - DNA, 17 bp, Brassica napus
  source 1..17: mol_type = genomic DNA
gcaaagcaacgattccg

SEQ ID NO: 338 - DNA, 17 bp, Brassica napus
  source 1..17: mol_type = genomic DNA
cgattccgacgactcct

SEQ ID NO: 339 - DNA, 17 bp, Brassica napus
  source 1..17: mol_type = genomic DNA
gattccgacgactcctc

SEQ ID NO: 340 - DNA, 17 bp, Brassica napus
  source 1..17: mol_type = genomic DNA
attccgacgactcctcc

SEQ ID NO: 341 - DNA, 17 bp, Brassica napus
  source 1..17: mol_type = genomic DNA
ttccgacgactcctcct

SEQ ID NO: 342 - DNA, 17 bp, Brassica napus
  source 1..17: mol_type = genomic DNA
tccgacgactcctcctc

SEQ ID NO: 343 - DNA, 17 bp, Brassica napus
  source 1..17: mol_type = genomic DNA
ccgacgactcctcctcc

SEQ ID NO: 344 - DNA, 17 bp, Brassica napus
  source 1..17: mol_type = genomic DNA
cgacgactcctcctccg

SEQ ID NO: 345 - DNA, 17 bp, Brassica napus
  source 1..17: mol_type = genomic DNA
gacgactcctcctccgc

SEQ ID NO: 346 - DNA, 17 bp, Brassica napus
  source 1..17: mol_type = genomic DNA
acgactcctcctccgcc

SEQ ID NO: 347 - DNA, 17 bp, Brassica napus
  source 1..17: mol_type = genomic DNA
caaagcaacgattccga

SEQ ID NO: 348 - DNA, 17 bp, Brassica napus
  source 1..17: mol_type = genomic DNA
aaagcaacgattccgac

SEQ ID NO: 349 - DNA, 17 bp, Brassica napus
  source 1..17: mol_type = genomic DNA
aagcaacgattccgacg

SEQ ID NO: 350 - DNA, 17 bp, Brassica napus
  source 1..17: mol_type = genomic DNA
agcaacgattccgacga

SEQ ID NO: 351 - DNA, 17 bp, Brassica napus
  source 1..17: mol_type = genomic DNA
gcaacgattccgacgac

SEQ ID NO: 352 - DNA, 17 bp, Brassica napus
  source 1..17: mol_type = genomic DNA
caacgattccgacgact

SEQ ID NO: 353 - DNA, 17 bp, Brassica napus
  source 1..17: mol_type = genomic DNA
aacgattccgacgactc

SEQ ID NO: 354 - DNA, 17 bp, Brassica napus
  source 1..17: mol_type = genomic DNA
acgattccgacgactcc

SEQ ID NO: 355 - DNA, 18 bp, Brassica napus
  source 1..18: mol_type = genomic DNA
gcaaagcaacgattccga

SEQ ID NO: 356 - DNA, 18 bp, Brassica napus
  source 1..18: mol_type = genomic DNA
cgattccgacgactcctc

SEQ ID NO: 357 - DNA, 18 bp, Brassica napus
  source 1..18: mol_type = genomic DNA
gattccgacgactcctcc

SEQ ID NO: 358 - DNA, 18 bp, Brassica napus
  source 1..18: mol_type = genomic DNA
attccgacgactcctcct

SEQ ID NO: 359 - DNA, 18 bp, Brassica napus
  source 1..18: mol_type = genomic DNA
tccgacgactcctcctcc

SEQ ID NO: 360 - DNA, 18 bp, Brassica napus
  source 1..18: mol_type = genomic DNA
ccgacgactcctcctccg

SEQ ID NO: 361 - DNA, 18 bp, Brassica napus
  source 1..18: mol_type = genomic DNA
cgacgactcctcctccgc

SEQ ID NO: 362 - DNA, 18 bp, Brassica napus
  source 1..18: mol_type = genomic DNA
gacgactcctcctccgcc

SEQ ID NO: 363 - DNA, 18 bp, Brassica napus
  source 1..18: mol_type = genomic DNA
ttccgacgactcctcctc

SEQ ID NO: 364 - DNA, 18 bp, Brassica napus
  source 1..18: mol_type = genomic DNA
caaagcaacgattccgac

SEQ ID NO: 365 - DNA, 18 bp, Brassica napus
  source 1..18: mol_type = genomic DNA
aaagcaacgattccgacg

SEQ ID NO: 366 - DNA, 18 bp, Brassica napus
  source 1..18: mol_type = genomic DNA
aagcaacgattccgacga

SEQ ID NO: 367 - DNA, 18 bp, Brassica napus
  source 1..18: mol_type = genomic DNA
agcaacgattccgacgac

SEQ ID NO: 368 - DNA, 18 bp, Brassica napus
  source 1..18: mol_type = genomic DNA
gcaacgattccgacgact

SEQ ID NO: 369 - DNA, 18 bp, Brassica napus
  source 1..18: mol_type = genomic DNA
caacgattccgacgactc

SEQ ID NO: 370 - DNA, 18 bp, Brassica napus
  source 1..18: mol_type = genomic DNA
aacgattccgacgactcc

SEQ ID NO: 371 - DNA, 18 bp, Brassica napus
  source 1..18: mol_type = genomic DNA
acgattccgacgactcct

SEQ ID NO: 372 - DNA, 13 bp, Brassica napus
  source 1..13: mol_type = genomic DNA
acaccctactttg

SEQ ID NO: 373 - DNA, 13 bp, Brassica napus
  source 1..13: mol_type = genomic DNA
tttgtgaaaagtc

SEQ ID NO: 374 - DNA, 13 bp, Brassica napus
  source 1..13: mol_type = genomic DNA
ttgtgaaaagtcg

SEQ ID NO: 375 - DNA, 13 bp, Brassica napus
  source 1..13: mol_type = genomic DNA
tgtgaaaagtcgc

SEQ ID NO: 376 - DNA, 13 bp, Brassica napus
  source 1..13: mol_type = genomic DNA
gtgaaaagtcgct

SEQ ID NO: 377 - DNA, 13 bp, Brassica napus
  source 1..13: mol_type = genomic DNA
tgaaaagtcgctg

SEQ ID NO: 378 - DNA, 13 bp, Brassica napus
  source 1..13: mol_type = genomic DNA
gaaaagtcgctga

SEQ ID NO: 379 - DNA, 13 bp, Brassica napus
  source 1..13: mol_type = genomic DNA
aaaagtcgctgat

SEQ ID NO: 380 - DNA, 13 bp, Brassica napus
  source 1..13: mol_type = genomic DNA
aaagtcgctgatc

SEQ ID NO: 381 - DNA, 13 bp, Brassica napus
  source 1..13: mol_type = genomic DNA
aagtcgctgatct

SEQ ID NO: 382 - DNA, 13 bp, Brassica napus
  source 1..13: mol_type = genomic DNA
agtcgctgatctt

SEQ ID NO: 383 - DNA, 13 bp, Brassica napus
  source 1..13: mol_type = genomic DNA
caccctactttgt

SEQ ID NO: 384 - DNA, 13 bp, Brassica napus
  source 1..13: mol_type = genomic DNA
gtcgctgatctta

SEQ ID NO: 385 - DNA, 13 bp, Brassica napus
  source 1..13: mol_type = genomic DNA
tcgctgatcttat

SEQ ID NO: 386 - DNA, 13 bp, Brassica napus
  source 1..13: mol_type = genomic DNA
cgctgatcttatt

SEQ ID NO: 387 - DNA, 13 bp, Brassica napus
  source 1..13: mol_type = genomic DNA
accctactttgtg

SEQ ID NO: 388 - DNA, 13 bp, Brassica napus
  source 1..13: mol_type = genomic DNA
ccctactttgtga

SEQ ID NO: 389 - DNA, 13 bp, Brassica napus
  source 1..13: mol_type = genomic DNA
cctactttgtgaa

SEQ ID NO: 390 - DNA, 13 bp, Brassica napus
  source 1..13: mol_type = genomic DNA
ctactttgtgaaa

SEQ ID NO: 391 - DNA, 13 bp, Brassica napus
  source 1..13: mol_type = genomic DNA
tactttgtgaaaa

SEQ ID NO: 392 - DNA, 13 bp, Brassica napus
  source 1..13: mol_type = genomic DNA
actttgtgaaaag

SEQ ID NO: 393 - DNA, 13 bp, Brassica napus
  source 1..13: mol_type = genomic DNA
ctttgtgaaaagt

SEQ ID NO: 394 - DNA, 14 bp, Brassica napus
  source 1..14: mol_type = genomic DNA
acaccctactttgt

SEQ ID NO: 395 - DNA, 14 bp, Brassica napus
  source 1..14: mol_type = genomic DNA
tttgtgaaaagtcg

SEQ ID NO: 396 - DNA, 14 bp, Brassica napus
  source 1..14: mol_type = genomic DNA
ttgtgaaaagtcgc

SEQ ID NO: 397 - DNA, 14 bp, Brassica napus
  source 1..14: mol_type = genomic DNA
tgtgaaaagtcgct

SEQ ID NO: 398 - DNA, 14 bp, Brassica napus
  source 1..14: mol_type = genomic DNA
gtgaaaagtcgctg

SEQ ID NO: 399 - DNA, 14 bp, Brassica napus
  source 1..14: mol_type = genomic DNA
tgaaaagtcgctga

SEQ ID NO: 400 - DNA, 14 bp, Brassica napus
  source 1..14: mol_type = genomic DNA
gaaaagtcgctgat

SEQ ID NO: 401 - DNA, 14 bp, Brassica napus
  source 1..14: mol_type = genomic DNA
aaaagtcgctgatc

SEQ ID NO: 402 - DNA, 14 bp, Brassica napus
  source 1..14: mol_type = genomic DNA
aaagtcgctgatct

SEQ ID NO: 403 - DNA, 14 bp, Brassica napus
  source 1..14: mol_type = genomic DNA
aagtcgctgatctt

SEQ ID NO: 404 - DNA, 14 bp, Brassica napus
  source 1..14: mol_type = genomic DNA
agtcgctgatctta

SEQ ID NO: 405 - DNA, 14 bp, Brassica napus
  source 1..14: mol_type = genomic DNA
caccctactttgtg

SEQ ID NO: 406 - DNA, 14 bp, Brassica napus
  source 1..14: mol_type = genomic DNA
gtcgctgatcttat

SEQ ID NO: 407 - DNA, 14 bp, Brassica napus
  source 1..14: mol_type = genomic DNA
tcgctgatcttatt

SEQ ID NO: 408 - DNA, 14 bp, Brassica napus
  source 1..14: mol_type = genomic DNA
accctactttgtga

SEQ ID NO: 409 - DNA, 14 bp, Brassica napus
  source 1..14: mol_type = genomic DNA
ccctactttgtgaa

SEQ ID NO: 410 - DNA, 14 bp, Brassica napus
  source 1..14: mol_type = genomic DNA
cctactttgtgaaa

SEQ ID NO: 411 - DNA, 14 bp, Brassica napus
  source 1..14: mol_type = genomic DNA
ctactttgtgaaaa

SEQ ID NO: 412 - DNA, 14 bp, Brassica napus
  source 1..14: mol_type = genomic DNA
tactttgtgaaaag

SEQ ID NO: 413 - DNA, 14 bp, Brassica napus
  source 1..14: mol_type = genomic DNA
actttgtgaaaagt

SEQ ID NO: 414 - DNA, 14 bp, Brassica napus
  source 1..14: mol_type = genomic DNA
ctttgtgaaaagtc

SEQ ID NO: 415 - DNA, 15 bp, Brassica napus
  source 1..15: mol_type = genomic DNA
acaccctactttgtg

SEQ ID NO: 416 - DNA, 15 bp, Brassica napus
  source 1..15: mol_type = genomic DNA
tttgtgaaaagtcgc

SEQ ID NO: 417 - DNA, 15 bp, Brassica napus
  source 1..15: mol_type = genomic DNA
ttgtgaaaagtcgct

SEQ ID NO: 418 - DNA, 15 bp, Brassica napus
  source 1..15: mol_type = genomic DNA
tgtgaaaagtcgctg

SEQ ID NO: 419 - DNA, 15 bp, Brassica napus
  source 1..15: mol_type = genomic DNA
gtgaaaagtcgctga

SEQ ID NO: 420 - DNA, 15 bp, Brassica napus
  source 1..15: mol_type = genomic DNA
tgaaaagtcgctgat

SEQ ID NO: 421 - DNA, 15 bp, Brassica napus
  source 1..15: mol_type = genomic DNA
gaaaagtcgctgatc

SEQ ID NO: 422 - DNA, 15 bp, Brassica napus
  source 1..15: mol_type = genomic DNA
aaaagtcgctgatct

SEQ ID NO: 423 - DNA, 15 bp, Brassica napus
  source 1..15: mol_type = genomic DNA
aaagtcgctgatctt

SEQ ID NO: 424 - DNA, 15 bp, Brassica napus
  source 1..15: mol_type = genomic DNA
aagtcgctgatctta

SEQ ID NO: 425 - DNA, 15 bp, Brassica napus
  source 1..15: mol_type = genomic DNA
agtcgctgatcttat

SEQ ID NO: 426 - DNA, 15 bp, Brassica napus
  source 1..15: mol_type = genomic DNA
caccctactttgtga

SEQ ID NO: 427 - DNA, 15 bp, Brassica napus
  source 1..15: mol_type = genomic DNA
gtcgctgatcttatt

SEQ ID NO: 428 - DNA, 15 bp, Brassica napus
  source 1..15: mol_type = genomic DNA
accctactttgtgaa

SEQ ID NO: 429 - DNA, 15 bp, Brassica napus
  source 1..15: mol_type = genomic DNA
ccctactttgtgaaa

SEQ ID NO: 430 - DNA, 15 bp, Brassica napus
  source 1..15: mol_type = genomic DNA
cctactttgtgaaaa

SEQ ID NO: 431 - DNA, 15 bp, Brassica napus
  source 1..15: mol_type = genomic DNA
ctactttgtgaaaag

SEQ ID NO: 432 - DNA, 15 bp, Brassica napus
  source 1..15: mol_type = genomic DNA
tactttgtgaaaagt

SEQ ID NO: 433 - DNA, 15 bp, Brassica napus
  source 1..15: mol_type = genomic DNA
actttgtgaaaagtc

SEQ ID NO: 434 - DNA, 15 bp, Brassica napus
  source 1..15: mol_type = genomic DNA
ctttgtgaaaagtcg

SEQ ID NO: 435 - DNA, 16 bp, Brassica napus
  source 1..16: mol_type = genomic DNA
acaccctactttgtga

SEQ ID NO: 436 - DNA, 16 bp, Brassica napus
  source 1..16: mol_type = genomic DNA
tttgtgaaaagtcgct

SEQ ID NO: 437 - DNA, 16 bp, Brassica napus
  source 1..16: mol_type = genomic DNA
ttgtgaaaagtcgctg

SEQ ID NO: 438 - DNA, 16 bp, Brassica napus
  source 1..16: mol_type = genomic DNA
tgtgaaaagtcgctga

SEQ ID NO: 439 - DNA, 16 bp, Brassica napus
  source 1..16: mol_type = genomic DNA
gtgaaaagtcgctgat

SEQ ID NO: 440 - DNA, 16 bp, Brassica napus
  source 1..16: mol_type = genomic DNA
tgaaaagtcgctgatc

SEQ ID NO: 441 - DNA, 16 bp, Brassica napus
  source 1..16: mol_type = genomic DNA
gaaaagtcgctgatct

SEQ ID NO: 442 - DNA, 16 bp, Brassica napus
  source 1..16: mol_type = genomic DNA
aaaagtcgctgatctt

SEQ ID NO: 443 - DNA, 16 bp, Brassica napus
  source 1..16: mol_type = genomic DNA
aaagtcgctgatctta

SEQ ID NO: 444 - DNA, 16 bp, Brassica napus
  source 1..16: mol_type = genomic DNA
aagtcgctgatcttat

SEQ ID NO: 445 - DNA, 16 bp, Brassica napus
  source 1..16: mol_type = genomic DNA
agtcgctgatcttatt

SEQ ID NO: 446 - DNA, 16 bp, Brassica napus
  source 1..16: mol_type = genomic DNA
caccctactttgtgaa

SEQ ID NO: 447 - DNA, 16 bp, Brassica napus
  source 1..16: mol_type = genomic DNA
accctactttgtgaaa

SEQ ID NO: 448 - DNA, 16 bp, Brassica napus
  source 1..16: mol_type = genomic DNA
ccctactttgtgaaaa

SEQ ID NO: 449 - DNA, 16 bp, Brassica napus
  source 1..16: mol_type = genomic DNA
cctactttgtgaaaag

SEQ ID NO: 450 - DNA, 16 bp, Brassica napus
  source 1..16: mol_type = genomic DNA
ctactttgtgaaaagt

SEQ ID NO: 451 - DNA, 16 bp, Brassica napus
  source 1..16: mol_type = genomic DNA
tactttgtgaaaagtc

SEQ ID NO: 452 - DNA, 16 bp, Brassica napus
  source 1..16: mol_type = genomic DNA
actttgtgaaaagtcg

SEQ ID NO: 453 - DNA, 16 bp, Brassica napus
  source 1..16: mol_type = genomic DNA
ctttgtgaaaagtcgc

SEQ ID NO: 454 - DNA, 17 bp, Brassica napus
  source 1..17: mol_type = genomic DNA
acaccctactttgtgaa

SEQ ID NO: 455 - DNA, 17 bp, Brassica napus
  source 1..17: mol_type = genomic DNA
tttgtgaaaagtcgctg

SEQ ID NO: 456 - DNA, 17 bp, Brassica napus
  source 1..17: mol_type = genomic DNA
ttgtgaaaagtcgctga

SEQ ID NO: 457 - DNA, 17 bp, Brassica napus
  source 1..17: mol_type = genomic DNA
tgtgaaaagtcgctgat

SEQ ID NO: 458 - DNA, 17 bp, Brassica napus
  source 1..17: mol_type = genomic DNA
gtgaaaagtcgctgatc

SEQ ID NO: 459 - DNA, 17 bp, Brassica napus
  source 1..17: mol_type = genomic DNA
tgaaaagtcgctgatct

SEQ ID NO: 460 - DNA, 17 bp, Brassica napus
  source 1..17: mol_type = genomic DNA
gaaaagtcgctgatctt

SEQ ID NO: 461 - DNA, 17 bp, Brassica napus
  source 1..17: mol_type = genomic DNA
aaaagtcgctgatctta

SEQ ID NO: 462 - DNA, 17 bp, Brassica napus
  source 1..17: mol_type = genomic DNA
aaagtcgctgatcttat

SEQ ID NO: 463 - DNA, 17 bp, Brassica napus
  source 1..17: mol_type = genomic DNA
aagtcgctgatcttatt

SEQ ID NO: 464 - DNA, 17 bp, Brassica napus
  source 1..17: mol_type = genomic DNA
caccctactttgtgaaa

SEQ ID NO: 465 - DNA, 17 bp, Brassica napus
  source 1..17: mol_type = genomic DNA
accctactttgtgaaaa

SEQ ID NO: 466 - DNA, 17 bp, Brassica napus
  source 1..17: mol_type = genomic DNA
ccctactttgtgaaaag

SEQ ID NO: 467 - DNA, 17 bp, Brassica napus
  source 1..17: mol_type = genomic DNA
cctactttgtgaaaagt

SEQ ID NO: 468 - DNA, 17 bp, Brassica napus
  source 1..17: mol_type = genomic DNA
ctactttgtgaaaagtc

SEQ ID NO: 469 - DNA, 17 bp, Brassica napus
  source 1..17: mol_type = genomic DNA
tactttgtgaaaagtcg

SEQ ID NO: 470 - DNA, 17 bp, Brassica napus
  source 1..17: mol_type = genomic DNA
actttgtgaaaagtcgc

SEQ ID NO: 471 - DNA, 17 bp, Brassica napus
  source 1..17: mol_type = genomic DNA
ctttgtgaaaagtcgct

SEQ ID NO: 472 - DNA, 18 bp, Brassica napus
  source 1..18: mol_type = genomic DNA
acaccctactttgtgaaa

SEQ ID NO: 473 - DNA, 18 bp, Brassica napus
  source 1..18: mol_type = genomic DNA
tttgtgaaaagtcgctga

SEQ ID NO: 474 - DNA, 18 bp, Brassica napus
  source 1..18: mol_type = genomic DNA
ttgtgaaaagtcgctgat

SEQ ID NO: 475 - DNA, 18 bp, Brassica napus
  source 1..18: mol_type = genomic DNA
tgtgaaaagtcgctgatc

SEQ ID NO: 476 - DNA, 18 bp, Brassica napus
  source 1..18: mol_type = genomic DNA
tgaaaagtcgctgatctt

SEQ ID NO: 477 - DNA, 18 bp, Brassica napus
  source 1..18: mol_type = genomic DNA
gaaaagtcgctgatctta

SEQ ID NO: 478 - DNA, 18 bp, Brassica napus
  source 1..18: mol_type = genomic DNA
aaaagtcgctgatcttat

SEQ ID NO: 479 - DNA, 18 bp, Brassica napus
  source 1..18: mol_type = genomic DNA
aaagtcgctgatcttatt

SEQ ID NO: 480 - DNA, 18 bp, Brassica napus
  source 1..18: mol_type = genomic DNA
gtgaaaagtcgctgatct

SEQ ID NO: 481 - DNA, 18 bp, Brassica napus
  source 1..18: mol_type = genomic DNA
caccctactttgtgaaaa

SEQ ID NO: 482 - DNA, 18 bp, Brassica napus
  source 1..18: mol_type = genomic DNA
accctactttgtgaaaag

SEQ ID NO: 483 - DNA, 18 bp, Brassica napus
  source 1..18: mol_type = genomic DNA
ccctactttgtgaaaagt

SEQ ID NO: 484 - DNA, 18 bp, Brassica napus
  source 1..18: mol_type = genomic DNA
cctactttgtgaaaagtc

SEQ ID NO: 485 - DNA, 18 bp, Brassica napus
  source 1..18: mol_type = genomic DNA
ctactttgtgaaaagtcg

SEQ ID NO: 486 - DNA, 18 bp, Brassica napus
  source 1..18: mol_type = genomic DNA
tactttgtgaaaagtcgc

SEQ ID NO: 487 - DNA, 18 bp, Brassica napus
  source 1..18: mol_type = genomic DNA
actttgtgaaaagtcgct

SEQ ID NO: 488 - DNA, 18 bp, Brassica napus
  source 1..18: mol_type = genomic DNA
ctttgtgaaaagtcgctg

SEQ ID NO: 489 - DNA, 15 bp, Brassica napus
  source 1..15: mol_type = genomic DNA
ttttaactatagtgg

SEQ ID NO: 490 - DNA, 16 bp, Brassica napus
  source 1..16: mol_type = genomic DNA
ttttaactatagtggc

SEQ ID NO: 491 - DNA, 17 bp, Brassica napus
  source 1..17: mol_type = genomic DNA
ttttaactatagtggcc

SEQ ID NO: 492 - DNA, 18 bp, Brassica napus
  source 1..18: mol_type = genomic DNA
ttttaactatagtggcct

SEQ ID NO: 493 - DNA, 19 bp, Brassica napus
  source 1..19: mol_type = genomic DNA
ttttaactatagtggcctt

SEQ ID NO: 494 - DNA, 20 bp, Brassica napus
  source 1..20: mol_type = genomic DNA
ttttaactatagtggccttc

SEQ ID NO: 495 - DNA, 21 bp, Brassica napus
  source 1..21: mol_type = genomic DNA
ttttaactatagtggccttca

SEQ ID NO: 496 - DNA, 22 bp, Brassica napus
  source 1..22: mol_type = genomic DNA
ttttaactatagtggccttcac

SEQ ID NO: 497 - DNA, 23 bp, Brassica napus
  source 1..23: mol_type = genomic DNA
ttttaactatagtggccttcacg

SEQ ID NO: 498 - DNA, 15 bp, Brassica napus
  source 1..15: mol_type = genomic DNA
tagtggccttcacgc

SEQ ID NO: 499 - DNA, 16 bp, Brassica napus
  source 1..16: mol_type = genomic DNA
tagtggccttcacgct

SEQ ID NO: 500 - DNA, 17 bp, Brassica napus
  source 1..17: mol_type = genomic DNA
tagtggccttcacgctc

SEQ ID NO: 501 - DNA, 18 bp, Brassica napus
  source 1..18: mol_type = genomic DNA
tagtggccttcacgctcc

SEQ ID NO: 502 - DNA, 19 bp, Brassica napus
  source 1..19: mol_type = genomic DNA
tagtggccttcacgctccg

SEQ ID NO: 503 - DNA, 20 bp, Brassica napus
  source 1..20: mol_type = genomic DNA
tagtggccttcacgctccga

SEQ ID NO: 504 - DNA, 21 bp, Brassica napus
  source 1..21: mol_type = genomic DNA
tagtggccttcacgctccgat

SEQ ID NO: 505 - DNA, 22 bp, Brassica napus
  source 1..22: mol_type = genomic DNA
tagtggccttcacgctccgatg

SEQ ID NO: 506 - DNA, 23 bp, Brassica napus
  source 1..23: mol_type = genomic DNA
tagtggccttcacgctccgatgt

SEQ ID NO: 507 - DNA, 15 bp, Brassica napus
  source 1..15: mol_type = genomic DNA
tgggtctttcaactg

SEQ ID NO: 508 - DNA, 16 bp, Brassica napus
  source 1..16: mol_type = genomic DNA
tgggtctttcaactga

SEQ ID NO: 509 - DNA, 17 bp, Brassica napus
  source 1..17: mol_type = genomic DNA
tgggtctttcaactgag

SEQ ID NO: 510 - DNA, 18 bp, Brassica napus
  source 1..18: mol_type = genomic DNA
tgggtctttcaactgagc

SEQ ID NO: 511 - DNA, 19 bp, Brassica napus
  source 1..19: mol_type = genomic DNA
tgggtctttcaactgagct

SEQ ID NO: 512 - DNA, 20 bp, Brassica napus
  source 1..20: mol_type = genomic DNA
tgggtctttcaactgagctc

SEQ ID NO: 513 - DNA, 21 bp, Brassica napus
  source 1..21: mol_type = genomic DNA
tgggtctttcaactgagctca

SEQ ID NO: 514 - DNA, 22 bp, Brassica napus
  source 1..22: mol_type = genomic DNA
tgggtctttcaactgagctcaa

SEQ ID NO: 515 - DNA, 23 bp, Brassica napus
  source 1..23: mol_type = genomic DNA
tgggtctttcaactgagctcaat

SEQ ID NO: 516 - DNA, 15 bp, Brassica napus
  source 1..15: mol_type = genomic DNA
gggtctttcaactga

SEQ ID NO: 517 - DNA, 16 bp, Brassica napus
  source 1..16: mol_type = genomic DNA
gggtctttcaactgag

SEQ ID NO: 518 - DNA, 17 bp, Brassica napus
  source 1..17: mol_type = genomic DNA
gggtctttcaactgagc

SEQ ID NO: 519 - DNA, 18 bp, Brassica napus
  source 1..18: mol_type = genomic DNA
gggtctttcaactgagct

SEQ ID NO: 520 - DNA, 19 bp, Brassica napus
  source 1..19: mol_type = genomic DNA
gggtctttcaactgagctc

SEQ ID NO: 521 - DNA, 20 bp, Brassica napus
  source 1..20: mol_type = genomic DNA
gggtctttcaactgagctca

SEQ ID NO: 522 - DNA, 21 bp, Brassica napus
  source 1..21: mol_type = genomic DNA
gggtctttcaactgagctcaa

SEQ ID NO: 523 - DNA, 22 bp, Brassica napus
  source 1..22: mol_type = genomic DNA
gggtctttcaactgagctcaat

SEQ ID NO: 524 - DNA, 23 bp, Brassica napus
  source 1..23: mol_type = genomic DNA
gggtctttcaactgagctcaata

SEQ ID NO: 525 - DNA, 15 bp, Brassica napus
  source 1..15: mol_type = genomic DNA
ggtctttcaactgag

SEQ ID NO: 526 - DNA, 16 bp, Brassica napus
  source 1..16: mol_type = genomic DNA
ggtctttcaactgagc

SEQ ID NO: 527 - DNA, 17 bp, Brassica napus
  source 1..17: mol_type = genomic DNA
ggtctttcaactgagct

SEQ ID NO: 528 - DNA, 18 bp, Brassica napus
  source 1..18: mol_type = genomic DNA
ggtctttcaactgagctc

SEQ ID NO: 529 - DNA, 19 bp, Brassica napus
  source 1..19: mol_type = genomic DNA
ggtctttcaactgagctca

SEQ ID NO: 530 - DNA, 20 bp, Brassica napus
  source 1..20: mol_type = genomic DNA
ggtctttcaactgagctcaa

SEQ ID NO: 531 - DNA, 21 bp, Brassica napus
  source 1..21: mol_type = genomic DNA
ggtctttcaactgagctcaat

SEQ ID NO: 532 - DNA, 22 bp, Brassica napus
  source 1..22: mol_type = genomic DNA
ggtctttcaactgagctcaata

SEQ ID NO: 533 - DNA, 23 bp, Brassica napus
  source 1..23: mol_type = genomic DNA
ggtctttcaactgagctcaatat

SEQ ID NO: 534 - DNA, 15 bp, Brassica napus
  source 1..15: mol_type = genomic DNA
gtctttcaactgagc

SEQ ID NO: 535 - DNA, 16 bp, Brassica napus
  source 1..16: mol_type = genomic DNA
gtctttcaactgagct

SEQ ID NO: 536 - DNA, 17 bp, Brassica napus
  source 1..17: mol_type = genomic DNA
gtctttcaactgagctc

SEQ ID NO: 537 - DNA, 18 bp, Brassica napus
  source 1..18: mol_type = genomic DNA
gtctttcaactgagctca

SEQ ID NO: 538 - DNA, 19 bp, Brassica napus
  source 1..19: mol_type = genomic DNA
gtctttcaactgagctcaa

SEQ ID NO: 539 - DNA, 20 bp, Brassica napus
  source 1..20: mol_type = genomic DNA
gtctttcaactgagctcaat

SEQ ID NO: 540 - DNA, 21 bp, Brassica napus
  source 1..21: mol_type = genomic DNA
gtctttcaactgagctcaata

SEQ ID NO: 541 - DNA, 22 bp, Brassica napus
  source 1..22: mol_type = genomic DNA
gtctttcaactgagctcaatat

SEQ ID NO: 542 - DNA, 23 bp, Brassica napus
  source 1..23: mol_type = genomic DNA
gtctttcaactgagctcaatata

SEQ ID NO: 543 - DNA, 15 bp, Brassica napus
  source 1..15: mol_type = genomic DNA
tctttcaactgagct

SEQ ID NO: 544 - DNA, 16 bp, Brassica napus
  source 1..16: mol_type = genomic DNA
tctttcaactgagctc

SEQ ID NO: 545 - DNA, 17 bp, Brassica napus
  source 1..17: mol_type = genomic DNA
tctttcaactgagctca

SEQ ID NO: 546 - DNA, 18 bp, Brassica napus
  source 1..18: mol_type = genomic DNA
tctttcaactgagctcaa

SEQ ID NO: 547 - DNA, 19 bp, Brassica napus
  source 1..19: mol_type = genomic DNA
tctttcaactgagctcaat

SEQ ID NO: 548 - DNA, 20 bp, Brassica napus
  source 1..20: mol_type = genomic DNA
tctttcaactgagctcaata

SEQ ID NO: 549 - DNA, 21 bp, Brassica napus
  source 1..21: mol_type = genomic DNA
tctttcaactgagctcaatat

SEQ ID NO: 550 - DNA, 22 bp, Brassica napus
  source 1..22: mol_type = genomic DNA
tctttcaactgagctcaatata

SEQ ID NO: 551 - DNA, 23 bp, Brassica napus
  source 1..23: mol_type = genomic DNA
tctttcaactgagctcaatatac

SEQ ID NO: 552 - DNA, 15 bp, Brassica napus
  source 1..15: mol_type = genomic DNA
ctttcaactgagctc

SEQ ID NO: 553 - DNA, 16 bp, Brassica napus
  source 1..16: mol_type = genomic DNA
ctttcaactgagctca

SEQ ID NO: 554 - DNA, 17 bp, Brassica napus
  source 1..17: mol_type = genomic DNA
ctttcaactgagctcaa

SEQ ID NO: 555 - DNA, 18 bp, Brassica napus
  source 1..18: mol_type = genomic DNA
ctttcaactgagctcaat

SEQ ID NO: 556 - DNA, 19 bp, Brassica napus
  source 1..19: mol_type = genomic DNA
ctttcaactgagctcaata

SEQ ID NO: 557 - DNA, 20 bp, Brassica napus
  source 1..20: mol_type = genomic DNA
ctttcaactgagctcaatat

SEQ ID NO: 558 - DNA, 21 bp, Brassica napus
  source 1..21: mol_type = genomic DNA
ctttcaactgagctcaatata

SEQ ID NO: 559 - DNA, 22 bp, Brassica napus
  source 1..22: mol_type = genomic DNA
ctttcaactgagctcaatatac

SEQ ID NO: 560 - DNA, 23 bp, Brassica napus
  source 1..23: mol_type = genomic DNA
ctttcaactgagctcaatataca

SEQ ID NO: 561 - DNA, 15 bp, Brassica napus
  source 1..15: mol_type = genomic DNA
tttcaactgagctca

SEQ ID NO: 562 - DNA, 16 bp, Brassica napus
  source 1..16: mol_type = genomic DNA
tttcaactgagctcaa

SEQ ID NO: 563 - DNA, 17 bp, Brassica napus
  source 1..17: mol_type = genomic DNA
tttcaactgagctcaat

SEQ ID NO: 564 - DNA, 18 bp, Brassica napus
  source 1..18: mol_type = genomic DNA
tttcaactgagctcaata

SEQ ID NO: 565 - DNA, 19 bp, Brassica napus
  source 1..19: mol_type = genomic DNA
tttcaactgagctcaatat

SEQ ID NO: 566 - DNA, 20 bp, Brassica napus
  source 1..20: mol_type = genomic DNA
tttcaactgagctcaatata

SEQ ID NO: 567 - DNA, 21 bp, Brassica napus
  source 1..21: mol_type = genomic DNA
tttcaactgagctcaatatac

SEQ ID NO: 568 - DNA, 22 bp, Brassica napus
  source 1..22: mol_type = genomic DNA
tttcaactgagctcaatataca

SEQ ID NO: 569 - DNA, 23 bp, Brassica napus
  source 1..23: mol_type = genomic DNA
tttcaactgagctcaatatacac

SEQ ID NO: 570 - DNA, 15 bp, Brassica napus
  source 1..15: mol_type = genomic DNA
ttcaactgagctcaa

SEQ ID NO: 571 - DNA, 16 bp, Brassica napus
  source 1..16: mol_type = genomic DNA
ttcaactgagctcaat

SEQ ID NO: 572 - DNA, 17 bp, Brassica napus
  source 1..17: mol_type = genomic DNA
ttcaactgagctcaata

SEQ ID NO: 573 - DNA, 18 bp, Brassica napus
  source 1..18: mol_type = genomic DNA
ttcaactgagctcaatat

SEQ ID NO: 574 - DNA, 19 bp, Brassica napus
  source 1..19: mol_type = genomic DNA
ttcaactgagctcaatata

SEQ ID NO: 575 - DNA, 20 bp, Brassica napus
  source 1..20: mol_type = genomic DNA
ttcaactgagctcaatatac

SEQ ID NO: 576 - DNA, 21 bp, Brassica napus
  source 1..21: mol_type = genomic DNA
ttcaactgagctcaatataca

SEQ ID NO: 577 - DNA, 22 bp, Brassica napus
  source 1..22: mol_type = genomic DNA
ttcaactgagctcaatatacac

SEQ ID NO: 578 - DNA, 23 bp, Brassica napus
  source 1..23: mol_type = genomic DNA
ttcaactgagctcaatatacaca

SEQ ID NO: 579 - DNA, 15 bp, Brassica napus
  source 1..15: mol_type = genomic DNA
tcaactgagctcaat

SEQ ID NO: 580 - DNA, 16 bp, Brassica napus
  source 1..16: mol_type = genomic DNA
tcaactgagctcaata

SEQ ID NO: 581 - DNA, 17 bp, Brassica napus
  source 1..17: mol_type = genomic DNA
tcaactgagctcaatat

SEQ ID NO: 582 - DNA, 18 bp, Brassica napus
  source 1..18: mol_type = genomic DNA
tcaactgagctcaatata

SEQ ID NO: 583 - DNA, 19 bp, Brassica napus
  source 1..19: mol_type = genomic DNA
tcaactgagctcaatatac

SEQ ID NO: 584 - DNA, 20 bp, Brassica napus
  source 1..20: mol_type = genomic DNA
tcaactgagctcaatataca

SEQ ID NO: 585 - DNA, 21 bp, Brassica napus
  source 1..21: mol_type = genomic DNA
tcaactgagctcaatatacac

SEQ ID NO: 586 - DNA, 22 bp, Brassica napus
  source 1..22: mol_type = genomic DNA
tcaactgagctcaatatacaca

SEQ ID NO: 587 - DNA, 23 bp, Brassica napus
  source 1..23: mol_type = genomic DNA
tcaactgagctcaatatacacaa

SEQ ID NO: 588 - DNA, 15 bp, Brassica napus
  source 1..15: mol_type = genomic DNA
caactgagctcaata

SEQ ID NO: 589 - DNA, 16 bp, Brassica napus
  source 1..16: mol_type = genomic DNA
caactgagctcaatat

SEQ ID NO: 590 - DNA, 17 bp, Brassica napus
  source 1..17: mol_type = genomic DNA
caactgagctcaatata

SEQ ID NO: 591 - DNA, 18 bp, Brassica napus
  source 1..18: mol_type = genomic DNA
caactgagctcaatatac

SEQ ID NO: 592 - DNA, 19 bp, Brassica napus
  source 1..19: mol_type = genomic DNA
caactgagctcaatataca

SEQ ID NO: 593 - DNA, 20 bp, Brassica napus
  source 1..20: mol_type = genomic DNA
caactgagctcaatatacac

SEQ ID NO: 594 - DNA, 21 bp, Brassica napus
  source 1..21: mol_type = genomic DNA
caactgagctcaatatacaca

SEQ ID NO: 595 - DNA, 22 bp, Brassica napus
  source 1..22: mol_type = genomic DNA
caactgagctcaatatacacaa

SEQ ID NO: 596 - DNA, 23 bp, Brassica napus
  source 1..23: mol_type = genomic DNA
caactgagctcaatatacacaag

SEQ ID NO: 597 - DNA, 15 bp, Brassica napus
  source 1..15: mol_type = genomic DNA
agtggccttcacgct

SEQ ID NO: 598 - DNA, 16 bp, Brassica napus
  source 1..16: mol_type = genomic DNA
agtggccttcacgctc

SEQ ID NO: 599 - DNA, 17 bp, Brassica napus
  source 1..17: mol_type = genomic DNA
agtggccttcacgctcc

SEQ ID NO: 600 - DNA, 18 bp, Brassica napus
  source 1..18: mol_type = genomic DNA
agtggccttcacgctccg

SEQ ID NO: 601 - DNA, 19 bp, Brassica napus
  source 1..19: mol_type = genomic DNA
agtggccttcacgctccga

SEQ ID NO: 602 - DNA, 20 bp, Brassica napus
  source 1..20: mol_type = genomic DNA
agtggccttcacgctccgat

SEQ ID NO: 603 - DNA, 21 bp, Brassica napus
  source 1..21: mol_type = genomic DNA
agtggccttcacgctccgatg

SEQ ID NO: 604 - DNA, 22 bp, Brassica napus
  source 1..22: mol_type = genomic DNA
agtggccttcacgctccgatgt

SEQ ID NO: 605 - DNA, 23 bp, Brassica napus
  source 1..23: mol_type = genomic DNA
agtggccttcacgctccgatgtc

SEQ ID NO: 606 - DNA, 15 bp, Brassica napus
  source 1..15: mol_type = genomic DNA
aactgagctcaatat

SEQ ID NO: 607 - DNA, 16 bp, Brassica napus
  source 1..16: mol_type = genomic DNA
aactgagctcaatata

SEQ ID NO: 608 - DNA, 17 bp, Brassica napus
  source 1..17: mol_type = genomic DNA
aactgagctcaatatac

SEQ ID NO: 609 - DNA, 18 bp, Brassica napus
  source 1..18: mol_type = genomic DNA
aactgagctcaatataca

SEQ ID NO: 610 - DNA, 19 bp, Brassica napus
  source 1..19: mol_type = genomic DNA
aactgagctcaatatacac

SEQ ID NO: 611 - DNA, 20 bp, Brassica napus
  source 1..20: mol_type = genomic DNA
aactgagctcaatatacaca

SEQ ID NO: 612 - DNA, 21 bp, Brassica napus
  source 1..21: mol_type = genomic DNA
aactgagctcaatatacacaa

SEQ ID NO: 613 - DNA, 22 bp, Brassica napus
  source 1..22: mol_type = genomic DNA
aactgagctcaatatacacaag

SEQ ID NO: 614 - DNA, 23 bp, Brassica napus
  source 1..23: mol_type = genomic DNA
aactgagctcaatatacacaaga

SEQ ID NO: 615 - DNA, 15 bp, Brassica napus
  source 1..15: mol_type = genomic DNA
actgagctcaatata

SEQ ID NO: 616 - DNA, 16 bp, Brassica napus
  source 1..16: mol_type = genomic DNA
actgagctcaatatac

SEQ ID NO: 617 - DNA, 17 bp, Brassica napus
  source 1..17: mol_type = genomic DNA
actgagctcaatataca

SEQ ID NO: 618 - DNA, 18 bp, Brassica napus
  source 1..18: mol_type = genomic DNA
actgagctcaatatacac

SEQ ID NO: 619 - DNA, 19 bp, Brassica napus
  source 1..19: mol_type = genomic DNA
actgagctcaatatacaca

SEQ ID NO: 620 - DNA, 20 bp, Brassica napus
  source 1..20: mol_type = genomic DNA
actgagctcaatatacacaa

SEQ ID NO: 621 - DNA, 21 bp, Brassica napus
  source 1..21: mol_type = genomic DNA
actgagctcaatatacacaag

SEQ ID NO: 622 - DNA, 22 bp, Brassica napus
  source 1..22: mol_type = genomic DNA
actgagctcaatatacacaaga

SEQ ID NO: 623 - DNA, 23 bp, Brassica napus
  source 1..23: mol_type = genomic DNA
actgagctcaatatacacaagaa

SEQ ID NO: 624 - DNA, 15 bp, Brassica napus
  source 1..15: mol_type = genomic DNA
ctgagctcaatatac

SEQ ID NO: 625 - DNA, 16 bp, Brassica napus
  source 1..16: mol_type = genomic DNA
ctgagctcaatataca

SEQ ID NO: 626 - DNA, 17 bp, Brassica napus
  source 1..17: mol_type = genomic DNA
ctgagctcaatatacac

SEQ ID NO: 627 - DNA, 18 bp, Brassica napus
  source 1..18: mol_type = genomic DNA
ctgagctcaatatacaca

SEQ ID NO: 628 - DNA, 19 bp, Brassica napus
  source 1..19: mol_type = genomic DNA
ctgagctcaatatacacaa

SEQ ID NO: 629 - DNA, 20 bp, Brassica napus
  source 1..20: mol_type = genomic DNA
ctgagctcaatatacacaag

SEQ ID NO: 630 - DNA, 21 bp, Brassica napus
  source 1..21: mol_type = genomic DNA
ctgagctcaatatacacaaga

SEQ ID NO: 631 - DNA, 22 bp, Brassica napus
  source 1..22: mol_type = genomic DNA
ctgagctcaatatacacaagaa

SEQ ID NO: 632 - DNA, 23 bp, Brassica napus
  source 1..23: mol_type = genomic DNA
ctgagctcaatatacacaagaaa

SEQ ID NO: 633 - DNA, 15 bp, Brassica napus
  source 1..15: mol_type = genomic DNA
tgagctcaatataca

SEQ ID NO: 634 - DNA, 16 bp, Brassica napus
  source 1..16: mol_type = genomic DNA
tgagctcaatatacac

SEQ ID NO: 635 - DNA, 17 bp, Brassica napus
  source 1..17: mol_type = genomic DNA
tgagctcaatatacaca

SEQ ID NO: 636 - DNA, 18 bp, Brassica napus
  source 1..18: mol_type = genomic DNA
tgagctcaatatacacaa

SEQ ID NO: 637 - DNA, 19 bp, Brassica napus
  source 1..19: mol_type = genomic DNA
tgagctcaatatacacaag

SEQ ID NO: 638 - DNA, 20 bp, Brassica napus
  source 1..20: mol_type = genomic DNA
tgagctcaatatacacaaga

SEQ ID NO: 639 - DNA, 21 bp, Brassica napus
  source 1..21: mol_type = genomic DNA
tgagctcaatatacacaagaa

SEQ ID NO: 640 - DNA, 22 bp, Brassica napus
  source 1..22: mol_type = genomic DNA
tgagctcaatatacacaagaaa

SEQ ID NO: 641 - DNA, 23 bp, Brassica napus
  source 1..23: mol_type = genomic DNA
tgagctcaatatacacaagaaac

SEQ ID NO: 642 - DNA, 15 bp, Brassica napus
  source 1..15: mol_type = genomic DNA
gagctcaatatacac

SEQ ID NO: 643 - DNA, 16 bp, Brassica napus
  source 1..16: mol_type = genomic DNA
gagctcaatatacaca

SEQ ID NO: 644 - DNA, 17 bp, Brassica napus
  source 1..17: mol_type = genomic DNA
gagctcaatatacacaa

SEQ ID NO: 645 - DNA, 18 bp, Brassica napus
  source 1..18: mol_type = genomic DNA
gagctcaatatacacaag

SEQ ID NO: 646 - DNA, 19 bp, Brassica napus
  source 1..19: mol_type = genomic DNA
gagctcaatatacacaaga

SEQ ID NO: 647 - DNA, 20 bp, Brassica napus
  source 1..20: mol_type = genomic DNA
gagctcaatatacacaagaa

SEQ ID NO: 648 - DNA, 21 bp, Brassica napus
  source 1..21: mol_type = genomic DNA
gagctcaatatacacaagaaa

SEQ ID NO: 649 - DNA, 22 bp, Brassica napus
  source 1..22: mol_type = genomic DNA
gagctcaatatacacaagaaac

SEQ ID NO: 650 - DNA, 23 bp, Brassica napus
  source 1..23: mol_type = genomic DNA
gagctcaatatacacaagaaaca

SEQ ID NO: 651 - DNA, 15 bp, Brassica napus
  source 1..15: mol_type = genomic DNA
agctcaatatacaca

SEQ ID NO: 652 - DNA, 16 bp, Brassica napus
  source 1..16: mol_type = genomic DNA
agctcaatatacacaa

SEQ ID NO: 653 - DNA, 17 bp, Brassica napus
  source 1..17: mol_type = genomic DNA
agctcaatatacacaag

SEQ ID NO: 654 - DNA, 18 bp, Brassica napus
  source 1..18: mol_type = genomic DNA
agctcaatatacacaaga

SEQ ID NO: 655 - DNA, 19 bp, Brassica napus
  source 1..19: mol_type = genomic DNA
agctcaatatacacaagaa

SEQ ID NO: 656 - DNA, 20 bp, Brassica napus
  source 1..20: mol_type = genomic DNA
agctcaatatacacaagaaa

SEQ ID NO: 657 - DNA, 21 bp, Brassica napus
  source 1..21: mol_type = genomic DNA
agctcaatatacacaagaaac

SEQ ID NO: 658 - DNA, 22 bp, Brassica napus
  source 1..22: mol_type = genomic DNA
agctcaatatacacaagaaaca

SEQ ID NO: 659 - DNA, 23 bp, Brassica napus
  source 1..23: mol_type = genomic DNA
agctcaatatacacaagaaacac

SEQ ID NO: 660 - DNA, 15 bp, Brassica napus
  source 1..15: mol_type = genomic DNA
gctcaatatacacaa

SEQ ID NO: 661 - DNA, 16 bp, Brassica napus
  source 1..16: mol_type = genomic DNA
gctcaatatacacaag

SEQ ID NO: 662 - DNA, 17 bp, Brassica napus
  source 1..17: mol_type = genomic DNA
gctcaatatacacaaga

SEQ ID NO: 663 - DNA, 18 bp, Brassica napus
  source 1..18: mol_type = genomic DNA
gctcaatatacacaagaa

SEQ ID NO: 664 - DNA, 19 bp, Brassica napus
  source 1..19: mol_type = genomic DNA
gctcaatatacacaagaaa

SEQ ID NO: 665 - DNA, 20 bp, Brassica napus
  source 1..20: mol_type = genomic DNA
gctcaatatacacaagaaac

SEQ ID NO: 666 - DNA, 21 bp, Brassica napus
  source 1..21: mol_type = genomic DNA
gctcaatatacacaagaaaca

SEQ ID NO: 667 - DNA, 22 bp, Brassica napus
  source 1..22: mol_type = genomic DNA
gctcaatatacacaagaaacac

SEQ ID NO: 668 - DNA, 23 bp, Brassica napus
  source 1..23: mol_type = genomic DNA
gctcaatatacacaagaaacacc

SEQ ID NO: 669 - DNA, 15 bp, Brassica napus
  source 1..15: mol_type = genomic DNA
ctcaatatacacaag

SEQ ID NO: 670 - DNA, 16 bp, Brassica napus
  source 1..16: mol_type = genomic DNA
ctcaatatacacaaga

SEQ ID NO: 671 - DNA, 17 bp, Brassica napus
  source 1..17: mol_type = genomic DNA
ctcaatatacacaagaa

SEQ ID NO: 672 - DNA, 18 bp, Brassica napus
  source 1..18: mol_type = genomic DNA
ctcaatatacacaagaaa

SEQ ID NO: 673 - DNA, 19 bp, Brassica napus
  source 1..19: mol_type = genomic DNA
ctcaatatacacaagaaac

SEQ ID NO: 674 - DNA, 20 bp, Brassica napus
  source 1..20: mol_type = genomic DNA
ctcaatatacacaagaaaca

SEQ ID NO: 675 - DNA, 21 bp, Brassica napus
  source 1..21: mol_type = genomic DNA
ctcaatatacacaagaaacac

SEQ ID NO: 676 - DNA, 22 bp, Brassica napus
  source 1..22: mol_type = genomic DNA
ctcaatatacacaagaaacacc

SEQ ID NO: 677 - DNA, 23 bp, Brassica napus
  source 1..23: mol_type = genomic DNA
ctcaatatacacaagaaacacct

SEQ ID NO: 678 - DNA, 15 bp, Brassica napus
  source 1..15: mol_type = genomic DNA
tcaatatacacaaga

SEQ ID NO: 679 - DNA, 16 bp, Brassica napus
  source 1..16: mol_type = genomic DNA
tcaatatacacaagaa

SEQ ID NO: 680 - DNA, 17 bp, Brassica napus
  source 1..17: mol_type = genomic DNA
tcaatatacacaagaaa

SEQ ID NO: 681 - DNA, 18 bp, Brassica napus
  source 1..18: mol_type = genomic DNA
tcaatatacacaagaaac

SEQ ID NO: 682 - DNA, 19 bp, Brassica napus
  source 1..19: mol_type = genomic DNA
tcaatatacacaagaaaca

SEQ ID NO: 683 - DNA, 20 bp, Brassica napus
  source 1..20: mol_type = genomic DNA
tcaatatacacaagaaacac

SEQ ID NO: 684 - DNA, 21 bp, Brassica napus
  source 1..21: mol_type = genomic DNA
tcaatatacacaagaaacacc

SEQ ID NO: 685 - DNA, 22 bp, Brassica napus
  source 1..22: mol_type = genomic DNA
tcaatatacacaagaaacacct

SEQ ID NO: 686 - DNA, 23 bp, Brassica napus
  source 1..23: mol_type = genomic DNA
tcaatatacacaagaaacacctt

SEQ ID NO: 687 - DNA, 15 bp, Brassica napus
  source 1..15: mol_type = genomic DNA
caatatacacaagaa

SEQ ID NO: 688 - DNA, 16 bp, Brassica napus
  source 1..16: mol_type = genomic DNA
caatatacacaagaaa

SEQ ID NO: 689 - DNA, 17 bp, Brassica napus
  source 1..17: mol_type = genomic DNA
caatatacacaagaaac

SEQ ID NO: 690 - DNA, 18 bp, Brassica napus
  source 1..18: mol_type = genomic DNA
caatatacacaagaaaca

SEQ ID NO: 691 - DNA, 19 bp, Brassica napus
  source 1..19: mol_type = genomic DNA
caatatacacaagaaacac

SEQ ID NO: 692 - DNA, 20 bp, Brassica napus
  source 1..20: mol_type = genomic DNA
caatatacacaagaaacacc

SEQ ID NO: 693 - DNA, 21 bp, Brassica napus
  source 1..21: mol_type = genomic DNA
caatatacacaagaaacacct

SEQ ID NO: 694 - DNA, 22 bp, Brassica napus
  source 1..22: mol_type = genomic DNA
caatatacacaagaaacacctt

SEQ ID NO: 695 - DNA, 23 bp, Brassica napus
  source 1..23: mol_type = genomic DNA
caatatacacaagaaacacctta

SEQ ID NO: 696 - DNA, 15 bp, Brassica napus
  source 1..15: mol_type = genomic DNA
gtggccttcacgctc

SEQ ID NO: 697 - DNA, 16 bp, Brassica napus
  source 1..16: mol_type = genomic DNA
gtggccttcacgctcc

SEQ ID NO: 698 - DNA, 17 bp, Brassica napus
  source 1..17: mol_type = genomic DNA
gtggccttcacgctccg

SEQ ID NO: 699 - DNA, 18 bp, Brassica napus
  source 1..18: mol_type = genomic DNA
gtggccttcacgctccga

SEQ ID NO: 700 - DNA, 19 bp, Brassica napus
  source 1..19: mol_type = genomic DNA
gtggccttcacgctccgat

SEQ ID NO: 701 - DNA, 20 bp, Brassica napus
  source 1..20: mol_type = genomic DNA
gtggccttcacgctccgatg

SEQ ID NO: 702 - DNA, 21 bp, Brassica napus
  source 1..21: mol_type = genomic DNA
gtggccttcacgctccgatgt

SEQ ID NO: 703 - DNA, 22 bp, Brassica napus
  source 1..22: mol_type = genomic DNA
gtggccttcacgctccgatgtc

SEQ ID NO: 704 - DNA, 23 bp, Brassica napus
  source 1..23: mol_type = genomic DNA
gtggccttcacgctccgatgtct

SEQ ID NO: 705 - DNA, 15 bp, Brassica napus
  source 1..15: mol_type = genomic DNA
aatatacacaagaaa

SEQ ID NO: 706 - DNA, 16 bp, Brassica napus
  source 1..16: mol_type = genomic DNA
aatatacacaagaaac

SEQ ID NO: 707 - DNA, 17 bp, Brassica napus
  source 1..17: mol_type = genomic DNA
aatatacacaagaaaca

SEQ ID NO: 708 - DNA, 18 bp, Brassica napus
  source 1..18: mol_type = genomic DNA
aatatacacaagaaacac

SEQ ID NO: 709 - DNA, 19 bp, Brassica napus
  source 1..19: mol_type = genomic DNA
aatatacacaagaaacacc

SEQ ID NO: 710 - DNA, 20 bp, Brassica napus
  source 1..20: mol_type = genomic DNA
aatatacacaagaaacacct

SEQ ID NO: 711 - DNA, 21 bp, Brassica napus
  source 1..21: mol_type = genomic DNA
aatatacacaagaaacacctt

SEQ ID NO: 712 - DNA, 22 bp, Brassica napus
  source 1..22: mol_type = genomic DNA
aatatacacaagaaacacctta

SEQ ID NO: 713 - DNA, 23 bp, Brassica napus
  source 1..23: mol_type = genomic DNA
aatatacacaagaaacaccttac

SEQ ID NO: 714 - DNA, 15 bp, Brassica napus
  source 1..15: mol_type = genomic DNA
atatacacaagaaac

SEQ ID NO: 715 - DNA, 16 bp, Brassica napus
  source 1..16: mol_type = genomic DNA
atatacacaagaaaca

SEQ ID NO: 716 - DNA, 17 bp, Brassica napus
  source 1..17: mol_type = genomic DNA
atatacacaagaaacac

SEQ ID NO: 717 - DNA, 18 bp, Brassica napus
  source 1..18: mol_type = genomic DNA
atatacacaagaaacacc

SEQ ID NO: 718 - DNA, 19 bp, Brassica napus
  source 1..19: mol_type = genomic DNA
atatacacaagaaacacct

SEQ ID NO: 719 - DNA, 20 bp, Brassica napus
  source 1..20: mol_type = genomic DNA
atatacacaagaaacacctt

SEQ ID NO: 720 - DNA, 21 bp, Brassica napus
  source 1..21: mol_type = genomic DNA
atatacacaagaaacacctta

SEQ ID NO: 721 - DNA, 22 bp, Brassica napus
  source 1..22: mol_type = genomic DNA
atatacacaagaaacaccttac

SEQ ID NO: 722 - DNA, 23 bp, Brassica napus
  source 1..23: mol_type = genomic DNA
atatacacaagaaacaccttact

SEQ ID NO: 723 - DNA, 15 bp, Brassica napus
  source 1..15: mol_type = genomic DNA
tatacacaagaaaca

SEQ ID NO: 724 - DNA, 16 bp, Brassica napus
  source 1..16: mol_type = genomic DNA
tatacacaagaaacac

SEQ ID NO: 725 - DNA, 17 bp, Brassica napus
  source 1..17: mol_type = genomic DNA
tatacacaagaaacacc

SEQ ID NO: 726 - DNA, 18 bp, Brassica napus
  source 1..18: mol_type = genomic DNA
tatacacaagaaacacct

SEQ ID NO: 727 - DNA, 19 bp, Brassica napus
  source 1..19: mol_type = genomic DNA
tatacacaagaaacacctt

SEQ ID NO: 728 - DNA, 20 bp, Brassica napus
  source 1..20: mol_type = genomic DNA
tatacacaagaaacacctta

SEQ ID NO: 729 - DNA, 21 bp, Brassica napus
  source 1..21: mol_type = genomic DNA
tatacacaagaaacaccttac

SEQ ID NO: 730 - DNA, 22 bp, Brassica napus
  source 1..22: mol_type = genomic DNA
tatacacaagaaacaccttact

SEQ ID NO: 731 - DNA, 23 bp, Brassica napus
  source 1..23: mol_type = genomic DNA
tatacacaagaaacaccttactt

SEQ ID NO: 732 - DNA, 15 bp, Brassica napus
  source 1..15: mol_type = genomic DNA
atacacaagaaacac

SEQ ID NO: 733 - DNA, 16 bp, Brassica napus
  source 1..16: mol_type = genomic DNA
atacacaagaaacacc

SEQ ID NO: 734 - DNA, 17 bp, Brassica napus
  source 1..17: mol_type = genomic DNA
atacacaagaaacacct

SEQ ID NO: 735 - DNA, 18 bp, Brassica napus
  source 1..18: mol_type = genomic DNA
atacacaagaaacacctt

SEQ ID NO: 736 - DNA, 19 bp, Brassica napus
  source 1..19: mol_type = genomic DNA
atacacaagaaacacctta

SEQ ID NO: 737 - DNA, 20 bp, Brassica napus
  source 1..20: mol_type = genomic DNA
atacacaagaaacaccttac

SEQ ID NO: 738 - DNA, 21 bp, Brassica napus
  source 1..21: mol_type = genomic DNA
atacacaagaaacaccttact

SEQ ID NO: 739 - DNA, 22 bp, Brassica napus
  source 1..22: mol_type = genomic DNA
atacacaagaaacaccttactt

SEQ ID NO: 740 - DNA, 23 bp, Brassica napus
  source 1..23: mol_type = genomic DNA
atacacaagaaacaccttacttt

SEQ ID NO: 741 - DNA, 15 bp, Brassica napus
  source 1..15: mol_type = genomic DNA
tacacaagaaacacc

SEQ ID NO: 742 - DNA, 16 bp, Brassica napus
  source 1..16: mol_type = genomic DNA
tacacaagaaacacct

SEQ ID NO: 743 - DNA, 17 bp, Brassica napus
  source 1..17: mol_type = genomic DNA
tacacaagaaacacctt

SEQ ID NO: 744 - DNA, 18 bp, Brassica napus
  source 1..18: mol_type = genomic DNA
tacacaagaaacacctta

SEQ ID NO: 745 - DNA, 19 bp, Brassica napus
  source 1..19: mol_type = genomic DNA
tacacaagaaacaccttac

SEQ ID NO: 746 - DNA, 20 bp, Brassica napus
  source 1..20: mol_type = genomic DNA
tacacaagaaacaccttact

SEQ ID NO: 747 - DNA, 21 bp, Brassica napus
  source 1..21: mol_type = genomic DNA
tacacaagaaacaccttactt

SEQ ID NO: 748 - DNA, 22 bp, Brassica napus
  source 1..22: mol_type = genomic DNA
tacacaagaaacaccttacttt

SEQ ID NO: 749 - DNA, 23 bp, Brassica napus
  source 1..23: mol_type = genomic DNA
tacacaagaaacaccttactttg

SEQ ID NO: 750 - DNA, 15 bp, Brassica napus
  source 1..15: mol_type = genomic DNA
acacaagaaacacct

SEQ ID NO: 751 - DNA, 16 bp, Brassica napus
  source 1..16: mol_type = genomic DNA
acacaagaaacacctt

SEQ ID NO: 752 - DNA, 17 bp, Brassica napus
  source 1..17: mol_type = genomic DNA
acacaagaaacacctta

SEQ ID NO: 753 - DNA, 18 bp, Brassica napus
  source 1..18: mol_type = genomic DNA
acacaagaaacaccttac

SEQ ID NO: 754 - DNA, 19 bp, Brassica napus
  source 1..19: mol_type = genomic DNA
acacaagaaacaccttact

SEQ ID NO: 755 - DNA, 20 bp, Brassica napus
  source 1..20: mol_type = genomic DNA
acacaagaaacaccttactt

SEQ ID NO: 756 - DNA, 21 bp, Brassica napus
  source 1..21: mol_type = genomic DNA
acacaagaaacaccttacttt

SEQ ID NO: 757 - DNA, 22 bp, Brassica napus
  source 1..22: mol_type = genomic DNA
acacaagaaacaccttactttg

SEQ ID NO: 758 - DNA, 23 bp, Brassica napus
  source 1..23: mol_type = genomic DNA
acacaagaaacaccttactttgt

SEQ ID NO: 759 - DNA, 15 bp, Brassica napus
  source 1..15: mol_type = genomic DNA
cacaagaaacacctt

SEQ ID NO: 760 - DNA, 16 bp, Brassica napus
  source 1..16: mol_type = genomic DNA
cacaagaaacacctta

SEQ ID NO: 761 - DNA, 17 bp, Brassica napus
  source 1..17: mol_type = genomic DNA
cacaagaaacaccttac

SEQ ID NO: 762 - DNA, 18 bp, Brassica napus
  source 1..18: mol_type = genomic DNA
cacaagaaacaccttact

SEQ ID NO: 763 - DNA, 19 bp, Brassica napus
  source 1..19: mol_type = genomic DNA
cacaagaaacaccttactt

SEQ ID NO: 764 - DNA, 20 bp, Brassica napus
  source 1..20: mol_type = genomic DNA
cacaagaaacaccttacttt

SEQ ID NO: 765 - DNA, 21 bp, Brassica napus
  source 1..21: mol_type = genomic DNA
cacaagaaacaccttactttg

SEQ ID NO: 766 - DNA, 22 bp, Brassica napus
  source 1..22: mol_type = genomic DNA
cacaagaaacaccttactttgt

SEQ ID NO: 767 - DNA, 23 bp, Brassica napus
  source 1..23: mol_type = genomic DNA
cacaagaaacaccttactttgtt

SEQ ID NO: 768 - DNA, 15 bp, Brassica napus
  source 1..15: mol_type = genomic DNA
acaagaaacacctta

SEQ ID NO: 769 - DNA, 16 bp, Brassica napus
  source 1..16: mol_type = genomic DNA
acaagaaacaccttac

SEQ ID NO: 770 - DNA, 17 bp, Brassica napus
  source 1..17: mol_type = genomic DNA
acaagaaacaccttact

SEQ ID NO: 771 - DNA, 18 bp, Brassica napus
  source 1..18: mol_type = genomic DNA
acaagaaacaccttactt

SEQ ID NO: 772 - DNA, 19 bp, Brassica napus
  source 1..19: mol_type = genomic DNA
acaagaaacaccttacttt

SEQ ID NO: 773 - DNA, 20 bp, Brassica napus
  source 1..20: mol_type = genomic DNA
acaagaaacaccttactttg

SEQ ID NO: 774 - DNA, 21 bp, Brassica napus
  source 1..21: mol_type = genomic DNA
acaagaaacaccttactttgt

SEQ ID NO: 775 - DNA, 22 bp, Brassica napus
  source 1..22: mol_type = genomic DNA
acaagaaacaccttactttgtt

SEQ ID NO: 776 - DNA, 23 bp, Brassica napus
  source 1..23: mol_type = genomic DNA
acaagaaacaccttactttgtta

SEQ ID NO: 777 - DNA, 15 bp, Brassica napus
  source 1..15: mol_type = genomic DNA
caagaaacaccttac

SEQ ID NO: 778 - DNA, 16 bp, Brassica napus
  source 1..16: mol_type = genomic DNA
caagaaacaccttact

SEQ ID NO: 779 - DNA, 17 bp, Brassica napus
  source 1..17: mol_type = genomic DNA
caagaaacaccttactt

SEQ ID NO: 780 - DNA, 18 bp, Brassica napus
  source 1..18: mol_type = genomic DNA
caagaaacaccttacttt

SEQ ID NO: 781 - DNA, 19 bp, Brassica napus
  source 1..19: mol_type = genomic DNA
caagaaacaccttactttg

SEQ ID NO: 782 - DNA, 20 bp, Brassica napus
  source 1..20: mol_type = genomic DNA
caagaaacaccttactttgt

SEQ ID NO: 783 - DNA, 21 bp, Brassica napus
  source 1..21: mol_type = genomic DNA
caagaaacaccttactttgtt

SEQ ID NO: 784 - DNA, 22 bp, Brassica napus
  source 1..22: mol_type = genomic DNA
caagaaacaccttactttgtta

SEQ ID NO: 785 - DNA, 23 bp, Brassica napus
  source 1..23: mol_type = genomic DNA
caagaaacaccttactttgttaa

SEQ ID NO: 786 - DNA, 15 bp, Brassica napus
  source 1..15: mol_type = genomic DNA
aagaaacaccttact

SEQ ID NO: 787 - DNA, 16 bp, Brassica napus
  source 1..16: mol_type = genomic DNA
aagaaacaccttactt

SEQ ID NO: 788 - DNA, 17 bp, Brassica napus
  source 1..17: mol_type = genomic DNA
aagaaacaccttacttt

SEQ ID NO: 789 - DNA, 18 bp, Brassica napus
  source 1..18: mol_type = genomic DNA
aagaaacaccttactttg

SEQ ID NO: 790 - DNA, 19 bp, Brassica napus
  source 1..19: mol_type = genomic DNA
aagaaacaccttactttgt

SEQ ID NO: 791 - DNA, 20 bp, Brassica napus
  source 1..20: mol_type = genomic DNA
aagaaacaccttactttgtt

SEQ ID NO: 792 - DNA, 21 bp, Brassica napus
  source 1..21: mol_type = genomic DNA
aagaaacaccttactttgtta

SEQ ID NO: 793 - DNA, 22 bp, Brassica napus
  source 1..22: mol_type = genomic DNA
aagaaacaccttactttgttaa

SEQ ID NO: 794 - DNA, 23 bp, Brassica napus
  source 1..23: mol_type = genomic DNA
aagaaacaccttactttgttaaa

SEQ ID NO: 795 - DNA, 15 bp, Brassica napus
  source 1..15: mol_type = genomic DNA
tggccttcacgctcc

SEQ ID NO: 796 - DNA, 16 bp, Brassica napus
  source 1..16: mol_type = genomic DNA
tggccttcacgctccg

SEQ ID NO: 797 - DNA, 17 bp, Brassica napus
  source 1..17: mol_type = genomic DNA
tggccttcacgctccga

SEQ ID NO: 798 - DNA, 18 bp, Brassica napus
  source 1..18: mol_type = genomic DNA
tggccttcacgctccgat

SEQ ID NO: 799 - DNA, 19 bp, Brassica napus
  source 1..19: mol_type = genomic DNA
tggccttcacgctccgatg

SEQ ID NO: 800 - DNA, 20 bp, Brassica napus
  source 1..20: mol_type = genomic DNA
tggccttcacgctccgatgt

SEQ ID NO: 801 - DNA, 21 bp, Brassica napus
  source 1..21: mol_type = genomic DNA
tggccttcacgctccgatgtc

SEQ ID NO: 802 - DNA, 22 bp, Brassica napus
  source 1..22: mol_type = genomic DNA
tggccttcacgctccgatgtct

SEQ ID NO: 803 - DNA, 23 bp, Brassica napus
  source 1..23: mol_type = genomic DNA
tggccttcacgctccgatgtctt

SEQ ID NO: 804 - DNA, 15 bp, Brassica napus
  source 1..15: mol_type = genomic DNA
agaaacaccttactt

SEQ ID NO: 805 - DNA, 16 bp, Brassica napus
  source 1..16: mol_type = genomic DNA
agaaacaccttacttt

SEQ ID NO: 806 - DNA, 17 bp, Brassica napus
  source 1..17: mol_type = genomic DNA
agaaacaccttactttg

SEQ ID NO: 807 - DNA, 18 bp, Brassica napus
  source 1..18: mol_type = genomic DNA
agaaacaccttactttgt

SEQ ID NO: 808 - DNA, 19 bp, Brassica napus
  source 1..19: mol_type = genomic DNA
agaaacaccttactttgtt

SEQ ID NO: 809 - DNA, 20 bp, Brassica napus
  source 1..20: mol_type = genomic DNA
agaaacaccttactttgtta

SEQ ID NO: 810 - DNA, 21 bp, Brassica napus
  source 1..21: mol_type = genomic DNA
agaaacaccttactttgttaa

SEQ ID NO: 811 - DNA, 22 bp, Brassica napus
  source 1..22: mol_type = genomic DNA
agaaacaccttactttgttaaa

SEQ ID NO: 812 - DNA, 23 bp, Brassica napus
  source 1..23: mol_type = genomic DNA
agaaacaccttactttgttaaat

SEQ ID NO: 813 - DNA, 15 bp, Brassica napus
  source 1..15: mol_type = genomic DNA
gaaacaccttacttt

SEQ ID NO: 814 - DNA, 16 bp, Brassica napus
  source 1..16: mol_type = genomic DNA
gaaacaccttactttg

SEQ ID NO: 815 - DNA, 17 bp, Brassica napus
  source 1..17: mol_type = genomic DNA
gaaacaccttactttgt

SEQ ID NO: 816 - DNA, 18 bp, Brassica napus
  source 1..18: mol_type = genomic DNA
gaaacaccttactttgtt

SEQ ID NO: 817 - DNA, 19 bp, Brassica napus
  source 1..19: mol_type = genomic DNA
gaaacaccttactttgtta

SEQ ID NO: 818 - DNA, 20 bp, Brassica napus
  source 1..20: mol_type = genomic DNA
gaaacaccttactttgttaa

SEQ ID NO: 819 - DNA, 21 bp, Brassica napus
  source 1..21: mol_type = genomic DNA
gaaacaccttactttgttaaa

SEQ ID NO: 820 - DNA, 22 bp, Brassica napus
  source 1..22: mol_type = genomic DNA
gaaacaccttactttgttaaat

SEQ ID NO: 821 - DNA, 23 bp, Brassica napus
  source 1..23: mol_type = genomic DNA
gaaacaccttactttgttaaatt

SEQ ID NO: 822 - DNA, 15 bp, Brassica napus
  source 1..15: mol_type = genomic DNA
aaacaccttactttg

SEQ ID NO: 823 - DNA, 16 bp, Brassica napus
  source 1..16: mol_type = genomic DNA
aaacaccttactttgt

SEQ ID NO: 824 - DNA, 17 bp, Brassica napus
  source 1..17: mol_type = genomic DNA
aaacaccttactttgtt

SEQ ID NO: 825 - DNA, 18 bp, Brassica napus
  source 1..18: mol_type = genomic DNA
aaacaccttactttgtta

SEQ ID NO: 826 - DNA, 19 bp, Brassica napus
  source 1..19: mol_type = genomic DNA
aaacaccttactttgttaa

SEQ ID NO: 827 - DNA, 20 bp, Brassica napus
  source 1..20: mol_type = genomic DNA
aaacaccttactttgttaaa

SEQ ID NO: 828 - DNA, 21 bp, Brassica napus
  source 1..21: mol_type = genomic DNA
aaacaccttactttgttaaat

SEQ ID NO: 829 - DNA, 22 bp, Brassica napus
  source 1..22: mol_type = genomic DNA
aaacaccttactttgttaaatt

SEQ ID NO: 830 - DNA, 23 bp, Brassica napus
  source 1..23: mol_type = genomic DNA
aaacaccttactttgttaaattt

SEQ ID NO: 831 - DNA, 15 bp, Brassica napus
  source 1..15: mol_type = genomic DNA
aacaccttactttgt

SEQ ID NO: 832 - DNA, 16 bp, Brassica napus
  source 1..16: mol_type = genomic DNA
aacaccttactttgtt

SEQ ID NO: 833 - DNA, 17 bp, Brassica napus
  source 1..17: mol_type = genomic DNA
aacaccttactttgtta

SEQ ID NO: 834 - DNA, 18 bp, Brassica napus
  source 1..18: mol_type = genomic DNA
aacaccttactttgttaa

SEQ ID NO: 835 - DNA, 19 bp, Brassica napus
  source 1..19: mol_type = genomic DNA
aacaccttactttgttaaa

SEQ ID NO: 836 - DNA, 20 bp, Brassica napus
  source 1..20: mol_type = genomic DNA
aacaccttactttgttaaat

SEQ ID NO: 837 - DNA, 21 bp, Brassica napus
  source 1..21: mol_type = genomic DNA
aacaccttactttgttaaatt

SEQ ID NO: 838 - DNA, 22 bp, Brassica napus
  source 1..22: mol_type = genomic DNA
aacaccttactttgttaaattt

SEQ ID NO: 839 - DNA, 23 bp, Brassica napus
  source 1..23: mol_type = genomic DNA
aacaccttactttgttaaattta

SEQ ID NO: 840 - DNA, 15 bp, Brassica napus
  source 1..15: mol_type = genomic DNA
acaccttactttgtt

SEQ ID NO: 841 - DNA, 16 bp, Brassica napus
  source 1..16: mol_type = genomic DNA
acaccttactttgtta

SEQ ID NO: 842 - DNA, 17 bp, Brassica napus
  source 1..17: mol_type = genomic DNA
acaccttactttgttaa

SEQ ID NO: 843 - DNA, 18 bp, Brassica napus
  source 1..18: mol_type = genomic DNA
acaccttactttgttaaa

SEQ ID NO: 844 - DNA, 19 bp, Brassica napus
  source 1..19: mol_type = genomic DNA
acaccttactttgttaaat

SEQ ID NO: 845 - DNA, 20 bp, Brassica napus
  source 1..20: mol_type = genomic DNA
acaccttactttgttaaatt

SEQ ID NO: 846 - DNA, 21 bp, Brassica napus
  source 1..21: mol_type = genomic DNA
acaccttactttgttaaattt

SEQ ID NO: 847 - DNA, 22 bp, Brassica napus
  source 1..22: mol_type = genomic DNA
acaccttactttgttaaattta

SEQ ID NO: 848 - DNA, 23 bp, Brassica napus
  source 1..23: mol_type = genomic DNA
acaccttactttgttaaatttaa

SEQ ID NO: 849 - DNA, 15 bp, Brassica napus
  source 1..15: mol_type = genomic DNA
caccttactttgtta

SEQ ID NO: 850 - DNA, 16 bp, Brassica napus
  source 1..16: mol_type = genomic DNA
caccttactttgttaa

SEQ ID NO: 851 - DNA, 17 bp, Brassica napus
  source 1..17: mol_type = genomic DNA
caccttactttgttaaa

SEQ ID NO: 852 - DNA, 18 bp, Brassica napus
  source 1..18: mol_type = genomic DNA
caccttactttgttaaat

SEQ ID NO: 853 - DNA, 19 bp, Brassica napus
  source 1..19: mol_type = genomic DNA
caccttactttgttaaatt

SEQ ID NO: 854 - DNA, 20 bp, Brassica napus
  source 1..20: mol_type = genomic DNA
caccttactttgttaaattt

SEQ ID NO: 855 - DNA, 21 bp, Brassica napus
  source 1..21: mol_type = genomic DNA
caccttactttgttaaattta

SEQ ID NO: 856 - DNA, 22 bp, Brassica napus
  source 1..22: mol_type = genomic DNA
caccttactttgttaaatttaa

SEQ ID NO: 857 - DNA, 23 bp, Brassica napus
  source 1..23: mol_type = genomic DNA
caccttactttgttaaatttaaa

SEQ ID NO: 858 - DNA, 15 bp, Brassica napus
  source 1..15: mol_type = genomic DNA
accttactttgttaa

SEQ ID NO: 859 - DNA, 16 bp, Brassica napus
  source 1..16: mol_type = genomic DNA
accttactttgttaaa

SEQ ID NO: 860 - DNA, 17 bp, Brassica napus
  source 1..17: mol_type = genomic DNA
accttactttgttaaat

SEQ ID NO: 861 - DNA, 18 bp, Brassica napus
  source 1..18: mol_type = genomic DNA
accttactttgttaaatt

SEQ ID NO: 862 - DNA, 19 bp, Brassica napus
  source 1..19: mol_type = genomic DNA
accttactttgttaaattt

SEQ ID NO: 863 - DNA, 20 bp, Brassica napus
  source 1..20: mol_type = genomic DNA
accttactttgttaaattta

SEQ ID NO: 864 - DNA, 21 bp, Brassica napus
  source 1..21: mol_type = genomic DNA
accttactttgttaaatttaa

SEQ ID NO: 865 - DNA, 22 bp, Brassica napus
  source 1..22: mol_type = genomic DNA
accttactttgttaaatttaaa

SEQ ID NO: 866 - DNA, 23 bp, Brassica napus
  source 1..23: mol_type = genomic DNA
accttactttgttaaatttaaaa

SEQ ID NO: 867 - DNA, 15 bp, Brassica napus
  source 1..15: mol_type = genomic DNA
ccttactttgttaaa

SEQ ID NO: 868 - DNA, 16 bp, Brassica napus
  source 1..16: mol_type = genomic DNA
ccttactttgttaaat

SEQ ID NO: 869 - DNA, 17 bp, Brassica napus
  source 1..17: mol_type = genomic DNA
ccttactttgttaaatt

SEQ ID NO: 870 - DNA, 18 bp, Brassica napus
  source 1..18: mol_type = genomic DNA
ccttactttgttaaattt

SEQ ID NO: 871 - DNA, 19 bp, Brassica napus
  source 1..19: mol_type = genomic DNA
ccttactttgttaaattta

SEQ ID NO: 872 - DNA, 20 bp, Brassica napus
  source 1..20: mol_type = genomic DNA
ccttactttgttaaatttaa

SEQ ID NO: 873 - DNA, 21 bp, Brassica napus
  source 1..21: mol_type = genomic DNA
ccttactttgttaaatttaaa

SEQ ID NO: 874 - DNA, 22 bp, Brassica napus
  source 1..22: mol_type = genomic DNA
ccttactttgttaaatttaaaa

SEQ ID NO: 875 - DNA, 23 bp, Brassica napus
  source 1..23: mol_type = genomic DNA
ccttactttgttaaatttaaaat

SEQ ID NO: 876 - DNA, 15 bp, Brassica napus
  source 1..15: mol_type = genomic DNA
cttactttgttaaat

SEQ ID NO: 877 - DNA, 16 bp, Brassica napus
  source 1..16: mol_type = genomic DNA
cttactttgttaaatt

SEQ ID NO: 878 - DNA, 17 bp, Brassica napus
  source 1..17: mol_type = genomic DNA
cttactttgttaaattt

SEQ ID NO: 879 - DNA, 18 bp, Brassica napus
  source 1..18: mol_type = genomic DNA
cttactttgttaaattta

SEQ ID NO: 880 - DNA, 19 bp, Brassica napus
  source 1..19: mol_type = genomic DNA
cttactttgttaaatttaa

SEQ ID NO: 881 - DNA, 20 bp, Brassica napus
  source 1..20: mol_type = genomic DNA
cttactttgttaaatttaaa

SEQ ID NO: 882 - DNA, 21 bp, Brassica napus
  source 1..21: mol_type = genomic DNA
cttactttgttaaatttaaaa

SEQ ID NO: 883 - DNA, 22 bp, Brassica napus
  source 1..22: mol_type = genomic DNA
cttactttgttaaatttaaaat

SEQ ID NO: 884 - DNA, 23 bp, Brassica napus
  source 1..23: mol_type = genomic DNA
cttactttgttaaatttaaaata

SEQ ID NO: 885 - DNA, 15 bp, Brassica napus
  source 1..15: mol_type = genomic DNA
ttactttgttaaatt

SEQ ID NO: 886 - DNA, 16 bp, Brassica napus
  source 1..16: mol_type = genomic DNA
ttactttgttaaattt

SEQ ID NO: 887 - DNA, 17 bp, Brassica napus
  source 1..17: mol_type = genomic DNA
ttactttgttaaattta

SEQ ID NO: 888 - DNA, 18 bp, Brassica napus
  source 1..18: mol_type = genomic DNA
ttactttgttaaatttaa

SEQ ID NO: 889 - DNA, 19 bp, Brassica napus
  source 1..19: mol_type = genomic DNA
ttactttgttaaatttaaa

SEQ ID NO: 890 - DNA, 20 bp, Brassica napus
  source 1..20: mol_type = genomic DNA
ttactttgttaaatttaaaa

SEQ ID NO: 891 - DNA, 21 bp, Brassica napus
  source 1..21: mol_type = genomic DNA
ttactttgttaaatttaaaat

SEQ ID NO: 892 - DNA, 22 bp, Brassica napus
  source 1..22: mol_type = genomic DNA
ttactttgttaaatttaaaata

SEQ ID NO: 893 - DNA, 23 bp, Brassica napus
  source 1..23: mol_type = genomic DNA
ttactttgttaaatttaaaatag

SEQ ID NO: 894 - DNA, 15 bp, Brassica napus
  source 1..15: mol_type = genomic DNA
ggccttcacgctccg

SEQ ID NO: 895 - DNA, 16 bp, Brassica napus
  source 1..16: mol_type = genomic DNA
ggccttcacgctccga

SEQ ID NO: 896 - DNA, 17 bp, Brassica napus
  source 1..17: mol_type = genomic DNA
ggccttcacgctccgat

SEQ ID NO: 897 - DNA, 18 bp, Brassica napus
  source 1..18: mol_type = genomic DNA
ggccttcacgctccgatg

SEQ ID NO: 898 - DNA, 19 bp, Brassica napus
  source 1..19: mol_type = genomic DNA
ggccttcacgctccgatgt

SEQ ID NO: 899 - DNA, 20 bp, Brassica napus
  source 1..20: mol_type = genomic DNA
ggccttcacgctccgatgtc

SEQ ID NO: 900 - DNA, 21 bp, Brassica napus
  source 1..21: mol_type = genomic DNA
ggccttcacgctccgatgtct

SEQ ID NO: 901 - DNA, 22 bp, Brassica napus
  source 1..22: mol_type = genomic DNA
ggccttcacgctccgatgtctt

SEQ ID NO: 902 - DNA, 23 bp, Brassica napus
  source 1..23: mol_type = genomic DNA
ggccttcacgctccgatgtcttc

SEQ ID NO: 903 - DNA, 15 bp, Brassica napus
  source 1..15: mol_type = genomic DNA
gccttcacgctccga

SEQ ID NO: 904 - DNA, 16 bp, Brassica napus
  source 1..16: mol_type = genomic DNA
gccttcacgctccgat

SEQ ID NO: 905 - DNA, 17 bp, Brassica napus
  source 1..17: mol_type = genomic DNA
gccttcacgctccgatg

SEQ ID NO: 906 - DNA, 18 bp, Brassica napus
  source 1..18: mol_type = genomic DNA
gccttcacgctccgatgt

SEQ ID NO: 907 - DNA, 19 bp, Brassica napus
  source 1..19: mol_type = genomic DNA
gccttcacgctccgatgtc

SEQ ID NO: 908 - DNA, 20 bp, Brassica napus
  source 1..20: mol_type = genomic DNA
gccttcacgctccgatgtct

SEQ ID NO: 909 - DNA, 21 bp, Brassica napus
  source 1..21: mol_type = genomic DNA
gccttcacgctccgatgtctt

SEQ ID NO: 910 - DNA, 22 bp, Brassica napus
  source 1..22: mol_type = genomic DNA
gccttcacgctccgatgtcttc

SEQ ID NO: 911 - DNA, 23 bp, Brassica napus
  source 1..23: mol_type = genomic DNA
gccttcacgctccgatgtcttct

SEQ ID NO: 912 - DNA, 15 bp, Brassica napus
  source 1..15: mol_type = genomic DNA
ccttcacgctccgat

SEQ ID NO: 913 - DNA, 16 bp, Brassica napus
  source 1..16: mol_type = genomic DNA
ccttcacgctccgatg

SEQ ID NO: 914 - DNA, 17 bp, Brassica napus
  source 1..17: mol_type = genomic DNA
ccttcacgctccgatgt

SEQ ID NO: 915 - DNA, 18 bp, Brassica napus
  source 1..18: mol_type = genomic DNA
ccttcacgctccgatgtc

SEQ ID NO: 916 - DNA, 19 bp, Brassica napus
  source 1..19: mol_type = genomic DNA
ccttcacgctccgatgtct

SEQ ID NO: 917 - DNA, 20 bp, Brassica napus
  source 1..20: mol_type = genomic DNA
ccttcacgctccgatgtctt

SEQ ID NO: 918 - DNA, 21 bp, Brassica napus
  source 1..21: mol_type = genomic DNA
ccttcacgctccgatgtcttc

SEQ ID NO: 919 - DNA, 22 bp, Brassica napus
  source 1..22: mol_type = genomic DNA
ccttcacgctccgatgtcttct

SEQ ID NO: 920 - DNA, 23 bp, Brassica napus
  source 1..23: mol_type = genomic DNA
ccttcacgctccgatgtcttctg

SEQ ID NO: 921 - DNA, 15 bp, Brassica napus
  source 1..15: mol_type = genomic DNA
cttcacgctccgatg

SEQ ID NO: 922 - DNA, 16 bp, Brassica napus
  source 1..16: mol_type = genomic DNA
cttcacgctccgatgt

SEQ ID NO: 923 - DNA, 17 bp, Brassica napus
  source 1..17: mol_type = genomic DNA
cttcacgctccgatgtc

SEQ ID NO: 924 - DNA, 18 bp, Brassica napus
  source 1..18: mol_type = genomic DNA
cttcacgctccgatgtct

SEQ ID NO: 925 - DNA, 19 bp, Brassica napus
  source 1..19: mol_type = genomic DNA
cttcacgctccgatgtctt

SEQ ID NO: 926 - DNA, 20 bp, Brassica napus
  source 1..20: mol_type = genomic DNA
cttcacgctccgatgtcttc

SEQ ID NO: 927 - DNA, 21 bp, Brassica napus
  source 1..21: mol_type = genomic DNA
cttcacgctccgatgtcttct

SEQ ID NO: 928 - DNA, 22 bp, Brassica napus
  source 1..22: mol_type = genomic DNA
cttcacgctccgatgtcttctg

SEQ ID NO: 929 - DNA, 23 bp, Brassica napus
  source 1..23: mol_type = genomic DNA
cttcacgctccgatgtcttctgt

SEQ ID NO: 930 - DNA, 15 bp, Brassica napus
  source 1..15: mol_type = genomic DNA
ttcacgctccgatgt

SEQ ID NO: 931 - DNA, 16 bp, Brassica napus
  source 1..16: mol_type = genomic DNA
ttcacgctccgatgtc

SEQ ID NO: 932 - DNA, 17 bp, Brassica napus
  source 1..17: mol_type = genomic DNA
ttcacgctccgatgtct

SEQ ID NO: 933 - DNA, 18 bp, Brassica napus
  source 1..18: mol_type = genomic DNA
ttcacgctccgatgtctt

SEQ ID NO: 934 - DNA, 19 bp, Brassica napus
  source 1..19: mol_type = genomic DNA
ttcacgctccgatgtcttc

SEQ ID NO: 935 - DNA, 20 bp, Brassica napus
  source 1..20: mol_type = genomic DNA
ttcacgctccgatgtcttct

SEQ ID NO: 936 - DNA, 21 bp, Brassica napus
  source 1..21: mol_type = genomic DNA
ttcacgctccgatgtcttctg

SEQ ID NO: 937 - DNA, 22 bp, Brassica napus
  source 1..22: mol_type = genomic DNA
ttcacgctccgatgtcttctgt

SEQ ID NO: 938 - DNA, 23 bp, Brassica napus
  source 1..23: mol_type = genomic DNA
ttcacgctccgatgtcttctgtg

SEQ ID NO: 939 - DNA, 15 bp, Brassica napus
  source 1..15: mol_type = genomic DNA
tcacgctccgatgtc

SEQ ID NO: 940 - DNA, 16 bp, Brassica napus
  source 1..16: mol_type = genomic DNA
tcacgctccgatgtct

SEQ ID NO: 941 - DNA, 17 bp, Brassica napus
  source 1..17: mol_type = genomic DNA
tcacgctccgatgtctt

SEQ ID NO: 942 - DNA, 18 bp, Brassica napus
  source 1..18: mol_type = genomic DNA
tcacgctccgatgtcttc

SEQ ID NO: 943 - DNA, 19 bp, Brassica napus
  source 1..19: mol_type = genomic DNA
tcacgctccgatgtcttct

SEQ ID NO: 944 - DNA, 20 bp, Brassica napus
  source 1..20: mol_type = genomic DNA
tcacgctccgatgtcttctg

SEQ ID NO: 945 - DNA, 21 bp, Brassica napus
  source 1..21: mol_type = genomic DNA
tcacgctccgatgtcttctgt

SEQ ID NO: 946 - DNA, 22 bp, Brassica napus
  source 1..22: mol_type = genomic DNA
tcacgctccgatgtcttctgtg

SEQ ID NO: 947 - DNA, 23 bp, Brassica napus
  source 1..23: mol_type = genomic DNA
tcacgctccgatgtcttctgtgt

SEQ ID NO: 948 - DNA, 15 bp, Brassica napus
  source 1..15: mol_type = genomic DNA
tttaactatagtggc

SEQ ID NO: 949 - DNA, 16 bp, Brassica napus
  source 1..16: mol_type = genomic DNA
tttaactatagtggcc

SEQ ID NO: 950 - DNA, 17 bp, Brassica napus
  source 1..17: mol_type = genomic DNA
tttaactatagtggcct

SEQ ID NO: 951 - DNA, 18 bp, Brassica napus
  source 1..18: mol_type = genomic DNA
tttaactatagtggcctt

SEQ ID NO: 952 - DNA, 19 bp, Brassica napus
  source 1..19: mol_type = genomic DNA
tttaactatagtggccttc

SEQ ID NO: 953 - DNA, 20 bp, Brassica napus
  source 1..20: mol_type = genomic DNA
tttaactatagtggccttca

SEQ ID NO: 954 - DNA, 21 bp, Brassica napus
  source 1..21: mol_type = genomic DNA
tttaactatagtggccttcac

SEQ ID NO: 955 - DNA, 22 bp, Brassica napus
  source 1..22: mol_type = genomic DNA
tttaactatagtggccttcacg

SEQ ID NO: 956 - DNA, 23 bp, Brassica napus
  source 1..23: mol_type = genomic DNA
tttaactatagtggccttcacgc

SEQ ID NO: 957 - DNA, 15 bp, Brassica napus
  source 1..15: mol_type = genomic DNA
cacgctccgatgtct

SEQ ID NO: 958 - DNA, 16 bp, Brassica napus
  source 1..16: mol_type = genomic DNA
cacgctccgatgtctt

SEQ ID NO: 959 - DNA, 17 bp, Brassica napus
  source 1..17: mol_type = genomic DNA
cacgctccgatgtcttc

SEQ ID NO: 960 - DNA, 18 bp, Brassica napus
  source 1..18: mol_type = genomic DNA
cacgctccgatgtcttct

SEQ ID NO: 961 - DNA, 19 bp, Brassica napus
  source 1..19: mol_type = genomic DNA
cacgctccgatgtcttctg

SEQ ID NO: 962 - DNA, 20 bp, Brassica napus
  source 1..20: mol_type = genomic DNA
cacgctccgatgtcttctgt

SEQ ID NO: 963 - DNA, 21 bp, Brassica napus
  source 1..21: mol_type = genomic DNA
cacgctccgatgtcttctgtg

SEQ ID NO: 964 - DNA, 22 bp, Brassica napus
  source 1..22: mol_type = genomic DNA
cacgctccgatgtcttctgtgt

SEQ ID NO: 965 - DNA, 23 bp, Brassica napus
  source 1..23: mol_type = genomic DNA
cacgctccgatgtcttctgtgtg

SEQ ID NO: 966 - DNA, 15 bp, Brassica napus
  source 1..15: mol_type = genomic DNA
acgctccgatgtctt

SEQ ID NO: 967 - DNA, 16 bp, Brassica napus
  source 1..16: mol_type = genomic DNA
acgctccgatgtcttc

SEQ ID NO: 968 - DNA, 17 bp, Brassica napus
  source 1..17: mol_type = genomic DNA
acgctccgatgtcttct

SEQ ID NO: 969 - DNA, 18 bp, Brassica napus
  source 1..18: mol_type = genomic DNA
acgctccgatgtcttctg

SEQ ID NO: 970 - DNA, 19 bp, Brassica napus
  source 1..19: mol_type = genomic DNA
acgctccgatgtcttctgt

SEQ ID NO: 971 - DNA, 20 bp, Brassica napus
  source 1..20: mol_type = genomic DNA
acgctccgatgtcttctgtg

SEQ ID NO: 972 - DNA, 21 bp, Brassica napus
  source 1..21: mol_type = genomic DNA
acgctccgatgtcttctgtgt

SEQ ID NO: 973 - DNA, 22 bp, Brassica napus
  source 1..22: mol_type = genomic DNA
acgctccgatgtcttctgtgtg

SEQ ID NO: 974 - DNA, 23 bp, Brassica napus
  source 1..23: mol_type = genomic DNA
acgctccgatgtcttctgtgtgg

SEQ ID NO: 975 - DNA, 15 bp, Brassica napus
  source 1..15: mol_type = genomic DNA
cgctccgatgtcttc

SEQ ID NO: 976 - DNA, 16 bp, Brassica napus
  source 1..16: mol_type = genomic DNA
cgctccgatgtcttct

SEQ ID NO: 977 - DNA, 17 bp, Brassica napus
  source 1..17: mol_type = genomic DNA
cgctccgatgtcttctg

SEQ ID NO: 978 - DNA, 18 bp, Brassica napus
  source 1..18: mol_type = genomic DNA
cgctccgatgtcttctgt

SEQ ID NO: 979 - DNA, 19 bp, Brassica napus
  source 1..19: mol_type = genomic DNA
cgctccgatgtcttctgtg

SEQ ID NO: 980 - DNA, 20 bp, Brassica napus
  source 1..20: mol_type = genomic DNA
cgctccgatgtcttctgtgt

SEQ ID NO: 981 - DNA, 21 bp, Brassica napus
  source 1..21: mol_type = genomic DNA
cgctccgatgtcttctgtgtg

SEQ ID NO: 982 - DNA, 22 bp, Brassica napus
  source 1..22: mol_type = genomic DNA
cgctccgatgtcttctgtgtgg

SEQ ID NO: 983 - DNA, 23 bp, Brassica napus
  source 1..23: mol_type = genomic DNA
cgctccgatgtcttctgtgtggg

SEQ ID NO: 984 - DNA, 15 bp, Brassica napus
  source 1..15: mol_type = genomic DNA
gctccgatgtcttct

SEQ ID NO: 985 - DNA, 16 bp, Brassica napus
  source 1..16: mol_type = genomic DNA
gctccgatgtcttctg

SEQ ID NO: 986 - DNA, 17 bp, Brassica napus
  source 1..17: mol_type = genomic DNA
gctccgatgtcttctgt

SEQ ID NO: 987 - DNA, 18 bp, Brassica napus
  source 1..18: mol_type = genomic DNA
gctccgatgtcttctgtg

SEQ ID NO: 988 - DNA, 19 bp, Brassica napus
  source 1..19: mol_type = genomic DNA
gctccgatgtcttctgtgt

SEQ ID NO: 989 - DNA, 20 bp, Brassica napus
  source 1..20: mol_type = genomic DNA
gctccgatgtcttctgtgtg

SEQ ID NO: 990 - DNA, 21 bp, Brassica napus
  source 1..21: mol_type = genomic DNA
gctccgatgtcttctgtgtgg

SEQ ID NO: 991 - DNA, 22 bp, Brassica napus
  source 1..22: mol_type = genomic DNA
gctccgatgtcttctgtgtggg

SEQ ID NO: 992 - DNA, 23 bp, Brassica napus
  source 1..23: mol_type = genomic DNA
gctccgatgtcttctgtgtgggc

SEQ ID NO: 993 - DNA, 15 bp, Brassica napus
  source 1..15: mol_type = genomic DNA
ctccgatgtcttctg

SEQ ID NO: 994 - DNA, 16 bp, Brassica napus
  source 1..16: mol_type = genomic DNA
ctccgatgtcttctgt

SEQ ID NO: 995 - DNA, 17 bp, Brassica napus
  source 1..17: mol_type = genomic DNA
ctccgatgtcttctgtg

SEQ ID NO: 996 - DNA, 18 bp, Brassica napus
  source 1..18: mol_type = genomic DNA
ctccgatgtcttctgtgt

SEQ ID NO: 997 - DNA, 19 bp, Brassica napus
  source 1..19: mol_type = genomic DNA
ctccgatgtcttctgtgtg

SEQ ID NO: 998 - DNA, 20 bp, Brassica napus
  source 1..20: mol_type = genomic DNA
ctccgatgtcttctgtgtgg

SEQ ID NO: 999 - DNA, 21 bp, Brassica napus
  source 1..21: mol_type = genomic DNA
ctccgatgtcttctgtgtggg

SEQ ID NO: 1000 - DNA, 22 bp, Brassica napus
  source 1..22: mol_type = genomic DNA
ctccgatgtcttctgtgtgggc

SEQ ID NO: 1001 - DNA, 23 bp, Brassica napus
  source 1..23: mol_type = genomic DNA
ctccgatgtcttctgtgtgggct

SEQ ID NO: 1002 - DNA, 15 bp, Brassica napus
  source 1..15: mol_type = genomic DNA
tccgatgtcttctgt

SEQ ID NO: 1003 - DNA, 16 bp, Brassica napus
  source 1..16: mol_type = genomic DNA
tccgatgtcttctgtg

SEQ ID NO: 1004 - DNA, 17 bp, Brassica napus
  source 1..17: mol_type = genomic DNA
tccgatgtcttctgtgt

SEQ ID NO: 1005 - DNA, 18 bp, Brassica napus
  source 1..18: mol_type = genomic DNA
tccgatgtcttctgtgtg

SEQ ID NO: 1006 - DNA, 19 bp, Brassica napus
  source 1..19: mol_type = genomic DNA
tccgatgtcttctgtgtgg

SEQ ID NO: 1007 - DNA, 20 bp, Brassica napus
  source 1..20: mol_type = genomic DNA
tccgatgtcttctgtgtggg

SEQ ID NO: 1008 - DNA, 21 bp, Brassica napus
  source 1..21: mol_type = genomic DNA
tccgatgtcttctgtgtgggc

SEQ ID NO: 1009 - DNA, 22 bp, Brassica napus
  source 1..22: mol_type = genomic DNA
tccgatgtcttctgtgtgggct

SEQ ID NO: 1010 - DNA, 23 bp, Brassica napus
  source 1..23: mol_type = genomic DNA
tccgatgtcttctgtgtgggctt

SEQ ID NO: 1011 - DNA, 15 bp, Brassica napus
  source 1..15: mol_type = genomic DNA
ccgatgtcttctgtg

SEQ ID NO: 1012 - DNA, 16 bp, Brassica napus
  source 1..16: mol_type = genomic DNA
ccgatgtcttctgtgt

SEQ ID NO: 1013 - DNA, 17 bp, Brassica napus
  source 1..17: mol_type = genomic DNA
ccgatgtcttctgtgtg

SEQ ID NO: 1014 - DNA, 18 bp, Brassica napus
  source 1..18: mol_type = genomic DNA
ccgatgtcttctgtgtgg

SEQ ID NO: 1015 - DNA, 19 bp, Brassica napus
  source 1..19: mol_type = genomic DNA
ccgatgtcttctgtgtggg

SEQ ID NO: 1016 - DNA, 20 bp, Brassica napus
  source 1..20: mol_type = genomic DNA
ccgatgtcttctgtgtgggc

SEQ ID NO: 1017 - DNA, 21 bp, Brassica napus
  source 1..21: mol_type = genomic DNA
ccgatgtcttctgtgtgggct

SEQ ID NO: 1018 - DNA, 22 bp, Brassica napus
  source 1..22: mol_type = genomic DNA
ccgatgtcttctgtgtgggctt

SEQ ID NO: 1019 - DNA, 23 bp, Brassica napus
  source 1..23: mol_type = genomic DNA
ccgatgtcttctgtgtgggcttt

SEQ ID NO: 1020 - DNA, 15 bp, Brassica napus
  source 1..15: mol_type = genomic DNA
cgatgtcttctgtgt

SEQ ID NO: 1021 - DNA, 16 bp, Brassica napus
  source 1..16: mol_type = genomic DNA
cgatgtcttctgtgtg

SEQ ID NO: 1022 - DNA, 17 bp, Brassica napus
  source 1..17: mol_type = genomic DNA
cgatgtcttctgtgtgg

SEQ ID NO: 1023 - DNA, 18 bp, Brassica napus
  source 1..18: mol_type = genomic DNA
cgatgtcttctgtgtggg

SEQ ID NO: 1024 - DNA, 19 bp, Brassica napus
  source 1..19: mol_type = genomic DNA
cgatgtcttctgtgtgggc

SEQ ID NO: 1025 - DNA, 20 bp, Brassica napus
  source 1..20: mol_type = genomic DNA
cgatgtcttctgtgtgggct

SEQ ID NO: 1026 - DNA, 21 bp, Brassica napus
  source 1..21: mol_type = genomic DNA
cgatgtcttctgtgtgggctt

SEQ ID NO: 1027 - DNA, 22 bp, Brassica napus
  source 1..22: mol_type = genomic DNA
cgatgtcttctgtgtgggcttt

SEQ ID NO: 1028 - DNA, 23 bp, Brassica napus
  source 1..23: mol_type = genomic DNA
cgatgtcttctgtgtgggcttta

SEQ ID NO: 1029 - DNA, 15 bp, Brassica napus
  source 1..15: mol_type = genomic DNA
gatgtcttctgtgtg

SEQ ID NO: 1030 - DNA, 16 bp, Brassica napus
  source 1..16: mol_type = genomic DNA
gatgtcttctgtgtgg

SEQ ID NO: 1031 - DNA, 17 bp, Brassica napus
  source 1..17: mol_type = genomic DNA
gatgtcttctgtgtggg

SEQ ID NO: 1032 - DNA, 18 bp, Brassica napus
  source 1..18: mol_type = genomic DNA
gatgtcttctgtgtgggc

SEQ ID NO: 1033 - DNA, 19 bp, Brassica napus
  source 1..19: mol_type = genomic DNA
gatgtcttctgtgtgggct

SEQ ID NO: 1034 - DNA, 20 bp, Brassica napus
  source 1..20: mol_type = genomic DNA
gatgtcttctgtgtgggctt

SEQ ID NO: 1035 - DNA, 21 bp, Brassica napus
  source 1..21: mol_type = genomic DNA
gatgtcttctgtgtgggcttt

SEQ ID NO: 1036 - DNA, 22 bp, Brassica napus
  source 1..22: mol_type = genomic DNA
gatgtcttctgtgtgggcttta

SEQ ID NO: 1037 - DNA, 23 bp, Brassica napus
  source 1..23: mol_type = genomic DNA
gatgtcttctgtgtgggctttat

SEQ ID NO: 1038 - DNA, 15 bp, Brassica napus
  source 1..15: mol_type = genomic DNA
atgtcttctgtgtgg

SEQ ID NO: 1039 - DNA, 16 bp, Brassica napus
  source 1..16: mol_type = genomic DNA
atgtcttctgtgtggg

SEQ ID NO: 1040 - DNA, 17 bp, Brassica napus
  source 1..17: mol_type = genomic DNA
atgtcttctgtgtgggc

SEQ ID NO: 1041 - DNA, 18 bp, Brassica napus
  source 1..18: mol_type = genomic DNA
atgtcttctgtgtgggct

SEQ ID NO: 1042 - DNA, 19 bp, Brassica napus
  source 1..19: mol_type = genomic DNA
atgtcttctgtgtgggctt

SEQ ID NO: 1043 - DNA, 20 bp, Brassica napus
  source 1..20: mol_type = genomic DNA
atgtcttctgtgtgggcttt

SEQ ID NO: 1044 - DNA, 21 bp, Brassica napus
  source 1..21: mol_type = genomic DNA
atgtcttctgtgtgggcttta

SEQ ID NO: 1045 - DNA, 22 bp, Brassica napus
  source 1..22: mol_type = genomic DNA
atgtcttctgtgtgggctttat

SEQ ID NO: 1046 - DNA, 23 bp, Brassica napus
  source 1..23: mol_type = genomic DNA
atgtcttctgtgtgggctttatg

SEQ ID NO: 1047 - DNA, 15 bp, Brassica napus
  source 1..15: mol_type = genomic DNA
ttaactatagtggcc

SEQ ID NO: 1048 - DNA, 16 bp, Brassica napus
  source 1..16: mol_type = genomic DNA
ttaactatagtggcct

SEQ ID NO: 1049 - DNA, 17 bp, Brassica napus
  source 1..17: mol_type = genomic DNA
ttaactatagtggcctt

SEQ ID NO: 1050 - DNA, 18 bp, Brassica napus
  source 1..18: mol_type = genomic DNA
ttaactatagtggccttc

SEQ ID NO: 1051 - DNA, 19 bp, Brassica napus
  source 1..19: mol_type = genomic DNA
ttaactatagtggccttca

SEQ ID NO: 1052 - DNA, 20 bp, Brassica napus
  source 1..20: mol_type = genomic DNA
ttaactatagtggccttcac

SEQ ID NO: 1053 - DNA, 21 bp, Brassica napus
  source 1..21: mol_type = genomic DNA
ttaactatagtggccttcacg

SEQ ID NO: 1054 - DNA, 22 bp, Brassica napus
  source 1..22: mol_type = genomic DNA
ttaactatagtggccttcacgc

SEQ ID NO: 1055 - DNA, 23 bp, Brassica napus
  source 1..23: mol_type = genomic DNA
ttaactatagtggccttcacgct

SEQ ID NO: 1056 - DNA, 15 bp, Brassica napus
  source 1..15: mol_type = genomic DNA
tgtcttctgtgtggg

SEQ ID NO: 1057 - DNA, 16 bp, Brassica napus
  source 1..16: mol_type = genomic DNA
tgtcttctgtgtgggc

SEQ ID NO: 1058 - DNA, 17 bp, Brassica napus
  source 1..17: mol_type = genomic DNA
tgtcttctgtgtgggct

SEQ ID NO: 1059 - DNA, 18 bp, Brassica napus
  source 1..18: mol_type = genomic DNA
tgtcttctgtgtgggctt

SEQ ID NO: 1060 - DNA, 19 bp, Brassica napus
  source 1..19: mol_type = genomic DNA
tgtcttctgtgtgggcttt

SEQ ID NO: 1061 - DNA, 20 bp, Brassica napus
  source 1..20: mol_type = genomic DNA
tgtcttctgtgtgggcttta

SEQ ID NO: 1062 - DNA, 21 bp, Brassica napus
  source 1..21: mol_type = genomic DNA
tgtcttctgtgtgggctttat

SEQ ID NO: 1063 - DNA, 22 bp, Brassica napus
  source 1..22: mol_type = genomic DNA
tgtcttctgtgtgggctttatg

SEQ ID NO: 1064 - DNA, 23 bp, Brassica napus
  source 1..23: mol_type = genomic DNA
tgtcttctgtgtgggctttatga

SEQ ID NO: 1065 - DNA, 15 bp, Brassica napus
  source 1..15: mol_type = genomic DNA
gtcttctgtgtgggc

SEQ ID NO: 1066 - DNA, 16 bp, Brassica napus
  source 1..16: mol_type = genomic DNA
gtcttctgtgtgggct

SEQ ID NO: 1067 - DNA, 17 bp, Brassica napus
  source 1..17: mol_type = genomic DNA
gtcttctgtgtgggctt

SEQ ID NO: 1068 - DNA, 18 bp, Brassica napus
  source 1..18: mol_type = genomic DNA
gtcttctgtgtgggcttt

SEQ ID NO: 1069 - DNA, 19 bp, Brassica napus
  source 1..19: mol_type = genomic DNA
gtcttctgtgtgggcttta

SEQ ID NO: 1070 - DNA, 20 bp, Brassica napus
  source 1..20: mol_type = genomic DNA
gtcttctgtgtgggctttat

SEQ ID NO: 1071 - DNA, 21 bp, Brassica napus
  source 1..21: mol_type = genomic DNA
gtcttctgtgtgggctttatg

SEQ ID NO: 1072 - DNA, 22 bp, Brassica napus
  source 1..22: mol_type = genomic DNA
gtcttctgtgtgggctttatga

SEQ ID NO: 1073 - DNA, 23 bp, Brassica napus
  source 1..23: mol_type = genomic DNA
gtcttctgtgtgggctttatgat

SEQ ID NO: 1074 - DNA, 15 bp, Brassica napus
  source 1..15: mol_type = genomic DNA
tcttctgtgtgggct

SEQ ID NO: 1075 - DNA, 16 bp, Brassica napus
  source 1..16: mol_type = genomic DNA
tcttctgtgtgggctt

SEQ ID NO: 1076 - DNA, 17 bp, Brassica napus
  source 1..17: mol_type = genomic DNA
tcttctgtgtgggcttt

SEQ ID NO: 1077 - DNA, 18 bp, Brassica napus
  source 1..18: mol_type = genomic DNA
tcttctgtgtgggcttta

SEQ ID NO: 1078 - DNA, 19 bp, Brassica napus
  source 1..19: mol_type = genomic DNA
tcttctgtgtgggctttat

SEQ ID NO: 1079 - DNA, 20 bp, Brassica napus
  source 1..20: mol_type = genomic DNA
tcttctgtgtgggctttatg

SEQ ID NO: 1080 - DNA, 21 bp, Brassica napus
  source 1..21: mol_type = genomic DNA
tcttctgtgtgggctttatga

SEQ ID NO: 1081 - DNA, 22 bp, Brassica napus
  source 1..22: mol_type = genomic DNA
tcttctgtgtgggctttatgat

SEQ ID NO: 1082 - DNA, 23 bp, Brassica napus
  source 1..23: mol_type = genomic DNA
tcttctgtgtgggctttatgata

SEQ ID NO: 1083 - DNA, 15 bp, Brassica napus
  source 1..15: mol_type = genomic DNA
cttctgtgtgggctt

SEQ ID NO: 1084 - DNA, 16 bp, Brassica napus
  source 1..16: mol_type = genomic DNA
cttctgtgtgggcttt

SEQ ID NO: 1085 - DNA, 17 bp, Brassica napus
  source 1..17: mol_type = genomic DNA
cttctgtgtgggcttta

SEQ ID NO: 1086 - DNA, 18 bp, Brassica napus
  source 1..18: mol_type = genomic DNA
cttctgtgtgggctttat

SEQ ID NO: 1087 - DNA, 19 bp, Brassica napus
  source 1..19: mol_type = genomic DNA
cttctgtgtgggctttatg

SEQ ID NO: 1088 - DNA, 20 bp, Brassica napus
  source 1..20: mol_type = genomic DNA
cttctgtgtgggctttatga

SEQ ID NO: 1089 - DNA, 21 bp, Brassica napus
  source 1..21: mol_type = genomic DNA
cttctgtgtgggctttatgat

SEQ ID NO: 1090 - DNA, 22 bp, Brassica napus
  source 1..22: mol_type = genomic DNA
cttctgtgtgggctttatgata

SEQ ID NO: 1091 - DNA, 23 bp, Brassica napus
  source 1..23: mol_type = genomic DNA
cttctgtgtgggctttatgataa

SEQ ID NO: 1092 - DNA, 15 bp, Brassica napus
  source 1..15: mol_type = genomic DNA
ttctgtgtgggcttt

SEQ ID NO: 1093 - DNA, 16 bp, Brassica napus
  source 1..16: mol_type = genomic DNA
ttctgtgtgggcttta

SEQ ID NO: 1094 - DNA, 17 bp, Brassica napus
  source 1..17: mol_type = genomic DNA
ttctgtgtgggctttat

SEQ ID NO: 1095 - DNA, 18 bp, Brassica napus
  source 1..18: mol_type = genomic DNA
ttctgtgtgggctttatg

SEQ ID NO: 1096 - DNA, 19 bp, Brassica napus
  source 1..19: mol_type = genomic DNA
ttctgtgtgggctttatga

SEQ ID NO: 1097 - DNA, 20 bp, Brassica napus
  source 1..20: mol_type = genomic DNA
ttctgtgtgggctttatgat

SEQ ID NO: 1098 - DNA, 21 bp, Brassica napus
  source 1..21: mol_type = genomic DNA
ttctgtgtgggctttatgata

SEQ ID NO: 1099 - DNA, 22 bp, Brassica napus
  source 1..22: mol_type = genomic DNA
ttctgtgtgggctttatgataa

SEQ ID NO: 1100 - DNA, 23 bp, Brassica napus
  source 1..23: mol_type = genomic DNA
ttctgtgtgggctttatgataaa

SEQ ID NO: 1101 - DNA, 15 bp, Brassica napus
  source 1..15: mol_type = genomic DNA
tctgtgtgggcttta

SEQ ID NO: 1102 - DNA, 16 bp, Brassica napus
  source 1..16: mol_type = genomic DNA
tctgtgtgggctttat

SEQ ID NO: 1103 - DNA, 17 bp, Brassica napus
  source 1..17: mol_type = genomic DNA
tctgtgtgggctttatg

SEQ ID NO: 1104 - DNA, 18 bp, Brassica napus
  source 1..18: mol_type = genomic DNA
tctgtgtgggctttatga

SEQ ID NO: 1105 - DNA, 19 bp, Brassica napus
  source 1..19: mol_type = genomic DNA
tctgtgtgggctttatgat

SEQ ID NO: 1106 - DNA, 20 bp, Brassica napus
  source 1..20: mol_type = genomic DNA
tctgtgtgggctttatgata

SEQ ID NO: 1107 - DNA, 21 bp, Brassica napus
  source 1..21: mol_type = genomic DNA
tctgtgtgggctttatgataa

SEQ ID NO: 1108 - DNA, 22 bp, Brassica napus
  source 1..22: mol_type = genomic DNA
tctgtgtgggctttatgataaa

SEQ ID NO: 1109 - DNA, 23 bp, Brassica napus
  source 1..23: mol_type = genomic DNA
tctgtgtgggctttatgataaaa

SEQ ID NO: 1110 - DNA, 15 bp, Brassica napus
  source 1..15: mol_type = genomic DNA
ctgtgtgggctttat

SEQ ID NO: 1111 - DNA, 16 bp, Brassica napus
  source 1..16: mol_type = genomic DNA
ctgtgtgggctttatg

SEQ ID NO: 1112 - DNA, 17 bp, Brassica napus
  source 1..17: mol_type = genomic DNA
ctgtgtgggctttatga

SEQ ID NO: 1113 - DNA, 18 bp, Brassica napus
  source 1..18: mol_type = genomic DNA
ctgtgtgggctttatgat

SEQ ID NO: 1114 - DNA, 19 bp, Brassica napus
  source 1..19: mol_type = genomic DNA
ctgtgtgggctttatgata

SEQ ID NO: 1115 - DNA, 20 bp, Brassica napus
  source 1..20: mol_type = genomic DNA
ctgtgtgggctttatgataa